(19) United States
(12) Patent Application Publication
(10) Pub. No.: US 2012/0045393 A1
(43) Pub. Date: Feb. 23, 2012

(54) LHRH-II PEPTIDE ANALOGS

(76) Inventors: Karen E. Linder, Kingston, NJ (US); Palaniappa Nanjappan, Princeton, NJ (US); Natarajan Raju, Kendall Park, NJ (US); Sudha Khurana, San Jose, CA (US); Rolf E. Swenson, Princeton, NJ (US); Adrian D. Nunn, Lambertville, NJ (US); Kondareddiar Ramalingam, Dayton, NJ (US)

(21) Appl. No.: 13/256,912

(22) PCT Filed: Mar. 16, 2010
(86) PCT No.: PCT/US10/27533

§ 371 Date: Sep. 15, 2011

Related U.S. Application Data
(60) Provisional application No. 61/160,887, filed on Mar. 17, 2009.

(51) Int. Cl. A61K 49/00; A61K 38/08; A61K 49/14; A61K 38/09; A61K 49/22; A61K 51/08; A61P 35/00; C07K 7/23; C07K 7/06; A61K 49/04

(52) U.S. Cl. 424/1.69; 530/328; 514/21.6; 424/9.1; 424/9.34; 424/9.4; 424/9.5; 514/19.5; 514/19.3; 514/19.4; 530/313; 514/9.7

(57) ABSTRACT

The invention is directed to analogs of LHRH-II and, more generally, to analogs of the LHRH family in which modifications have been made that confer enhanced binding affinity for LHRH receptors and/or improved metabolic stability.

The invention is further directed to methods of targeted therapy and targeted imaging in patients with sex-hormone-related cancers or other LHRH-mediated diseases.

This application is a 371 of International Application No. PCT/US2010/027533, filed Mar. 16, 2010.

The text file of the Sequence Listing submitted concurrently herewith, having the file name LHRH_ST25.txt, created on Jun. 11, 2010 and having a size of 106,104 bytes, is incorporated herein in its entirety by reference.

### BACKGROUND OF THE INVENTION

Gonadotropin releasing hormone (GnRH), also known as gonadotropin releasing factor (GnRF) or luteinizing hormone-releasing hormone (LHRH-I), is a decapeptide (pGlu-His-Trp-Ser-Tyr-Gly-Leu-Arg-Pro-Gly-NH₂) (SEQ ID NO: 1) that is secreted from the hypothalamus in a pulsatile pattern and acts upon its receptor in the anterior pituitary gland, thus regulating the production and release of the gonadotropins.^{1,2} LHRH-I was also found to be expressed in extra-hypothalamic regions of the central nervous system³ as well as in non-neuronal tissues such as placenta,⁴ ovary,⁵ mammary gland⁶ and lymphoid cells.⁷ In addition, LHRH-I and its receptor were found to be expressed in a number of malignant tumors and cell lines, including cancers of the breast, ovary, endometrium and prostate.

The gonadotropins Luteinizing Hormone (LH) and Follicle-Stimulating Hormone (FSH) stimulate sex steroid hormone synthesis and gametogenesis in the gonads to ensure normal reproductive function. LHRH antagonists cause rapid and reversible suppression of gonadotropin secretion by competing with endogenous LHRH-I for receptor binding.⁸ Continuous stimulation of pituitary LHRH receptor by exogenously administered LHRH-I agonists results in receptor desensitization and downregulation leading to an inhibition of pituitary gonadotropin secretion and a decline in ovarian and testicular function.⁹⁻¹¹ LHRH-I and its synthetic analogs including Leuprolide™ ([DLeu-6, desGly-10]LHRH-NH-Et) are used extensively for the treatment of hormone-dependent diseases such as endometriosis, uterine fibroids, benign prostate hyperplasia, fertility disorders, and precocious puberty, as well as prostate, ovarian and breast cancer and are also used in assisted reproductive techniques.¹²⁻¹⁴ In the therapy of prostate cancer, chronic administration of LHRH agonists such as Leuprolide™ Decapeptyl™ and Buserelin™, and antagonists Cetrorelix™ and Ganirelix™ results in medical castration.⁸

In the past few years the biology of LHRH has been revised due to accumulating evidence that extrapituitary, normal and malignant tissues locally produce the hormone and express LHRH binding sites,¹⁵ suggesting that LHRH agonists and antagonists may also have actions at these peripheral targets. Though it was initially thought that LHRH-I was unique, seven isoforms of LHRH have been identified in the brains of non-mammalian vertebrates. They are all decapeptides in which residues 1, 2, 4, 9, and 10 are conserved; position 8 is most variable.¹⁶ (Table 1).

TABLE 1
Primary Structures of Various LHRH Analogs Isolated from Vertebrate Brain

| No | LHRH Type | AA¹ | AA² | AA³ | AA⁴ | AA⁵ | AA⁶ | AA⁷ | AA⁸ | AA⁹ | AA¹⁰ | SEQ ID NO: |
|---|---|---|---|---|---|---|---|---|---|---|---|---|
| 1 | Mammal | pGlu | His | Trp | Ser | Tyr | Gly | Leu | Arg | Pro | Gly-NH₂ | 1 |
| | (LHRH-I)¹⁷ | | | | | | | | | | | |
| 2 | Chicken II | pGlu | His | Trp | Ser | His | Gly | Trp | Tyr | Pro | Gly-NH₂ | 2 |
| | (LHRH-II)¹⁸ | | | | | | | | | | | |
| 3 | Chicken I¹⁹ | pGlu | His | Trp | Ser | Tyr | Gly | Leu | Gln | Pro | Gly-NH₂ | 3 |
| 4 | Catfish²⁰ | pGlu | His | Trp | Ser | His | Gly | Leu | Asn | Pro | Gly-NH₂ | 4 |
| 5 | Salmon²¹ | pGlu | His | Trp | Ser | Tyr | Gly | Trp | Leu | Pro | Gly-NH₂ | 5 |
| 6 | Dogfish²² | pGlu | His | Trp | Ser | His | Gly | Trp | Leu | Pro | Gly-NH₂ | 6 |
| 7 | Lamprey²³ | pGlu | His | Tyr | Ser | Leu | Glu | Trp | Lys | Pro | Gly-NH₂ | 7 |

All forms have a blocked NH₂ and COOH terminus and invariant amino acids in positions 1, 2, 4, 9 and 10

LHRH-II was originally identified from chicken hypothalamus, but has also been found in humans.²⁴⁻²⁸ The LHRH-II isoform differs from LHRH-I at positions 5, 7 and 8 (His⁵, Trp⁷, Tyr⁸-LHRH-I); the structure of this isoform is completely conserved in fish to mammals.²⁹ In humans, extra-pituitary LHRH-II actions, such as suppression of tumor proliferation³⁰⁻³² have been demonstrated, even though a full-length LHRH-II receptor transcript has not yet been identified in any human tissues or cell types. The expression of mRNA for LHRH-II from human granulose cells in vitro³³ and from human endometrium³⁴ has been reported. Recently, Miller et al cloned a type II LHRH receptor from the marmoset monkey which was shown to be highly selective for LHRH-II.³⁵ Simultaneously, Neil et al³⁶ cloned the LHRH-II receptor from the rhesus monkey. Gründker et al³⁷ convincingly showed the expression of LHRH-II receptor mRNA in human endometrial and ovarian cancer cell lines using RT-PCR and Southern blot analysis. These authors also proved that a time- and dose-dependent administration of native LHRH-II significantly reduced the proliferation of human endometrial and ovarian cancer cell lines. The potent activity of LHRH-II and its analogs on the inhibition of progesterone production in ovary and hCG release in placenta led to the belief that LHRH-II might regulate reproductive tissue functions related to ovulation and fertilization.³⁸

Siler-Khodr (U.S. Pat. No. 6,323,179) disclosed analogs of LHRH-II and salmon LHRH that were designed to have enhanced and preferential binding to human chorionic LHRH receptor and ovarian LHRH receptors, and also to be resistant to degradation by chorionic peptidase 1. The analog peptides contained substitutions for the amino acid residues normally found at positions 6 and 10 of the native decapeptides.

Normal and malignant human breast tissues as well as breast cell lines (including MCF-7) secrete both LHRH-I and LHRH-II and express LHRH binding sites.³⁹ Several LHRH-1 agonists have been approved for the treatment of prostate cancer as well as other hormonally driven diseases such as endometriosis and uterine fibroids. The LHRH-1 antagonists Cetrorelix, Abarelix and Ganirelix have been approved for in vitro fertilization and Abarelix has been approved for treating prostate cancer. However, hormone deprivation does not prevent relapse and there is a need for more effective therapies.

The transportation of cytotoxic drugs such as Doxorubicin to peripheral LHRH receptors that are overexpressed on cancer cells has been accomplished with both LHRH antagonists and agonists, for example, by coupling cytotoxic drugs to the Lys at position 6 of the high affinity LHRH-I compound [D-Lys-6]LHRH.⁴⁰⁻⁴² Such compounds are reported to retain their activity both in vitro and in vivo.⁴¹ Cytotoxic metal complexes containing platinum, nickel and copper attached to the side chain of lysine at position 6 have demonstrated high in vitro activity in human breast tumor cells.

The effects noted by this group indicated that the native LHRH-II is statistically more potent than the antiproliferative effects of equimolar doses of the LHRH-I agonist triptorelin. In another study using LHRH-II-receptor-positive but LHRH-1-receptor-negative ovarian SK-OV-3 cell lines, native LHRH-II peptide showed antiproliferative effect, whereas LHRH-I did not.³⁷ These findings and other results described above have opened a new field of research on the role of LHRH-II in human cancers. LHRH-II receptor-targeted peptide-analog agonists/antagonists, both in unconjugated form and conjugated to chelators, may offer a new avenue of therapy for these cancers.

### SUMMARY OF THE INVENTION

The present invention is directed to new peptides and conjugates of those peptides useful in targeted therapy and targeted imaging in patients with diseases of the reproductive system, particularly patients with prostate, ovarian or breast cancer. More particularly, the peptides are primarily analogs of the decapeptide LHRH-II which have higher target-binding affinity and/or improved metabolic stability over the native form. The analogs may be in unconjugated form or they may be conjugated at the N-terminus and/or the C-terminus to a component containing a detectable label.

The principal such component is a chelator, preferably complexed with a metal radionuclide. Analog peptides containing such a component are useful both in targeted radiotherapy and in targeted scintigraphic imaging, such as SPECT or PET imaging.

The conjugated component may instead contain a label detectable by any one of a number of alternative known imaging techniques, for example, ultrasound or optical imaging. The resultant peptides are useful in targeted imaging in a patient.

Unconjugated peptide analogs according to the present invention are also useful in the targeted therapy of cancer patients.

The invention is further concerned with methods of treatment and imaging of cancer employing the peptide analogs and conjugates thereof.

### BRIEF DESCRIPTION OF THE DRAWINGS

FIG. 1 depicts radioactivity traces for the plasma samples obtained at 2 and 10 min post injection of ¹⁷⁷Lu-BRU-2813 in normal mice. The retention time of ¹⁷⁷Lu-BRU-2813 is ˜42 min in this system.

FIG. 2 depicts radioactivity traces for urine samples obtained at 10, 30 and 60 min post injection of ¹⁷⁷Lu-BRU-2813. ¹⁷⁷Lu-BRU-2813 formulation solution is shown as a control on the bottom panel.

FIG. 3 depicts radiochromatograms for ¹⁷⁷Lu-BRU-2813 incubated in kidney homogenate at 37° C. for 10 and 60 min, with ¹⁷⁷Lu-BRU-2813 formulation solution as a control (bottom panel). Extensive metabolism was seen.

FIG. 4 depicts radiochromatograms for ¹⁷⁷Lu-BRU-2813 incubated in liver homogenate at 37° C. for 10 and 60 min, with ¹⁷⁷Lu-BRU-2813 formulation solution as a control (bottom panel). Extensive metabolism was seen.

FIG. 5 depicts LC/MS analysis (ion current) of metabolites obtained when Lu-BRU-2813 was incubated in kidney homogenate at 37° C. for 1 h. Unmetabolized Lu-BRU-2813 has a retention time of 16.6 minutes in this system. The two major metabolites have retention times of 11.4 and 18.3 min.

FIG. 6 depicts a comparison of the chromatographic elution patterns of several Lu-derivatives of peptide BRU-2813, following incubation in liver homogenate, with that of a known Lu-BRU-2813 metabolite, Lu-BRU-3064.

FIG. 7 depicts a comparison of the chromatographic elution pattern of an additional derivative (Lu-BRU-2996) of peptide BRU-2813, following incubation in liver homogenate.

FIG. 8 depicts a comparison of the UV and ion-current traces of the chromatographic elution patterns of Lu-BRU-2996 following incubation in liver homogenate.

FIG. 9 shows the results of API-ES positive-mode analysis of the unmetabolized Lu-BRU-2996 remaining after incubation in liver homogenate.

FIG. 10 shows the results of API-ES positive-mode analysis of a metabolite of Lu-BRU-2996 following incubation in liver homogenate.

FIG. 11 depicts the UV trace of the chromatographic elution pattern of peptide BRU-2477 following incubation in liver homogenate.

FIG. 12 depicts the results of API-ES analysis of the peak eluting at 13.9 minutes in FIG. 11.

FIG. 13 depicts the results of API-ES analysis of the peak eluting at 14.6 minutes in FIG. 11.

FIG. 14 provides a comparison of the UV-traced chromatographic elution patterns of peptide BRU-3122 pre- and post-incubation in liver homogenate. Very little metabolism was observed.

FIG. 15 provides a comparison of the UV-traced chromatographic elution patterns of peptide BRU-3123 pre- and post-incubation in liver homogenate. Very little metabolism was observed.

FIG. 16 provides a comparison of the UV-traced chromatographic elution patterns of peptide BRU-3124 pre- and post-incubation in liver homogenate.

FIG. 17 depicts a comparison of the UV-traced chromatographic elution pattern of nonincubated peptide BRU-2477 with the patterns of peptides BRU-2477 and -3124 following incubation in liver homogenate.

FIG. 18 depicts a comparison of total and nonspecific binding of various ¹⁷⁷Lu-LHRH-II analogs to EFO-27 cancer cells.

FIGS. 19a and 19b are graphic depictions of the correlation between IC₅₀ values and % direct binding of ¹⁷⁷Lu-labeled LHRH complexes determined from studies in which several LHRH-II analogs were incubated with EFO-27 cells.

FIGS. 20a and b are graphs comparing the saturation binding of ¹²⁵I-LHRH-II and ¹⁷⁷Lu-BRU-2666 to EFO-27 cells.

FIGS. 21a-h are graphic depictions of the results of comparative time-course studies of internalization and efflux of radioactively labeled ¹²⁵I-LHRH-II and various radioactively labeled ¹⁷⁷Lu-LHRH-II analogs in EFO-27 cancer cells.

FIGS. 22a-c are bar graphs showing side-by-side comparisons of internalization, membrane binding and efflux over time of the same peptides seen in FIG. 21.

FIG. 23 is a comparison of internalization and efflux results obtained with ¹⁷⁷Lu-BRU-2813 in EFO-27 (ovarian cancer) and PC-3 (human prostate cancer) cell lines.

### DETAILED DESCRIPTION OF THE INVENTION

The present invention is directed to new peptide analogs of LHRH-II which have improved target binding affinity and/or improved metabolic stability over an iodinated prior art compound, Darg⁶,¹²⁵I-Tyr⁸,azaGly¹⁰-LHRH-II (¹²⁵I-LHRH-II). A number of changes can be made to the basic structure of LHRH-II, at the amino terminus, the carboxy terminus and/or at internal positions, with the resultant generation of LHRH-II analogs with enhanced target-binding affinity and/or enhanced resistance to proteolytic degradation. The analogs manifest these superior properties whether or not they are conjugated to a chelator and/or other component containing a detectable label. Furthermore, one of skill in the art would appreciate and expect that the scope of disclosed and exemplified substitutions at positions 1 and 2, for example, would make for effective and useful substitutions at those positions across the board, i.e., in unconjugated analogs, ones conjugated at the N-terminus and ones conjugated at the C-terminus.

Accordingly, one embodiment of the invention is a peptide of the formula

X₁-X₂-X₃-X₄-X₅-X₆-X₂-X₈-X₉-X₁₀,

wherein:
X₁ is an optional component which, when present, is selected from the group consisting of Arg, His, pGlu, Sar, Dnal2, Ac-Amfe4, Ac-Dnal2, Dtpi, Damfe4, Bip, Dbpa4, Tpi, Mogly, Ampha4, Dnal1, Qua3, Thy, Atdc2, Dtyr, Apsp, Hpgly, Datdc2 and Ahgly;
X₂ is selected from the group consisting of Arg, His, Gufe4, Damfe4, Ampg4, Darg and Ampa4;
X₃ is selected from the group consisting of Trp, Arg, Phe, Nal2, Nal1 and Amfe4;
X₄ is selected from the group consisting of Ser, Met, Asn, Amfe4 and Dap;
X₅ is selected from the group consisting of His, Arg, Orn and Fur3 ala;
X₆ is selected from the group consisting of Arg and Darg;
X₇ is Trp;
X₈ is selected from the group consisting of Bpa4, Tyr and Nal2;
X₉ is selected from the group consisting of Pro, Am2prd, Thz, Hypt4, Ampc4, Ampt4, Pip, Flp4 and Aze;
or X₈ and X₉ together can form a dipeptide isostere X₈-Ψ(CH₂N)—X₉; and
X₁₀ is an optional component which, when present, is selected from the group consisting of azaGly-NH₂, Gly-Arg-NH₂, Gly-Gln-NH₂, Da15o3t, Gua, Ap, Az34 m3buo-NH₂, Pheol, Mo2abn, A1gua5o3pt and Az23 m2po-NH₂;
with the proviso that the peptide is not pGlu-His-Trp-Ser-His-Darg-Trp-Tyr-Pro-azaGly-NH₂.

As disclosed herein, the analogs of the present invention may be conjugated to a component, or in some cases 2 components, containing a label detectable via any one of various known imaging means. Several embodiments of the invention along these lines may be defined as follows:

1) A peptide of the formula

X₁-X₂-X₃-X₄-X₅-X₆-X₇-X₈-X₉-linker-DL,
wherein X₁ through X₉ are as defined above and DL is a component containing a label detectable via scintigraphic imaging, magnetic resonance imaging, positron emission tomography imaging, single photon emission computed tomography imaging, a hand-held probe, ultrasound contrast analysis or optical imaging, or an enzymatically cleavable label.

2) A peptide of the formula

DL-optional linker-X₁-X₂-X₃-X₄-X₅-X₆-X₇-X₈-X₉-X₁₀,
wherein X₁ through X₁₀ are as defined above and DL is a component containing a label detectable via scintigraphic imaging, magnetic resonance imaging, positron emission tomography imaging, single photon emission computed tomography imaging, a hand-held probe, ultrasound contrast analysis or optical imaging, or an enzymatically cleavable label.

3) A peptide of the formula

DL₁-optional linker-X₁-X₂-X₃-X₄-X₅-X₆-X₇-X₈-X₉-X₁₀-linker-DL₂,
wherein X₁ through X₁₀ are as defined above; one of DL₁ and DL₂ is a chelator optionally complexed with a metal radionuclide; and the other is an optical imaging agent.

The doubly conjugated peptides in 3) above can be used for radiotherapeutic treatment of cancer or other LHRH mediated diseases, localization of tumors or LHRH binding sites, or both simultaneously.

Similarly, one of skill in the art would appreciate and expect that analogs of any of the family of LHRH isoforms containing substitutions according to the present invention would manifest the disclosed superior properties. Accordingly, another embodiment of the invention is an LHRH-analog peptide of the formula

X₁-X₂-X₃-Ser-X₅-Darg-X₇-X₈-Pro-azaGlyNH₂,

wherein:

X₁ is selected from the group consisting of Arg, His, pGlu, Sar, Dnal2, Ac-Amfe4, Ac-Dnal2, Dtpi, Damfe4, Bip, Dbpa4, Tpi, Mogly, Ampha4, Dnal1, Qua3, Thy, Atdc2, Dtyr, Apsp, Hpgly, Datdc2 and Ahgly;

X₂ is selected from the group consisting of Arg, His, Gufe4, Damfe4, Ampg4, Darg and Ampa4;

X₃ is selected from the group consisting of Trp and Tyr;

X₅ is selected from the group consisting of His, Leu and Tyr;

X₇ is selected from the group consisting of Leu and Trp; and

X₈ is selected from the group consisting of Bpa4 and Nal2, or the Bpa4 or Nal2 at position 8 can form a dipeptide Ψ(CH₂N) isostere with the Pro at position 9.

These LHRH-analog peptides may also be conjugated at the N- and/or C-terminus to a component containing a detectable label as set forth above.

Another aspect of the invention supported by the disclosure herein is a metabolically stabilized LHRH-II analog of the formula

X₁-X₂-Trp-Ser-His-X₆-Trp-X₈-X₉-GlyNH₂,

wherein:

X₁ is selected from the group consisting of pGlu, Dnal2 and Sar;

X₂ is Arg;

X₆ is Darg; X₈ is Bpa4; and

X₉ is selected from the group consisting of Pro, Am2prd, Thz, Hypt4, Ampc4, Ampt4, Pip, Flp4 and Aze; and

wherein when X₉ is Pro, it and the Bpa4 at position 8 together form a dipeptide Ψ(CH₂N) isostere.

These metabotically stabilized analogs may also be conjugated at the N- and/or C-terminus to a component containing a detectable label as set forth above.

Preferred examples of the analogs described herein are the peptides BRU-3103 (SEQ ID NO: 8), -3042 (SEQ ID NO: 9), -3102 (SEQ ID NO: 8), -2991 (SEQ ID NO: 8), -3045 (SEQ ID NO: 8), -3080 (SEQ ID NO: 8), -3044 (SEQ ID NO: 8), -3039 (SEQ ID NO: 8), -3043 (SEQ ID NO: 8), -3117 (SEQ ID NO: 10), -3041 (SEQ ID NO: 8), -3085 (SEQ ID NO: 8), -2992 (SEQ ID NO: 11), -2441 (SEQ ID NO: 12), -2734 (SEQ ID NO: 13), -3007 (SEQ ID NO: 8), -2439 (SEQ ID NO: 14), -2839 (SEQ ID NO: 15), -2803 (SEQ ID NO: 16), -2821 (SEQ ID NO: 17), -2822 (SEQ ID NO: 18), -3100 (SEQ ID NO: 19), -3115 (SEQ ID NO: 20), -3072 (SEQ ID NO: 21), -2964 (SEQ ID NO: 22), -3105 (SEQ ID NO:23), -2968 (SEQ ID NO: 24), -2969 (SEQ ID NO: 25), -3068 (SEQ ID NO: 26), -2959 (SEQ ID NO: 27), -3104 (SEQ ID NO: 28), -3111 (SEQ ID NO: 29), -2757 (SEQ ID NO: 30), -3058 (SEQ ID NO: 31), -2956 (SEQ ID NO: 32), -2952 (SEQ ID NO: 33), -2963 (SEQ ID NO: 34), -3070 (SEQ ID NO: 35), -3095 (SEQ ID NO: 36), -3081 (SEQ ID NO: 37), -3031 (SEQ ID NO: 38), -3050 (SEQ ID NO: 39), -3071 (SEQ ID NO: 40), -3053 (SEQ ID NO: 41), -3062 (SEQ ID NO: 42), -2813 (SEQ ID NO: 43), -2997 (SEQ ID NO: 44), -2796 (SEQ ID NO: 45), -3060 (SEQ ID NO: 46), -2961 (SEQ ID NO: 47), -2996 (SEQ ID NO: 48), -3094 (SEQ ID NO: 49), -2811 (SEQ ID NO: 50), -2869 (SEQ ID NO: 51), -3049 (SEQ ID NO: 52), -3027 (SEQ ID NO: 53), -3096 (SEQ ID NO: 54), -2993 (SEQ ID NO: 55), -3057 (SEQ ID NO: 56), -3069 (SEQ ID NO: 57), -3107 (SEQ ID NO: 58), -3055 (SEQ ID NO: 59), -2960 (SEQ ID NO: 60), -2984 (SEQ ID NO: 24), -2955 (SEQ ID NO: 61), -2995 (SEQ ID NO: 62), -3059 (SEQ ID NO: 63), -3098 (SEQ ID NO: 64), -3006 (SEQ ID NO: 24), -3054 (SEQ ID NO: 65), -3106 (SEQ ID NO: 66), -2696 (SEQ ID NO: 67), -2967 (SEQ ID NO: 68), -3056 (SEQ ID NO: 69), -3099 (SEQ ID NO: 70), -2797 (SEQ ID NO: 71), -2983 (SEQ ID NO: 24), -3020 (SEQ ID NO: 24), -3097 (SEQ ID NO: 72), -2985 (SEQ ID NO: 24), -2666 (SEQ ID NO: 73), -2962 (SEQ ID NO: 74), -3025 (SEQ ID NO: 75), -3063 (SEQ ID NO: 76), -2971 (SEQ ID NO: 77), -2876 (SEQ ID NO: 78), -3002 (SEQ ID NO: 79), -3021 (SEQ ID NO: 24), -2994 (SEQ ID NO: 80), -2953 (SEQ ID NO: 81) and -3122 (SEQ ID NO: 82). These peptides constitute examples of unconjugated, N-conjugated and C-conjugated analogs according to the invention. These peptides were tested and found to have superior binding affinity for LHRH binding sites on human ovarian cancer cells (EC₅₀≦0.5 nM) and/or enhanced metabolic stability. The structures of these peptides and other pertinent data can be found assembled in Table 26 near the end of the application.

The most preferred embodiments with respect to conjugated analogs are those bearing a chelator at either the N- or C-terminus Any chelator suitable for complexing with a metal ion or radionuclide can be used.

The metal chelators of the invention may include, for example, linear, macrocyclic, terpyridine, and N₃S, N₂S₂, or N₄ chelators (see also, U.S. Pat. No. 5,367,080, U.S. Pat. No. 5,364,613, U.S. Pat. No. 5,021,556, U.S. Pat. No. 5,075,099, U.S. Pat. No. 5,886,142, the disclosures of which are incorporated by reference in their entirety), and other chelators known in the art including, but not limited to, HYNIC, DTPA, EDTA, DOTA, TETA, and bisamino bisthiol (BAT) chelators (see also U.S. 5,720,934). For example, N₄ chelators are described in U.S. Pat. Nos. 6,143,274; 6,093,382; 5,608,110; 5,665,329; 5,656,254; and 5,688,487, the disclosures of which are incorporated by reference in their entirety. Certain N₃S chelators are described in PCT/CA94/00395, PCT/CA94/00479, PCT/CA95/00249 and in U.S. Pat. Nos. 5,662,885; 5,976,495; and 5,780,006, the disclosures of which are incorporated by reference in their entirety. The chelator may also include derivatives of the chelating ligand mercapto-acetyl-glycyl-glycyl-glycine (MAG3), which contains N₃S, and N₂S₂ systems such as MAMA (monoamidemonoaminedithiols), DADS (N₂S diaminedithiols), CODADS and the like. These ligand systems and a variety of others are described in Liu and Edwards, Chem. Rev. 1999, 99, 2235-2268 and references therein, the disclosures of which are incorporated by reference in their entirety.

The metal chelator may also include complexes containing ligand atoms that are not donated to the metal in a tetradentate array. These include the boronic acid adducts of technetium and rhenium dioximes, such as those described in U.S. Pat. Nos. 5,183,653; 5,387,409; and 5,118,797, the disclosures of which are incorporated by reference in their entirety.

Examples of preferred chelators include, but are not limited to, diethylenetriamine pentaacetic acid (DTPA), 1,4,7,10-tetraazacyclotetradecane-1,4,7,10-tetraacetic acid (DOTA), 1-substituted 1,4,7,-tricarboxymethyl 1,4,7,10-tetraazacyclododecane triacetic acid (DO3A), ethylenediaminetetraacetic acid (EDTA), 4-carbonylmethyl-10-phosphonomethyl-1,4,7,10-Tetraazacyclododecane-1,7-diacetic acid (Cm4 pm10d2a); and 1,4,8,11-tetraazacyclotetradecane-1,4,8,11-tetraacetic acid (TETA). Additional chelating ligands are ethylenebis-(2-hydroxy-phenylglycine) (EHPG), and derivatives thereof, including 5-Cl-EHPG, 5-Br-EHPG, 5-Me-EHPG, 5-t-Bu-EHPG, and 5-sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl-DTPA; bis-2 (hydroxybenzyl)-ethylene-diaminediacetic acid (HBED) and derivatives thereof; the class of macrocyclic compounds which contain at least 3 carbon atoms, more preferably at least 6, and at least two heteroatoms (O and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7, 10-tetra(methyl tetraacetic acid), and benzo-TETMA, where TETMA is 1,4,8,11-tetraaza-cyclotetradecane-1,4,8,11-(methyl tetraacetic acid); derivatives of 1,3-propylenediamine-NEWYORK 7522738 (2K) tetraacetic acid (PDTA) and triethylenetetraaminehexaacetic acid (TTHA); derivatives of 1,5,10-N,N′,N″-tris(2,3-dihydroxybenzoyl)-tricatecholate (LICAM) and 1,3,5-N,N′,N″-tris-(2,3-dihydroxybenzoyl) aminomethylbenzene (MECAM). Other preferred chelators include Aazta and derivatives thereof including CyAazta. Examples of representative chelators and chelating groups contemplated by the present invention are described in WO 98/18496, WO 86/06605, WO 91/03200, WO 95/28179, WO 96/23526, WO 97/36619, PCT/US98/01473, PCT/US98/20182, and U.S. Pat. No. 4,899,755, U.S. Pat. No. 5,474,756, U.S. Pat. No. 5,846,519 and U.S. Pat. No. 6,143,274, each of which is hereby incorporated by reference in its entirety.

Another class of chelators that can be used in the practice of the invention includes such species as N,N-dimethylGly-Ser-Cys; N,N-dimethylGly-Thr-Cys; N,N-diethylGly-Ser-Cys; N,N-dibenzylGly-Ser-Cys; and other variations thereof. For example, spacers which do not actually complex with the metal radionuclide, such as an extra single amino acid Gly, may be attached to these metal chelators (e.g., N,N-dimethylGly-Ser-Cys-Gly; N,N-dimethylGly-Thr-Cys-Gly; N,N-diethylGly-Ser-Cys-Gly; N,N-dibenzylGly-Ser-Cys-Gly). Other useful metal chelators are such as all of those disclosed in U.S. Pat. No. 6,334,996, also incorporated by reference (e.g., Dimethylgly-L-t-Butylgly-L-Cys-Gly; Dimethylgly-D-t-Butylgly-L-Cys-Gly; Dimethylgly-L-t-Butylgly-L-Cys, etc.).

The class of chelators known as PnAO chelators, such as are disclosed in U.S. Pat. No. 5,808,091; U.S. Pat. No. 6,184,361; U.S. Pat. No. 5,688,487; U.S. Pat. No. 6,359,120; U.S. Pat. No. 6,699,458; and U.S. Pat. No. 6,958,141, and heteroatom-bridged bis amine bis oxime ligands (e.g. oxa PnAO chelators) that are disclosed in U.S. Pat. No. 5,608,110; U.S. Pat. No. 5,627,286; U.S. Pat. No. 5,665,329; U.S. Pat. No. 5,656,254; and U.S. Pat. No. 5,741,912 may also be used in the practice of the invention. These disclosures are hereby incorporated by reference in their entirety.

The preferred chelators to be used are selected from DO3A10CM, DTPA, NOTA, PnAO, oxa PnAO and N,N-dimethyl-Gly-Ser-Cys. The most preferred chelator is DO3A10CM.

The chelators are optionally, and preferably, complexed with an appropriate metal radionuclide. Preferred metal radionuclides for scintigraphy or radiotherapy include ^{99m}Tc, ⁵¹Cr, ⁶⁷Ga, ⁶⁸Ga, ⁴⁷Sc, ⁵¹Cr, ¹⁶⁷Tm, ¹⁴¹Ce, ¹¹¹In, ¹⁶⁸Yb, ¹⁷⁵Yb, ¹⁴⁰La, ⁹⁰Y, ⁸⁸Y, ¹⁵³Sm, ¹⁶⁶Ho, ¹⁶⁵Dy, ¹⁶⁶Dy, ⁶²Cu, ⁶⁴Cu, ⁶⁷Cu, ⁹⁷Ru, ¹⁰³Ru, ¹⁸⁶Re, ²⁰³Pb, ²¹¹Bi, ²¹²Bi, ²¹³Bi, ²¹⁴Bi, ²²⁵Ac, ¹⁰⁵Rh, ¹⁰⁹Pd, ^{117m}Sn, ¹⁴⁹Pm, ¹⁶¹Tb, ¹⁷⁷Lu, ¹⁹⁸Au and ¹⁹⁹Au. The choice of metal will be determined based on the desired therapeutic or diagnostic application. For example, for diagnostic purposes the preferred radionuclides include ⁶⁴Cu, ⁶⁷Ga, ⁶⁸Ga, ^{99m}Tc, and ¹¹¹In. For therapeutic purposes, the preferred radionuclides include ⁶⁴Cu, ⁹⁰Y, ¹⁰⁵Rh, ¹¹¹In, ^{117m}Sn, ¹⁴⁹Pm, ¹⁵³Sm, ¹⁶¹Tb, ¹⁶⁶Dy, ¹⁶⁶Ho, ¹⁷⁵Yb, ¹⁷⁷Lu, ^{186/188}Re, and ¹⁹⁹Au. Depending on the radionuclide employed, the conjugated peptides can be used for radiotherapeutic purposes, diagnostic purposes or both.

The radiolabeled peptides can be prepared by methods known to those skilled in the art, and stabilized against radiolytic damage using, for example, the methods disclosed in US 2007/0269375 and in WO 05/009393, both of which are hereby incorporated by reference in their entirety.

For peptides conjugated to a chelator at the N-terminus, a linker connecting the peptide and chelator is optional; for peptides conjugated to a chelator at the C-terminus, a linker is required for optimal utility. The linkers may be selected from any suitable moieties, taking into account the different chemical requirements for binding to the N- and C-termini. When employed, preferred linkers at the N-terminus are selected from the group consisting of Da48oa, Amb4, Gly, Dap, Gly-Abz4, Lys and Dlys. Preferred linkers to be used at the C-terminus are selected from the group consisting of Dae, Dabt14, Ampip2, Da15o3pt, Maz4dahp17, Bampy 26, Bap14p, Da18o36oc and Dapt15.

The component to which the peptide analog may be conjugated is by no means confined to a chelator; any component containing a detectable label may be employed. The detectable label is any moiety whose presence can be monitored by an imaging procedure or otherwise detected (e.g. with a hand-held probe); in other words, the moiety is able in any way to provide, to improve or to advantageously modify the signal detected. Such techniques include, but are not limited to, scintigraphic imaging, magnetic resonance imaging (MRI), positron emission tomography (PET) imaging, ultrasound imaging, optical imaging or imaging via monitoring of an enzymatically cleavable label, or detection with a hand-held probe.

Another aspect of the present invention relates to modifications of the foregoing peptides to provide LHRH binding site-specific imaging agents by conjugation to a detectable label. For example, peptides of the invention conjugated to a radiolabel, an enzymatic label, a color-generating label, a label detectable by MRI, such as MR paramagnetic chelates or microparticles; conjugated to or incorporated into an ultrasound contrast agent such as gas-filled microvesicles (e.g. microbubbles, microparticles, microspheres, emulsions, or liposomes); or conjugated to an optical imaging agent, including an optical dye, would be such compounds. Such conjugated peptides according to the present invention are useful in any application where binding, detecting or isolating LHRH binding sites (e.g. on tumors) is advantageous.

Examples of detectable labels or diagnostically effective moieties according to the invention include, for instance, chelated gamma ray or positron emitting radionuclides; paramagnetic metal ions in the form of chelated or polychelated complexes, X-ray absorbing agents including atoms having atomic number higher than 20; an ultrasound contrast agent, including, for example, a gas-filled microvesicle; a molecule absorbing in the UV spectrum; a quantum dot; a molecule capable of absorption within near or far infrared radiations; any one of many optical labels known in the art; and, in general, any moiety which generates a detectable substance.

In another preferred embodiment, the analogs of the invention that bind to the LHRH binding site may be conjugated (directly or via a linker) to an optically active imaging moiety. Suitable examples of optically active imaging moieties include, for example, optical dyes, including organic chromophores or fluorophores, having extensive delocalized ring systems and having absorption or emission maxima in the range of 400-1500 nm; fluorescent molecules such as fluorescein; phosphorescent molecules; bioluminescent molecules; light-absorbing molecules; and light-reflecting and -scattering molecules.

In accordance with the present invention, a number of optical parameters may be employed to determine the location of LHRH binding sites (e.g. on tumors) with in vivo light imaging after introduction to the subject of an optically-labeled moiety of the invention. Optical parameters to be detected in the preparation of an image may include transmitted radiation, absorption, fluorescent or phosphorescent emission, light reflection, changes in absorbance amplitude or maxima, and elastically scattered radiation. For example, biological tissue is relatively translucent to light in the near infrared (NIR) wavelength range of 650-1000 nm. NIR radiation can penetrate tissue up to several centimeters, permitting the use of the moieties of the present invention for optical imaging of LHRH binding sites in vivo.

Near infrared dyes may include cyanine or indocyanine derivatives such as, for example, Cy5.5, IRDye800, indocyanine green (ICG), indocyanine green derivatives including the tetrasulfonic acid substituted indocyanine green (TS-ICG), and combinations thereof.

After introduction of the optically-labeled moiety of the invention, the patient is scanned with one or more light sources (e.g., a laser) in the wavelength range appropriate for the photolabel employed in the agent. The light used may be monochromatic or polychromatic and continuous or pulsed. Transmitted, scattered, or reflected light is detected via a photodetector tuned to one or multiple wavelengths to determine the location of LHRH binding sites such as tumors in the subject. Changes in the optical parameter may be monitored over time to detect accumulation of the optically-labeled reagent at the LHRH binding site. Standard image processing and detecting devices may be used in conjunction with the optical imaging reagents of the present invention.

Additionally, the binding peptides of the invention may be attached to an enzyme substrate that is linked to both a light-imaging reporter and a light-imaging quencher. The binding moiety serves to localize the construct to the LHRH binding site-bearing tissue of interest, where an enzyme cleaves the enzyme substrate, releasing the light-imaging quencher and allowing light imaging of the tissue of interest.

The peptides of the invention also may be conjugated with a radionuclide reporter appropriate for PET imaging. For use as a PET agent, a peptide according to the invention is complexed (optionally via a chelator) with one of the various positron-emitting metal ions, such as ⁵¹Mn, ⁵²Fe, ⁶⁰Cu, ⁶⁸Ga, ⁷²As, ^{94m}Tc, or ¹¹⁰In.

Still another embodiment of the invention is a peptide of the formula

X₁-X₂-X₃-X₄-X₅-X₆-X₇-X₈-X₉-X₁₀,
wherein X₁ through X₁₀ are as defined above; and wherein one of X₁ through X₁₀, or an additional residue X₁₁ bound either to X₁ or X₁₀, is labeled with a radioisotope selected from the group consisting of ¹²³I, ¹²⁴I, ¹²⁵I, and ¹³¹I.

In such peptides, a useful radioisotope of a nonmetal, iodine, can be introduced directly via iodination of a suitable amino acid residue which is either already a part of the primary peptide structure or is added to either end of the primary peptide via standard procedures for peptide synthesis. The iodination is most commonly, but not necessarily, achieved on a tyrosine residue. When position 1 of the peptide is occupied by Dtyr, that would, for example, also be a good iodination site. Methods for introducing iodine and other halogens into a molecule are known to those skilled in the art (see, e.g., Wilbur, D. S. Bioconjugate Chemistry 1982, 3, 433-470). Methods include the use of halogen oxidizing reagents such as chloramine T or Iodogen, the use of oxidizing enzymes such as lactoperoxidase, use of aryl diazonium-containing intermediates, organomercury, organoborate and organostannane derivates and the addition of a radiohalogenated conjugate such as Bolton-Hunter reagent. Depending on the isotope introduced, the peptide can be used in radiotherapy, scintigraphic imaging or both. ¹²⁵I and ¹³¹I are therapeutically useful isotopes; and ¹²³I, ¹²⁴I and ¹³¹I render the peptides useful as imaging tools. This embodiment can also be practiced by introduction of an alternate halogen radionuclide such as ¹⁸F, ⁷⁶Br or ⁷⁷Br, instead of an iodine radionuclide, using methods known to those skilled in the art, e.g., the methods described by P. W. Miller et al. Angew. Chem. Int. Ed. Engl. 2008, 47(47), 8998-9033.

The unconjugated peptides of the invention are useful in targeted therapy of cancers or other LHRH-mediated diseases, in particular prostate, ovarian and breast cancers. Peptides conjugated at either the N-terminus or C-terminus with a radionuclide-complexed chelator can be used in targeted radiotherapy, targeted imaging or both, depending on the radionuclide involved. Peptides conjugated at either terminus with another component (other than a chelator) containing a detectable label are useful in targeted imaging.

Accordingly, the present invention is also directed to methods employing the various novel peptide analogs, as appropriate, for targeted therapy of sex-hormone-related cancers, in particular prostate, ovarian and breast cancers.

The invention is directed still further to methods employing the novel peptide analogs, as appropriate, for targeted radiotherapy of sex-hormone-related cancers, in particular prostate, ovarian and breast cancers.

The invention is also concerned with methods employing the novel peptide analogs, as appropriate, for targeted imaging in patients. More particularly, the methods involve localizing LHRH binding sites, such as tumors, and/or evaluating the potential for treatment of a patient, particularly a patient with prostate, ovarian or breast cancer.

Although certain conditions have been set forth as the primary ones that would be amenable to treatment according to the present invention, it will be appreciated that the inventive peptides have credible potential usefulness in the treatment of any and all disorders related to the LHRH-gonadotropin system. Further examples of such disorders are endometriosis, uterine fibroids, benign prostate hyperplasia, fertility disorders and precocious puberty.

In conjunction with the methods of treatment and imaging described herein, the invention is also concerned with pharmaceutical compositions comprising the inventive peptide analogs (conjugated or not) and pharmaceutically acceptable carriers. The carriers may be selected from any of the diluents, excipients and other carriers well known to those of skill in the pharmaceutical art. Virtually any mode of administration may be used in the practice of the invention. Among the modes particularly envisioned are intravenously, intranasally, orally and intramuscularly.

### ABBREVIATIONS

The following abbreviations have been used:

aa/AA=Amino acid

### ACN=Acetonitrile

Adoa=8-Amino-3,6-dioxaoctanoic acid
API-ES=Atmospheric pressure ionization electrospray
AzaG-NH₂/AzaGly-NH₂=Azaglycine amide

### Bn=Benzyl

Boc=t-Butyloxycarbonyl

### Bpa4=(L)-4-Benzoylphenylalanine

### Bu=Butyl

### C/Cys=(L)-Cysteine

### Cbz=Benzyloxycarbonyl

### CDI=1,1′-Carbonyldiimidazole

### DCM=Dichloromethane

### DIC=N,N′-Diisopropylcarbodiimide

### DIEA=N,N-Diisopropylethylamine

### Dlys=(D)-Lysine

### DMF=N,N-Dimethylformamide

DMSO=Dimethyl sulfoxide

### Dnal2=(D)-2-Naphthylalanine

DO3A10CM(tris-t-butyl)=2-[1,4,7,10-tetraaza-4,7,10-tris(3,3-dimethyl-2-oxobutyl)cyclododecyl]acetic acid

### Dtyr=(D)-Tyrosine

ee=Enantiomeric excess
Et₂O=Diethyl ether
EtOAc=Ethyl acetate

### F/Phe=(L)-Phenylalanine

### Fmoc=9-Fluorenylmethoxycarbonyl

### G/Gly=Glycine

### H/His=(L)-Histidine

HATU=2-(1H-7-Azabenzotriazol-1-yl)-1,1,3,3-tetramethyluronium hexafluorophosphate
HBTU=2-(1H-Benzotriazole-1-yl)-1,1-3,3-tetramethylaminium hexafluorophosphate
HFIPA=1,1,1,3,3,3-Hexafluoroisopropyl alcohol
HOAc=Acetic acid
HOBt.H₂O=N-Hydroxybenzotriazole monohydrate

### IBCF=Isobutylchloroformate

### K/Lys=(L)-Lysine

### L/Leu=(L)-Leucine

### Lu=Lutetium

### M/Met=(L)-Methionine

### MeOH=Methanol

NaOAc=Sodium acetate
Neg. ion=Negative ion

### NHS=N-Hydroxysuccinimide

### NMM=N-Methylmorpholine

### NMP=N-Methylpyrrolidine

### P/Pro=(L)-Proline

Pd/C=Palladium-on-carbon catalyst
PET=Positron emission tomography
Pbf=2,2,4,6,7-Pentamethyl-2,3-dihydrobenzo[b]furan-5-sulfonyl
pGlu=Pyroglutamic acid
Pmc=2,2,5,7,8-Pentamethylchroman-6-sulfonyl
Pos. ion=Positive ion
PyBop=Benzotriazole-1-yl-oxy-tris-pyrrolidino-phosphonium hexafluorophosphate

### Q/Gln=(L)-Glutamine

### R/Arg=(L)-Arginine

r/Darg=(D)-Arginine
RCP=Radiochemical purity
Reagent A=95:25:2.5, TFA:H₂O:TIPS (v/v/v)
Reagent B=88:5:5:2, TFA:H₂O:phenol:TIPS (v/v/w/v)
RT=Room temperature

### S/Ser=(L)-Serine

### Sar=Sarcosine, N-methylglycine

SPECT=Single photon emission computed tomography
SPPS=Solid-phase peptide synthesis

### Su=Succinimidyl

TFA=Trifluoroacetic acid

### TFE=2,2,2-Trifluoroethanol

### THF=Tetrahydrofuran

### TIPS=Triisopropylsilane

t_{R}=Retention time (minutes)

### Trt=Trityl

### W/Trp=(L)-Tryptophan

### Y/Tyr=(L)-Tyrosine

Names, structures and abbreviations of linkers, amines and unusual/unnatural amino acids used in the synthesis of various LHRH-II analog peptides are provided in Tables 14, 16 and 20.

### Reagents and Analytical Methods for Synthesized Peptides

Solvents for reactions, chromatographic purification and HPLC analyses were E. Merck Omni grade solvents from VWR Corporation (West Chester, Pa.). N-Methylpyrrolidinone (NMP) and N,N-dimethylformamide (DMF) were purchased from Pharmco Products Inc. (Brookfield, Conn.), and were peptide synthesis grade or low water/amine-free Biotech grade quality. Piperidine (sequencing grade, redistilled 99+%) and trifluoroacetic acid (TFA) (Spectrophotometric grade or sequencing grade) were purchased from Sigma-Aldrich Corporation (Milwaukee, Wis.) or from the Fluka Chemical Division of Sigma-Aldrich Corporation. Phenol (99%), N,N-diisopropylethylamine (DIEA), N,N-diisopropylcarbodiimide (DIC) and triisopropylsilane (TIS) were purchased from Sigma-Aldrich Corporation. Fmoc-protected amino acids, PyBop, HBTU and 1-hydroxybenzotriazole (HOBt) were purchased from Nova-Biochem (San Diego, Calif., USA), Advanced ChemTech (Louisville, Ky., USA), Chem-Impex International (Wood Dale Ill., USA), and Multiple Peptide Systems (San Diego, Calif., USA). Fmoc-8-amino-3,6-dioxaoctanoic acid (Adoa) was obtained from NeoMPS Corp (San Diego, Calif.) or Suven Life Sciences (Hyderabad, India).

Solvents suitable for peptide synthesis were purchased from Pharmco-AAPER. Resins used in the solid phase synthesis were procured either from Novabiochem and/or Chemlmpex Intl. Protected amino acids were obtained from Novabiochem, Chemlmpex Intl. and Advanced Chem. Tech. Other solvents and chemicals were purchased from Sigma-Aldrich and Alfa Aesar.

Preparative HPLC was conducted on a Shimadzu LC-8A dual pump gradient system equipped with an SPD-10AV UV detector fitted with a preparative flow cell and controlled by Shimadzu Class VP version 4.3 software. Generally the solution containing the crude peptide was loaded onto a reversed-phase C18 column, using a third pump attached to the preparative Shimadzu LC-8A dual pump gradient system. After the solution of the crude product mixture was applied to the preparative HPLC column, the reaction solvents and solvents employed as diluents, such as DMF or DMSO, were eluted from the column at low organic phase composition. Then the desired product was eluted using a gradient elution of eluent B into eluent A. Product-containing fractions were combined based on their purity as determined by analytical HPLC and mass spectral analysis. The combined fractions were freeze-dried to provide the desired product.

Analytical HPLC data were generally obtained using a Shimadzu LC-10AT VP dual pump gradient system employing a Waters XTerra MS-C18 4.6×50 mm column, (particle size: 5μ; 120A pore size) and gradient or isocratic elution systems using water (0.1% TFA) (v/v) as eluent A and CH₃CN (0.1% TFA) (v/v) as eluent B. Detection of compounds was accomplished using UV at 220 and 230 nm.

Mass spectral data were obtained in-house on an Agilent LC-MSD 1100 Mass Spectrometer. For the purposes of fraction selection and characterization of the products, mass spectral values were usually obtained by API-ES with a Model G1987 multimode ionization source in positive ion mode. Generally the molecular weight of the target peptides was ˜2000; the mass spectra usually exhibited strong doubly or triply positively-charged ion-mass values rather than weak [M+H]⁺. These were generally employed for selection of fractions for collection and combination to obtain the pure peptide during HPLC purification.

### General Methods for Solid-Phase Peptide Synthesis (SPPS)

The linear peptides were synthesized using an established automated protocol on a Rainin PTI Symphony® Peptide Synthesizer (twelve peptide sequences/synthesis) using Fmoc-PAL-PEG-PS resin (0.2 mmol/g), Fmoc-protected amino acids and PyBop-mediated ester activation in DMF. The PAL-PEG-PS resin preloaded with Fmoc-Pro-azaGly (substitution level 0.2 mmol/g) was used for synthesis. The rest of the peptide sequence was loaded on the Fmoc-Pro-azaGly-PAL-PEG-PS resin in stepwise fashion by SPPS methods, typically on a 50 mmol scale. The amino acid coupling was carried out with a 4-fold excess each of amino acid and PyBop-DIEA reagent in DMF.

In a typical amino acid coupling process, 1.25 mL of DMF containing 200 mmol of an amino acid, followed by PyBOP (200 mmol, DMF solution, 1.25 mL) and DIEA (200 mmol, DMF solution, 1.25 mL) were added in succession by an automated protocol to a reaction vessel containing the resin (50 mmol) which was agitated by recurrent nitrogen bubbling. After 1 h coupling time, the resin was washed thoroughly with DMF (6×4.5 mL) and the cleavage of the Fmoc-group was performed with 25% piperidine in DMF (4.5 mL) for 10 min, followed by a second treatment with the same reagent for 10 min to ensure complete deprotection. Again, the resin was thoroughly washed with DMF (5 mL/g, 6×) interposed with a CH₂CH₂ (10 mL/g) wash in between DMF washes. This guaranteed that the resin was free from the residual piperidine and ready for the ensuing amino acid coupling.

To introduce the N-substituted glycine at position AA¹ during solid phase synthesis, appropriate intervening coupling protocols during sequence build-up on the resin were introduced which involved the submonomer peptoid coupling technique.⁴³ First, bromoacetic acid (4 eq.) was coupled instead of Gly using N,N-diisopropylcarbodiimide (DIC, 4 eq.) in DMF as coupling agent. This was followed by the alkylation reaction on the resin-bound bromoacetamide with the corresponding primary amine (20 eq. for 4 h) in DMF (5.0 mL) to create the N-substituted glycine moiety at position 1 in the sequence on the resin. In general, 8 h coupling time was employed for coupling of Fmoc-AA-OH to a secondary amino group on the resin. The duration of the final coupling of DOTA-tris-t-butyl ester to a primary/secondary amino group on the resin was extended to 18 h. After completion of the peptide synthesis, the resin was subjected to a cleavage protocol on the synthesizer with the cleavage cocktail, “reagent B” (TFA:water:phenol:triisopropylsilane, 88:5:5:2, v/v/w/v) (10 mL/g of resin) for 4 h. Cleavage solutions containing peptides were evaporated under vacuum to remove volatiles. The paste thus obtained in each case was triturated with ether to provide a solid which was pelleted by centrifugation, followed by 3 more cycles of ether washing and pelleting. The resulting solid was dried under vacuum to obtain the crude peptide as an off-white solid. A 50-μmol scale synthesis of a peptide of MW ˜1900 gave 100 mg (105% of theory) of the crude peptide. The greater than theoretical yield is most likely due to the inconsistency in the loading level/weighing of the resin or due to moisture and residual solvents.

### Purification of LHRH-II Peptides—General Procedure

A 50-1.μmol scale synthesis of a LHRH peptide of MW ˜1900 on the ‘Symphony’ instrument provided ˜100 mg of crude peptide from each reaction vessel (RV). Since the reversed-phase C18 preparative HPLC column (50×250 mm) employed for purification of peptides is capable of purifying about 0.2 g of crude peptide/injection, all of the crude peptide (˜100 mg) was purified in a single run. The crude peptide (˜100 mg) dissolved in CH₃CN (10 mL) was diluted to a final volume of 50 mL with water and the solution was filtered. The filtered solution was loaded onto the preparative HPLC column (Waters, Xterra® Prep MS C₁₈, 10μ, 120 Å, 50×250 mm) which had been pre-equilibrated with 10% CH₃CN in water (0.1% TFA). During the application of the sample solution to the column the flow of the equilibrating eluent from the preparative HPLC system was stopped. After the sample solution was applied to the column, the flow of equilibrating eluent from the gradient HPLC system was reinitiated and the composition of the eluent was then ramped to 20% CH₃CN-water (0.1% TFA) over 1 min after which a linear gradient at a rate of 0.5%/min of CH₃CN (0.1% TFA) into water (0.1% TFA) was initiated and maintained for 50 min. Fractions (15 mL) were manually collected using UV at 220 nm as an indicator of product elution. The collected fractions were analyzed on an analytical reversed-phase C18 column (Waters Xterra MS-C18, 5μ, 120 Å, 4.6×50 mm) and product-containing fractions of >95% purity were combined and freeze-dried to afford the corresponding LHRH peptide. Typically the purification of 100 mg of crude peptide afforded 10 to 15 mg (10 to 15% yield) of the desired LHRH peptide (>95% purity). After isolation, the peptides were analyzed by HPLC and mass spectrometry to confirm identity and purity.

### LHRH-II Analogs Bearing a Detectable Label (e.g. the Chelator DO3A10CM) at the N-Terminus

One of the goals was to explore new LHRH derivatives based on LHRH-II that could be derivatized with detectable labels such as radiometals, as such compounds could potentially be used for diagnostic imaging or for targeted radiotherapy. For example, imaging using LHRH receptor-targeted compounds conjugated to a detectable label or radiotherapeutic isotope might help to localize LHRH binding sites and/or be useful to evaluate the potential for radiotherapeutic treatment of patients with receptor-positive tumors. A variety of radionuclides are useful for radioimaging including ⁶⁷Ga, ⁶⁸Ga, ^{99m}Tc, ¹¹¹In, ¹²³I, ¹²⁴I and ¹⁸F, while isotopes such as ¹⁸⁶Re, ¹⁸⁸Re, ⁶⁷Cu, ¹⁸⁸Re, ⁹⁰Y, ¹¹¹In and ¹⁷⁷Lu can be used for radiotherapy. Most of these radionuclides must be bound via a chelating agent.

Detectable labels or metal chelating agents such as the monosubstituted DO3A derivative DO3A10CM can be introduced into peptide side chains by means of site-selective reactions involving particular amino acid residues. For example the lysine residue at position 6 of LHRH analogs has been directly acylated with a metal chelating group.⁴² Alternatively, a metal-binding ligand or other detectable label can be added to the N-terminus of a peptide. Placing the detectable label/chelating moiety on the N-terminus of the peptide rather than on an amino acid in the middle of the peptide has the added advantage of spatially distancing the detectable label, such as a metal complex, from the peptide core backbone, thereby minimizing the effect of the label on the peptide conformation.

The synthesis of various analogs of LHRH-II with DO3A10CM at the N-terminus and binding studies with these constructs on human ovarian cancer cells (EFO-27) were carried out to determine the effect of systematic changes in peptide sequence on binding affinity. The compounds may prove suitable for imaging studies and/or for the delivery of radiotherapeutic isotopes of metals like ¹⁷⁷Lu. The studies were performed with a particular view to developing structure-function studies for the development of a ¹⁷⁷Lu-LHRH-based radiotherapeutic agent to treat human ovarian cancer but also more generally to develop LHRH analogs with potential as radiotherapeutic and radioimaging agents in the diagnosis and treatment of sex-hormone-related diseases and cancers.

Based on literature reports that LHRH analogs with azaglycine at position 10 provided peptides that are more stable to chorionic post-proline peptidase enzyme degradation^{44,45} and have a longer duration of biological action, LHRH-II sequences with azaglycine at position 10 were selected for synthesis. Likewise, it was known that highly active analogs of LHRH peptides can be obtained by replacing Gly⁶ with a D-amino acid and the glycine amide residue at position 10 with various alkylamides.⁴⁶ These data indicated that a good starting point for synthetic efforts was DO3A10CM-Sar-His-Trp-Ser-His-Darg-Trp-Tyr-Pro-azaGly-NH₂ (BRU-2440, seq004 (SEQ ID NO: 83)).

A solid-phase peptide synthesis (SPPS) method for the preparation of LHRH-II analogs and rigorous HPLC purification methods to obtain peptides in high purity were developed. This methodology allowed the preparation of these analogs with a metal chelating agent, DO3A10CM at the N-terminus of the peptide sequence and facilitated the suitable substitution of various lipophilic and hydrophilic amino acids in the sequence, and at the C-terminus with various alkylamines or oxyalkylamines. All these analogs (nearly 200 in total) were tested for specific in vitro binding to human ovarian cancer EFO-27 cells and their relative activities were determined. Based on the EC₅₀ data, assessment of the structure-function relationship of these LHRH-II analogs was performed.

The replacement of azaGly at position 10 with oxyalkylamines, insertion of Darg at position 6 and diverse substitution of basic lipophilic amino acids, especially with arginine or Dnal2, at positions 1 and 2 were emphasized in the development study of an LHRH-analog with high potency in vitro. LHRH-II analogs with acidic amino acids showed much decreased potency indicating that the —COOH group was not tolerated. An agonist (EC₅₀=0.14 μM) containing a diamino acid with a distant amino group as linker between DO3A10CM and N-terminus, and Pro⁹-oxyalkylamide, indicated that based on the requirement of basic lipophilic amino acids and chain length, it could be possible to fine tune the character of the peptide by the inclusion of appropriate basic unnatural amino acids and modification in the total chain length and thereby to develop a highly potent analog.

The analog peptides bearing a chelator at the N-terminus were synthesized and purified, and the effects of substitutions at various positions were assessed in terms of binding affinities as set forth below.

### Loading of Fmoc-Pro-azaGly on Fmoc-PAL-PEG-PS Resin

Removal of the Fmoc group of the pre-soaked/swelled (DMF) Fmoc-PAL-PEG-PS resin (50 g, 10.00 mmol, 0.2 mmol/g) was performed in a peptide synthesis flask with 25% piperidine in DMF (250 mL) for 10 min, followed by a second treatment with 25% piperidine in DMF (250 mL) for 10 min to ensure complete deprotection. The resin was then thoroughly washed with DMF (6×250 mL). N,N-Carbonyldiimidazole (16.20 g, 100.0 mmol, 10 eq.) was added to the suspension of the resin in DMF (200 mL) and the resin was agitated for 4 h. The reaction solution was drained from the flask and the resin was washed with DMF (2×200 mL). Hydrazine.hydrate (2.0 g, 40.0 mmol, 4 eq.) in DMF (200 mL) was added to the resin. After agitating the resin for 8 h, the reaction solution was drained and the resin was washed thoroughly with DMF (6×200 mL). Fmoc-Pro-OH (13.5 g, 40.0 mmol), PyBOP (15.18 g, 40.0 mmol) and DIEA (10.32 g, 80 mmol) were added sequentially to the suspension of the resin in DMF (200 mL) and the resin was agitated for 4 h. After coupling of Fmoc-Pro-OH, the resin was washed with DMF (200 mL×2) followed by washing with CH₂Cl₂ (4×200 mL) and dried under vacuum. The resin loading was determined by treatment of a small aliquot of the dry resin (5 mg) with piperidine followed by the spectrophotometric analysis⁴⁷ of the piperidine-fulvene adduct in DMF solution. The resin load of Fmoc-Pro-azaGly was found to be 0.19 mmol/g.

Preparation of BRU-2907 from BRU-2443

NH₂OH (0.56 mmol) in methanol was prepared by neutralizing NH₂OH.HCl (3.89 g, 0.56 mmol) with NaOH (2.24 g, 0.56 mmol) in methanol (10 mL) at 0° C. Solid NaCl was removed by filtration and BRU-2443 (50 mg, 0.028 mmol) was added, followed by stirring at 40° C. for 4 h. The reaction mixture was diluted to 100 mL with water and then purified via reversed-phase C18 HPLC chromatography following the general procedure for purification to isolate pure BRU-2907; Yield: 25 mg (50%).

Synthesis of LHRH-II Analogs with Modification at Position 10 (AA¹⁰)
1. DO3A10CM-Dnal2-Arg-Trp-Ser-His-Darg-Trp-Bpa4-Pro-Az34 m3buo-NH₂ (BRU-2967)

Fmoc-PAL-PEG-PS resin (0.22 mol/g, 1.14, 0.25 mmol) was swelled with 15.0 mL of DMF for 15 min in a manual peptide synthesis vessel and the solution was drained. The protecting group was removed to expose the amine on the resin using Protocol C. A solution of bromoacetic acid (0.139 g, 1.0 mmol) in DMF (5.0 mL) was activated with HOBt.H₂O (0.153 g, 1.0 mmol) and DIC (0.139 g, 1.1 mmol) and transferred to the suspension of the resin in 10.0 mL of DMF and the peptide vessel was agitated for 20 h. The resin was drained and washed with 3×15 mL of DMF. N-Methylhydrazine (0.46 g, 10.0 mmol) in DMF (15.0 mL) was added to the resin and the resin was agitated for 4-6 h at ambient temperature. The reaction solution was drained and the resin was washed with 3×15 mL of DMF. Activated Fmoc-Pro-OH (refer to Protocol H) was coupled to the hydrazino amide on the resin. The rest of the sequence was constructed employing Protocols A, D, L and purified using Protocol E. Yield: 14.5 mg (3%).

The peptides listed below were also prepared. Yield: mg (% yield; protocols employed).
2. DO3A10CM-Dna12-Arg-Trp-Ser-His-Darg-Trp-Bpa4-Pro-Da15o3pt (BRU-2968). Yield: 16.4 mg(3.4%; A, D, G, L and E)
3. DO3A10CM-Dna12-Arg-Trp-Ser-His-Darg-Trp-Bpa4-Pro-Gly-Arg-NH₂ (BRU-2969). Yield: 24.2 mg (4.7%; A, C, D, I and E)
4. DO3A1-CM-Dna12-Arg-Trp-Ser-His-Darg-Trp-Bpa4-Pro-Ach2 (BRU-2970) (SEQ ID NO: 24). Yield: 22.0 mg (4.6%; A, C, D, G, L and E)
5. DO3A10CM-Dna12-Arg-Trp-Ser-His-Darg-Trp-Bpa4-Pro-Gly-Gln-NH₂ (BRU-2971). Yield: 29.0 mg(5.7%, A, C, D, L and E)
6. DO3A100CM-Dna12-Arg-Trp-Ser-His-Darg-Trp-Bpa4-Pro-Gly-Abt1h4 (BRU-2978) (SEQ ID NO: 84). Yield: 18.0 mg (11.5%. A, C, D, G, L and E)
7. DO3A10CM-Dna12-Arg-Trp-Ser-His-Darg-Trp-Bpa4-Pro-Gly-Abn (BRU-2979) (SEQ ID NO: 84. Yield: 36.5 mg (23%, A, C, D, G, L and E)
8. DO3A10CM-Dna12-Arg-Trp-Ser-His-Darg-Trp-Bpa4-Pro-Gly-Phe-NH₂ (BRU-2980) (SEQ ID NO:85). Yield: 15.5 mg(3%. A, C, D, L and E)
9. DO3A10CM-Dna12-Arg-Trp-Ser-His-Darg-Trp-Bpa4-Pro-Ae (BRU-2981) (SEQ ID NO: 24). Yield: 56.0 mg(36.6%; A, C, D, G, L and E)
10. DO3A10CM-Dna12-Arg-Trp-Ser-His-Darg-Trp-Bpa4-Pro-Aprp1h3 (BRU-2982) (SEQ ID NO: 24). Yield: 12.3 mg (7.9%; A, C, D, G, L and E)
11. DO3A10CM-Dna12-Arg-Trp-Ser-His-Darg-Trp-Bpa4-Pro-Pheol (BRU-2983). Yield: 25.4 mg (5.1%; A, C, D, G, L and E)
12. DO3A10CM-Dna12-Arg-Trp-Ser-His-Darg-Trp-Bpa4-Pro-Gua (BRU-2984). Yield: 16.0 mg( 10.4%; A, C, D, G, L and E; guanidine carbonate was used as the amine equiv.)
13. DO3A10CM-Dna12-Arg-Trp-Ser-His-Darg-Trp-Bpa4-Pro-A1guao3pt (BRU-2985). Yield: 15.5 mg (9.6%; A, C, D, F, I, L and E)
14. DO3A10CM-Dna12-Arg-Trp-Ser-His-Darg-Trp-Bpa4-Pro-Gly-Aprp1h3 (BRU-2987) (SEQ ID NO: 84). Yield: 30.0 mg (18.6%; A, C, D, G, L and E)
15. DO3A10CM-Dna12-Arg-Trp-Ser-His-Darg-Trp-Bpa4-Pro-Gly-Abt1h4 (BRU-2986) (SEQ ID NO: 84). Yield: 11.3 mg (7%; A, C, D, G, L and E)
16. DO3A10CM-Dna12-Arg-Trp-Ser-His-Darg-Trp-Bpa-Pro-Gly-Abn (BRU-2988) (SEQ ID NO: 84). Yield: 18.2 mg (11 %; A, C, D, G, L and E)
17. DO3A10CM-Dna12-Arg-Trp-Ser-His-Darg-Trp-Bpa4-Pro-Gly-Az23(py2)2po-Ap (BRU- 2989) (SEQ ID NO: 84). Yield: 1.2 mg (0.7%; A, C, D, J, L and E)
18. DO3A10CM-Dna12-Arg-Trp-Ser-His-Darg-Trp-Bpa4-Pro-Gly-NH₂ (BRU-3005) (SEQ ID NO: 85). Yield: 12.7 mg (2.7%; A, C, D, L and E)
19. DO3A10CM-Dna12-Arg-Trp-Ser-His-Darg-Trp-Bpa4-Pro-Ap (BRU-3006). Yield: 5.6 mg (1.2%; A, C, D, G, L and E)
20. Sar-Arg-Trp-Ser-His-Darg-Trp-Bpa4-Pro-Da15o3pt (BRU-3007). Yield: 59.0 mg (16.4%; A, B, C, D, F and E)
21. DO3A10CM-Sar-Arg-Trp-Ser-His-Darg-Trp-Bpa4-Pro-Az23po-Dabt14 (BRU-3019) (SEQ ID NO: 8). Yield: 20.5 mg(4.4%; A, C, D, H, Land E)
22. DO3A10CM-Dna12-Arg-Trp-Ser-His-Darg-Trp-Bpa4-Pro-Mo2abn (BRU-3020). Yield: 26.5 mg (16.6%; A, C, D, G, L and E)
23. DO3A10CM-Dna12-Arg-Trp-Ser-His-Darg-Trp-Bpa4-Pro-Az23m2po-NH₂ (BRU-3021), Yield: 18.7 mg(3.9%; A, C, D, H, I and E)
24. DO3A10CM-Sar-Arg-Trp-Ser-His-Darg-Trp-Bpa4-Pro-Az23po-Da15o3pt (BRU-3022) (SEQ ID NO: 8). Yield: 31.0 mg (6.6%; A, C, D, H, L and E)
25. DO3A10CM-Dna12-Arg-Trp-Ser-His-Darg-Trp-OH (BRU-3046) (SEQ ID NO: 86). Yield: 53.5 mg (14.2%; A, C, D, L and E)
26. DO3A10CM-Dna12-Arg-Trp-Ser-His-Darg-Trp-Bpa4-OH (BRU-3064) (SEQ ID NO: 87), Yield: 0.119 g (27%; A, C, D, K, L and E)

### Protocol-A: Solid Phase Peptide Synthesis

Fully protected Fmoc-Dnal2/Fmoc-Sar-Arg(Pmc)-Trp(Boc)-Ser(Bu)-His(Trt)-Darg(Pmc/Pbf)-Trp(Boc)-Bpa4-Pro-OH and Fmoc-Dnal2(Fmoc-Sar)-Arg(Pmc)-Trp(Boc)-Ser(Bu)-His(Trt)-Darg(Pmc/Pbf)-Trp(Boc)-Bpa4-Pro-Gly-OH were prepared on either Fmoc-Pro-NovaSyn-TGT® resin (0.22 mmol/g) and/or Fmoc-Gly-NovaSyn-TGT® resin (0.22 mmol/g) using an ABI 433A instrument (Applied Biosystems, Foster City, Calif.). The peptides were assembled on resin using the FastMoc™ protocol, usually on a 0.25 mmol scale. After chain elongation was completed, the resin was washed with DCM (4×). The resin was then transferred to a manual peptide synthesizer vessel and shaken with 70:30 dichloromethane/hexafluoroisopropanol for 1 h. The resin was drained and washed with 2×10 mL of dichloromethane. The combined filtrates were concentrated under reduced pressure to yield the fully protected peptide sequence with a free carboxylic acid group at the C-terminus as colorless foam.

When a C-terminus free acid was not required, the entire peptide chain was built on Fmoc-PAL-PEG-PS resin (0.22 mmol/g, 1.14 g, 0.25 mmol) on an ABI 433A automated peptide synthesizer using FastMoc™ protocols, except for the coupling of DO3A10CM(tris-t-butyl), which was carried out manually in a peptide synthesis vessel (Refer to protocol L).

Protocol-B: Manual Coupling of Amino Acids with HBTU

To the required amino acid (4 equiv) in dry DMF (3.3-4.5 mL/mmol) was added successively HBTU (4 equiv), HOBt.H₂O (4 equiv) and DIEA (8.8 equiv) and the vessel was agitated for 10 min at ambient temperature. The solution of the activated acid was then transferred to the free amino group-bearing resin, suspended in DMF, and the reaction vessel was shaken for 6 h at ambient temperature. The resin was drained, washed with DMF (50 mL/mmol) after which further chain elongation or elaboration was conducted.

### Protocol-C: Manual Removal of the Fmoc Protecting Group

The resin containing the Fmoc-protected amino acid was treated with 20% piperidine in DMF (v/v, 15 mL/g resin) for 10 min. The solution was drained from the resin. This procedure was repeated once more followed by washing the resin with DMF (4×).

Protocol-D: Manual Deprotection of the Peptides from the Resin/Solution Phase Synthesis

20.0 mL of Reagent A (95:2.5:2.5—TFA:Water:TIPS) was added to the resin in a manual peptide synthesizer vessel or to the final crude peptide prepared by solution phase in a RB flask and was shaken/stirred for 4 h at ambient temperature. The resin was filtered, washed with 3×5 mL of TFA and combined with the filtrate. The solutions from both solid phase and solution phase were then concentrated to a paste under reduced pressure at RT and the crude peptide was precipitated with 20 mL of absolute ether. The precipitate was washed with 2×10 mL of dry ether and then purified by preparative HPLC.

### Protocol-E: Purification of the Crude Peptides by Preparative HPLC

The crude peptides were dissolved in approximately 10 mL of distilled, deionized water. Where required, ACN was added dropwise until the solution became homogeneous (the amount of ACN did not exceed more than 20%-v/v). The solution was filtered through a 25.0μ. PTFE filter, loaded onto a preparative column using a ternary pump and purified by preparative HPLC.

Column: Atlantis-C₁₈, RP; Particle size: 10.0μ; Solvent A: H₂O with 0.1% TFA; Solvent B: ACN with 0.1% TFA; Elution rate: 100.0 mL/min; Detection @ 220 nm; Initial conditions: 10% B; Gradient: 10-20% B over 10.0 min and 20-70% B over 100 min. Fractions with the required mass and >95% purity were pooled and freeze dried to yield the peptide as a TFA salt.

### Protocol-F: Synthesis of Peptide Sequences on the Diamine-Loaded Trityl Resin

The first amino acid was activated as detailed in protocol B and transferred to the diamine-loaded trityl resin (0.25 mmol) in a manual peptide synthesizer vessel followed by agitation for 12 h. The resin was drained and washed with DMF (3×15 mL). The resin was then transferred to a peptide vessel on the ABI peptide synthesizer and the rest of the sequence was added using ABI FastMoc™ protocols. The final coupling of DO3A10CM(tris-t-butyl) was carried out manually as detailed in protocol L.

### Protocol-G: Synthesis of Peptides Bearing C-Terminus Aminoalkyl Groups/Aminohydroxy-Alkyl Groups

About 0.081 mmol (about 0.2 g from procedure A) of the fully protected DO3A10CM-tris-t-butyl ester-bearing peptide sequence with a free carboxyl at the C-terminus was dissolved in 200 μL of DMF. This was treated sequentially with 0.81 mmol of N-hydroxysuccinimide and 1.0 mmol of DIC followed by stirring at ambient temp for 4 h. The resulting crude NHS ester was then added dropwise to a solution of the requisite alkylamine/hydroxyalkylamine (2.0 mmol) in 200 μL of DMF over a period of 10 min with vigorous stirring. After nearly 16 h, the reaction mixture was diluted with 100 mL of water and the aqueous solution was extracted with 3×50 mL of EtOAc. The combined organic layers were washed with water (2×50 mL), saturated sodium carbonate (2×50 mL), water (2×50 mL) and finally with saturated NaCl solution (1×50 mL) and dried (Na₂SO₄). The solution was filtered from the drying agent, concentrated under reduced pressure to a paste, and the crude peptide was dried in vacuo for 1 h. This material was then deblocked using Reagent A and purified by preparative HPLC.

Protocol-H: Synthesis of Modified azaGly on the Resin

Diamine-bearing trityl resin and/or free-amine-bearing PAL-PEG-PS resin (Fmoc already removed) (0.25 mmol) was suspended in 10 mL of anhydrous THF. CDI (2.5 mmol) was added, followed by shaking in a manual peptide synthesis vessel for 4 h. The resin was drained and washed with a 1% solution of the required hydrazine in DMF (3×20 mL). The resin was again washed with DMF (3×20 mL) and agitated with 20 mmol of the required hydrazine in 20 mL of DMF for 12 h. The resin was drained, washed with DMF (3×20 mL) and submitted to the next coupling.

The required amino acid (1.0 mmol) was dissolved in 10 mL of anhydrous THF and cooled to −10° C. and kept under nitrogen atmosphere. Isobutyl chloroformate (1.0 mmol) was added to the amino acid via syringe with stirring followed by NMM (1.01 mmol) in THF. The reaction mixture was allowed to come to 0° C. and stirred for 30 min. This activated acid was then transferred to the mixed urea on the resin and agitated for 12 h. The resin was then drained and washed with 3×20 mL of 1:1 DMF/MeOH and then with 3×20 mL of DMF. The resulting peptide segment on the resin was taken through the rest of the chain elongation on an ABI automated peptide synthesizer.

### Protocol-I: Solution-Phase Guanylation of Amines

The completed peptide chain on diamine bearing trityl resin was cleaved from the resin using 95:5:0.1%—DCM:TFA:TIPS (1 h) and the filtrate was concentrated under reduced pressure to a paste. The paste was dried in vacuo and then redissolved in 5.0 mL of acetonitrile. To this solution, 2.0 mmol of triethylamine was added, followed by 1.0 mmol of solid N,N′-di-Boc-S-methylisothiourea. The reaction mixture was stirred at ambient temp for 20 h. Volatiles were removed under reduced pressure and the protecting groups on the peptide were removed with Reagent A for 4 h. Volatiles were removed under reduced pressure and the residue was purified preparative HPLC (Refer to protocol E).

Protocol-J: Introduction of Substituted azaGly by Solution-Phase Synthesis

To a solution of 0.2 g of DO3A10CM(tris-t-butyl ester)-Dnal2-R(Pmc)-W(Boc)-S(Bu)-H(Trt)-Darg(Pmc)-W(Boc)-Bpa4-P-G-OH (0.08 mmol) in dry THF (0.5 mL) cooled to −10° C., NMM (0.088 mmol) and isobutylchloroformate (0.08 mmol) were added successively. The solution was allowed to come to 0° C. and stirred for 30 min. N-Amino(phenylamino)-N-(2-pyridyl)carboxamide⁴⁸ (0.17 mmol) was added and stirring was continued for 20 h more. Volatiles were removed under reduced pressure and the residue was deprotected and purified by preparative HPLC as described in protocols D and E.

Protocol-K: Loading of the First Amino Acid onto 2-Chlorotrityl Chloride Resin

2-Chlorotrityl chloride resin (0.25 mmol) was pre-swelled for 15 min with 1:1—DMF:DCM in a peptide synthesis vessel after which the solvent mixture was drained. A solution of 1.0 mmol of the first Fmoc-amino acid and 2.2 mmol of DIEA in DMF (5.0 mL) was added to the vessel followed by agitation for 12 h. The vessel was drained by application of positive nitrogen pressure. MeOH (10 mmol) in DMF (15 mL) containing DIEA (10 mmol) was added to the vessel and the vessel was shaken for 1 h. The vessel was drained and washed with MeOH (3×15 mL) and DMF (4×15 mL). The resulting resin was ready for chain elongation on the ABI 433A instrument and further addition of DO3A10CM(tris-t-butyl) manually (the loading was assumed to be 100%).

Protocol-L: General Procedure for Introduction of DO3A10CM onto the Resin

DO3A10CM-tris-t-butyl ester (4.0 eq.), HOBt.H₂O (4.0 eq) and HBTU (4.0 eq) were dissolved in 5.0 mL of DMF and DIEA (8.8 eq) was added followed by stirring at RT for 10 min. Thr resulting solution of the activated acid in DMF was transferred to the amine-bearing resin in a peptide synthesis vessel and an additional 1.0 mL of DMF was used to transfer the remaining activated acid to the amine. The total volume of the suspension was brought to about 10 mL with DMF and the vessel was agitated for 20 h at ambient temperature. The vessel was drained and washed with 3×15 mL of DMF and 3×15 mL of DCM. Then the peptide was cleaved, deprotected and purified using protocols D and E.

Synthesis of LHRH-II Analogs with DO3A10CM on the N-terminus and azaGly at Position 10

All linear peptides were synthesized on a 50-1.μmol scale using Fmoc chemistry and PAL-PEG-PS resin (0.2 mmol/g) using an established automated protocol on a Symphony® Peptide Synthesizer (twelve peptide sequences/synthesis). Coupling of amino acids was performed for 1 h with a 4-fold excess each of amino acid and PyBOP/DIEA in DMF.

To synthesize analogs of LHRH-II peptides with an azaGly¹⁰ moiety, azaGly-loaded resin was prepared using the versatile and very convenient method⁴⁹ involving the appendage of a reactive carboimidazole group to the resin-bound amino group using N,N-carbonyldiimidazole followed by displacement of imidazole from the carboimidazole intermediate with hydrazine (Scheme 1). Thus, resin-Fmoc-PAL-PEG-PS was treated with 20% piperidine in DMF and followed by N,N-carbonyldiimidazole (10 eq.) in DMF for 5 h. The reactive carboimidazole intermediate was reacted with hydrazine (4 eq.) to provide the azaGly moiety on the resin. Since the stability of the resin loaded with azaGly on storage unknown, it was coupled with the amino acid destined for position 9, namely, Fmoc-Pro-OH using PyBOP/DIEA. After loading Fmoc-Pro-azaGly-, the resin could be stored at 0-4° C. without degradation and the Fmoc-Pro-azaGly-PAL-PEG-PS resin (substitution level 0.2 mmol/g) was used for synthesis. This method of preparation of peptides with azaGly at the C-terminus was found to be superior to the method involving the conversion of the C-terminal hydrazide moiety using sodium cyanate/acetic acid^{50,51} or the method involving the laborious azide coupling with semicarbazide.^{50,52}

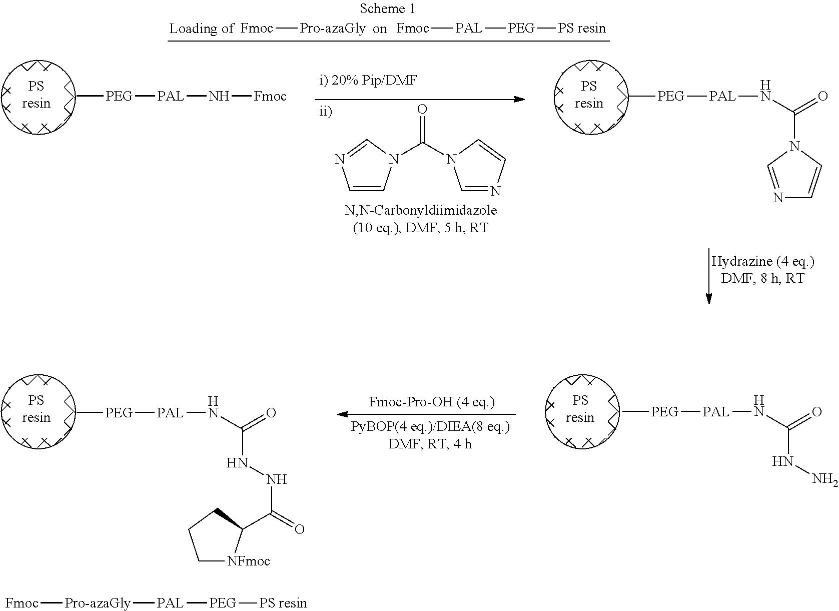

To introduce the N-substituted glycine derivative⁴³ at position AA¹ during solid phase synthesis, bromoacetic acid was loaded instead of Gly, using DIC as the coupling agent followed by displacement of bromide by the requisite primary amine as appropriate.

In a typical procedure (as represented by BRU-2440 (seq004), Scheme 2), the peptide AA¹-AA⁹/AA¹⁰ was prepared using solid phase synthesis on an automated synthesizer (Rainin Symphony® Peptide Synthesizer, twelve peptide sequences/synthesis) and the fully protected peptide was treated with 20% Pip/DMF to remove the Fmoc-group from the resin to furnish the chain with a free amine at the N-terminus. After chain assembly of the desired LHRH-II sequence, the protected chelating group DO3A10CM-tris-t-butyl ester (6 eq.) was coupled to the N-terminal amino acid using PyBOP/DIEA for 18 h to ensure the complete loading of the chelator. Since the attachment of the chelating agent could not be achieved on the N-terminus of the natural LHRH-II sequence, which contains pyroglutamic acid (pGlu) at position 1, pGlu was replaced by sarcosine. It was reasoned that replacement of pGlu¹ by Sar or by a D-amino acid (e.g., Dnal2) would decrease the rate of degradation of the peptide by pyroglutamate aminopeptidase.⁵³ After completion of the peptide synthesis, the resin was subjected to an automated ‘on-board’ cleavage protocol with the cleavage cocktail, “Reagent B” (TFA:water:phenol:triisopropylsilane, 88:5:5:2, v/v/w/v) (10 mL/g of resin) for 4 h. Isolated crude peptides were purified by reversed phase HPLC chromatography on a C18 column (Waters, XTerra® Prep MS C18, 10μ, 300 Å, 50×250 mm) using water (0.1% TFA) and CH₃CN (0.1% TFA, v/v) as eluents. Peptides isolated after purification were analyzed using analytical HPLC and mass spectroscopy to confirm the purity. Those of >95% purity were employed in EFO-27 cell-binding studies.

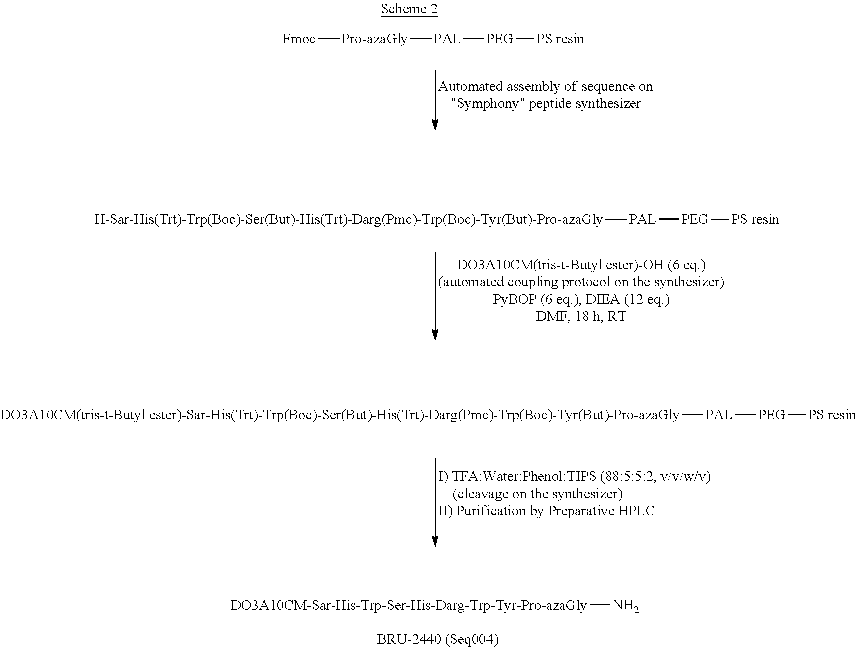

Synthesis of LHRH-II Analogs Bearing DO3A10CM at the N-terminus and Functionalized Amines at position 10

At the outset, LHRH-II analogs with various alkylamines at position 10 seemed amenable to synthesis by peptide synthesis methods. However during the course of the work it became clear that standard peptide synthesis protocols could not be used for all the steps needed to complete the synthesis. Solid phase and solution phase synthetic techniques and/or improvements to the existing synthetic protocols were required. These peptides were prepared by three methods.

In method 1, amino acid chain AA¹-AA⁹/AA¹⁰ was prepared using solid phase synthesis on an automated synthesizer (ABI, Applied Biosystems, Inc.). The peptide was then cleaved from the resin and deprotected by ‘Reagent B’ to furnish the chain with a free carboxylic acid at the C-terminus

In method 2, amidation of the fully protected peptide with an activated acid at the C-terminus acid (NHS/DIC) using excess diamine in solution resulted in a free amino group at the C-terminus as the major component. Our initial attempts to prepare these peptides on solid phase starting from trityl resins that were loaded with diamines either failed or resulted in a mixture of products and the isolation of the required products in high purity proved very cumbersome.

A third method involved the construction of a substituted semicarbazide on the resin. Attempted preparation of the required semicarbazide was started from the corresponding diamine-bearing trityl resin. The amine on the resin was sequentially treated with CDI followed by hydrazine to assemble the semicarbazide. However attempted acylation of this with the first amino acid using known coupling agents and conditions (PyBOP, HBTU, HATU etc) failed. To overcome this difficulty, the amino acid was activated with isobutylchloroformate and NMM to form the mixed anhydride, which was added to the semicarbazide. The acylation was carried out for 12 h. The reaction proceeded as expected and the rest of the peptide chain was then built on the resin with the aid of an automated synthesizer. After the final amino acid was added, the peptide was cleaved from the resin and acylated with DO3A-tris-t-butyl ester, deprotected and purified to yield the required LHRH-II analog. Isolated peptides after HPLC purification were analyzed by HPLC and mass spectrometry to confirm their purity.

Development of Potent LHRH-II Analogs with a Chelator at the N-terminus

The synthetic efforts were largely devoted to development of peptides with increased binding affinity to the presumed LHRH receptor in EFO-27 cells and resistance to degradation or first-pass excretion, characteristics that, for the LHRH analogs, are generally interrelated.^{54,55} To study the effect of replacement of amino acids in different positions in the sequence of metal-chelate-bearing LHRH analogs on binding and biological activity, many LHRH-II analogs, including those shown in Table 24, were synthesized. A proposed type II′ β-turn conformation for BRU-2813 is shown below. This compound, which bears a multidentate chelator known as DO3A10CM on its N-terminus, is a representative example of the compounds prepared.

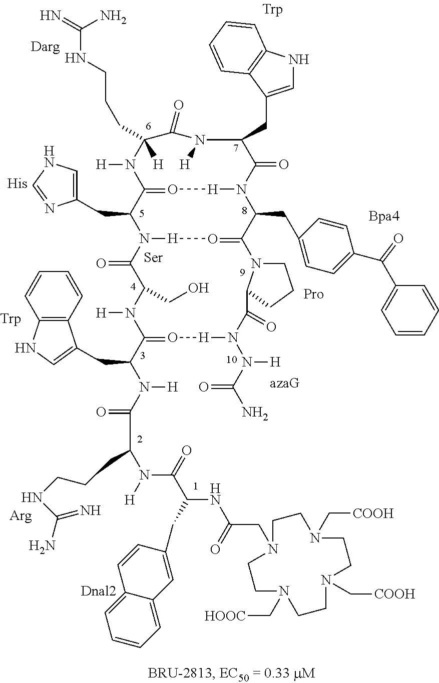

### Schematic Representation of a Typical LHRH-Targeted Compound Synthesized

This analog, developed in the early part of our effort was identified to have better binding potency than the radio-iodinated LHRH standard, [Darg⁶, ¹²⁵I-Tyr⁸, azaGly¹⁰-LHRH-II] ([¹²⁵I-Tyr]BRU-2477) for binding to cancer cells. All compounds prepared were tested for specific binding to EFO-27 cells; their abilities to compete for binding to cancer cells in a standard cell-based plate-assay relative to [Darg⁶, ¹²⁵I-Tyr⁸,azaGly¹⁰-LHRH-II] ([¹²⁵I-Tyr⁸]BRU-2477) (EC₅₀ data) were determined and structure-function analysis was performed for all compounds using the assay methods described below.

### Cell Culture

The EFO27 human ovarian cancer cells were obtained from the American Type Culture Collection and cultured in growth medium, RPMI 1640 (Cellgro) supplemented with 10% fetal bovine serum. The cultures were maintained in a humidified atmosphere containing 5% CO₂/95% air at 37° C. and passaged and harvested routinely using 0.05% trypsin/EDTA.

### Competition Binding Assay

LHRH compounds were screened in a standard cell-based plate assay for their ability to compete with the radio-iodinated LHRH, [Darg⁶, ¹²⁵I-Tyr⁸,azaGly¹⁰-LHRH-II] ([¹²⁵I-Tyr⁸]BRU-2477) for binding to cancer cells. BRU-2477 is the principal LHRH-II analog disclosed in the Siler-Khodr patent referred to earlier herein. Briefly, EFO-27 cells were cultured and seeded in 96-well clear flat bottom plates at 30,000/well density in growth medium and were used for the assay at 100% confluence the following day, after a wash with chilled phosphate-buffered saline pH 7.4 (PBS). The binding assay was carried out by incubating cells with [¹²⁵I-Tyr⁸]BRU-2477 in the absence or presence of varying concentrations of test compounds for 90 min at −10° C. All compounds were diluted in phosphate buffered saline (VWR CAT#45000-434) supplemented with 20 mM HEPES, 0.1% BSA, 0.5 mM PMSF (AEBSF), bacitracin (100 mg/L), pH 7.4. At the end of incubation, cells were washed with PBS and the radioactivity associated with each well was read using a Microplate Scintillation counter. [¹²⁵I-Tyr⁸]BRU-2477 was custom made by GE Healthcare (Woburn, Mass.) and supplied as freeze-dried powder with a radiochemical purity (RCP) of >95% and specific activity of 2000 Ci/mmol. The competition binding data were analyzed by Graphpad Prizm™ software to determine EC₅₀ values, the effective concentration of test compound that inhibits [¹²⁵I-Tyr⁸]BRU-2477 binding by 50%. These data are provided in various tables presented throughout this specification.

### Direct Binding Assay

All reagents and chemicals were obtained from Sigma unless otherwise specified. LHRH analogs and ¹⁷⁷Lu-LHRH-II analogs were prepared by in-house chemists, as described elsewhere in the application. ¹⁷⁷Lu-LHRH-II analogs were not HPLC purified, as they had been prepared using formulation conditions that yielded high RCP without the need for purification. Radiolabeled products had an average specific activity of 1.1 Ci/umole and their radiochemical purity ranged from 75-90%. ¹²⁵I-LHRH II (IMQ7611v) ([¹²⁵I-Tyr⁸]BRU-2477) was custom labeled by GE-Healthcare using the lactoperoxidase method with a specific activity of 2000 Ci/mmole and >99% RCP. The HPLC-purified material was taken in a stabilizing buffer containing 5% lactose, 0.1% L-cysteine hydrochloride and 800 KIU/mL aprotinin and received as a lyophilized product and stored at −70° C. This was reconstituted in distilled water, aliquoted and stored at −70° C. The radioactivity was determined using Microplate Scintillation counter (Wallac Microbeta Trilux).

Cell Culture: EFO-27 human ovarian cancer cells were obtained from the American Type Culture Collection and cultured in the growth medium, RPMI 1640 (Cellgro) supplemented with 10% fetal bovine serum(Hyclone, SH30070.03). The cultures were maintained in a humidified atmosphere containing 5% CO₂/95% air at 37° C. and passaged routinely using 0.25% trypsin/EDTA. For the binding assay, EFO-27 cells were seeded onto 96-well clear flat-bottom tissue-culture-treated plates at 15,000/well density in the growth medium and used for assay on day 2 post-seeding. Cells were routinely checked for confluence and contamination and cell count was done occasionally to ensure consistency in cell numbers.

Direct Binding: Direct binding studies were carried out by incubating appropriate ¹⁷⁷Lu-labeled compounds with EFO-27 cells at 4° C. for 1 h followed by washing off the unbound radioactivity. Non-specific binding was determined by incubating the ¹⁷⁷Lu-LHRH-II analogs in the presence of a large excess (30 uM) of cold (unlabeled) LHRH-II analogs. A 96-well plate format was used.

Internalization/Efflux Studies: The internalization and efflux studies were carried out following the general procedure. Basically, ¹⁷⁷Lu-LHRH II or ¹²⁵I-LHRH II ([¹²⁵I-Tyr⁸]BRU-2477) (75 uL, 3.0 μCi/mL) was added to the EFO-27 or PC-3 cells. The cells were incubated for 40 min at 37° C. The unbound radioactivity was washed off (4×). Following addition of fresh medium, the cells were further incubated for up to 2 h. At various time points (15, 30, 60 & 120 min) the distribution of radioactivity (membrane-bound, internalized, and efflux) was determined

The results obtained using these assay methods were used to develop the structure activity relationships (SAR) described below.

### Initial Studies: Chelator Attachment

The initial synthetic starting point, an LHRH-II agonist known⁴⁵ to have good biological activity and enhanced stability, namely pGlu-His-Trp-Ser-His-Darg-Trp-Tyr-Pro-azaGly-NH₂ (BRU-2477), does not contain any potential point of attachment for a metal chelator such as DO3A10CM. Its Dlys⁶ analog (BRU-2437) was prepared, and found to have a significantly poorer EC₅₀ (8.54 vs 0.74 μM for the Dlys⁶ and Darg⁶ analogs respectively in binding studies; in competition with the radio-iodinated LHRH, [Darg⁶, ¹²⁵I-Tyr8,azaGly10-LHRH-II] (¹[¹²⁵I-Tyr⁸]BRU-2477) on EFO-27 cells. Attachment of DO3A10CM to the Dlys⁶ was attempted, based on literature indicating that such substitution was well tolerated for LHRH-I analogs, but this compound was also a weak binder. DO3A10CM may be too sterically demanding at this position, so modification of the pyroglutamic acid to allow N-terminus attachment of the chelate was evaluated. Returning to peptides with the Darg⁶ substituent, pGlu was replaced with sarcosine (N-methylglycine). Surprisingly, this modification of the N-terminus was tolerated, though subsequent attachment of an N-terminus DO3A10CM was not, yielding an EC₅₀ of >10 μM. Also surprisingly, replacement of Tyr⁸ with Bpa4 improved the EC₅₀ ten-fold, suggesting that a further study of modifications at position 8 was warranted. The sequences and cell binding results obtained with these constructs are shown in Table 2.

TABLE 2
Preliminary Compounds

| | Chelating | | | | | | | | | | | | |
|---|---|---|---|---|---|---|---|---|---|---|---|---|---|
| Seq. # | Group | AA¹ | AA² | AA³ | AA⁴ | AA⁵ | AA⁶ | AA⁷ | AA⁸ | AA⁹ | AA¹⁰ | BRU No. | EC₅₀ μM |
| Seq009 | — | pGlu | H | W | S | H | Darg | W | Tyr | P | azaG | BRU-2477 | 0.74 |
| Seq001 | — | pGlu | H | W | S | H | Dlys | W | Tyr | P | azaG | BRU-2437 | 8.54 |
| Seq003 | — | Sar | H | W | S | H | Darg | W | Tyr | P | azaG | BRU-2439 | 0.25 |
| Seq054 | DO3A10CM | Sar | H | W | S | H | Darg | W | Tyr | P | azaG | BRU-2758 | 10.00 |
| Seq007 | DO3A10CM | Sar | H | W | S | H | Darg | W | Bpa4 | P | azaG | BRU-2443 | 0.95 |

BRU Nos. 2477, 2437, 2758 and 2443 in Table 2 immediately above correspond, respectively, to SEQ ID NOs: 88, 89, 14 and 12 in the Sequence Listing.

### Modifications at Position 8: Effect of Lipophilicity

The initial binding studies of [Sar¹, Darg⁶, azaGly¹⁰]LHRH-II (BRU-2439) on human ovarian cancer (EFO-27 cells) showed a significant in vitro binding potency (EC₅₀=0.25 μM); the binding effect presumably was influenced by the known potency enhancing effect of the D-amino acid (Darg) at the 6 position and an azaglycine amide at position 10. Replacement of Tyr⁸ by more hydrophobic L-4-benzoylphenylalanine (Bpa4) provided BRU-2441 ([Bpa4⁸]BRU-2439, EC₅₀=0.14 μM) which was ˜2 times more potent than BRU-2439 (EC₅₀=0.25 μM). This increase in binding led to synthesis of a series of LHRH-II analogs that contained both DO3A10CM on the N-terminus and modifications at position 8 using amino acids with varied lipophilicity and in some cases bearing basic groups. The cell binding results obtained with these constructs are shown in Table 3.

TABLE 3
LHRH Peptides with Modification of the Amino Acid at Position 8

| | | | | | | | | | | | | | | EC₅₀ |
|---|---|---|---|---|---|---|---|---|---|---|---|---|---|---|
| No | Seq. # | Chelating Group | AA¹ | AA² | AA³ | AA⁴ | AA⁵ | AA⁶ | AA⁷ | AA⁸ | AA⁹ | AA¹⁰ | BRU No. | μM |
| 1 | Seq003 | — | Sar | H | W | S | H | r | W | Tyr | P | azaG | BRU-2439 | 0.25 |
| 2 | Seq005 | — | Sar | H | W | S | H | r | W | Bpa4 | P | azaG | BRU-2441 | 0.14 |
| 3 | Seq007 | DO3A10CM | Sar | H | W | S | H | r | W | Bpa4 | P | azaG | BRU-2443 | 0.95 |
| 4 | Seq010 | DO3A10CM | Sar | H | W | S | H | r | W | Nal2 | P | azaG | BRU-2624 | 2.01 |
| 5 | Seq011 | DO3A10CM | Sar | H | W | S | H | r | W | Bip | P | azaG | BRU-2625 | 2.14 |
| 6 | Seq012 | DO3A10CM | Sar | H | W | S | H | r | W | Tbufe4 | P | azaG | BRU-2634 | 0.98 |
| 7 | Seq022 | DO3A10CM | Sar | H | W | S | H | r | W | Lys(isp) | P | azaG | BRU-2675 | 12.34 |
| 8 | Seq027 | DO3A10CM | Sar | H | W | S | H | r | W | Cha | P | azaG | BRU-2718 | 0.63 |
| 9 | Seq029 | DO3A10CM | Sar | H | W | S | H | r | W | Nal1 | P | azaG | BRU-2720 | 0.54 |
| 10 | Seq030 | DO3A10CM | Sar | H | W | S | H | r | W | Dip | P | azaG | BRU-2721 | 4.55 |
| 11 | Seq031 | DO3A10CM | Sar | H | W | S | H | r | W | F₅fe | P | azaG | BRU-2722 | 4.62 |
| 12 | Seq032 | DO3A10CM | Sar | H | W | S | H | r | W | Arg | P | azaG | BRU-2723 | 2.83 |
| 13 | Seq033 | DO3A10CM | Sar | H | W | S | H | r | W | Trp | P | azaG | BRU-2724 | 2.67 |
| 14 | Seq034 | DO3A10CM | Sar | H | W | S | H | r | W | Cfe4 | P | azaG | BRU-2725 | 2.26 |
| 15 | Seq035 | DO3A10CM | Sar | H | W | S | H | r | W | Pal3 | P | azaG | BRU-2726 | 17.40 |
| 16 | Seq039 | DO3A10CM | Sar | H | W | S | H | r | W | Phe | P | azaG | BRU-2730 | 12.89 |
| 17 | Seq050 | DO3A10CM | Sar | H | W | S | H | r | W | Tha | P | azaG | BRU-2742 | 10.00 |
| 18 | Seq054 | DO3A10CM | Sar | H | W | S | H | r | W | Tyr | P | azaG | BRU-2758 | 10.00 |
| 19 | Seq056 | DO3A10CM | Sar | H | W | S | H | r | W | Amfe4 | P | azaG | BRU-2760 | 3.55 |
| 20 | Seq059 | DO3A10CM | Sar | H | W | S | H | r | W | His | P | azaG | BRU-2763 | 13.55 |
| 21 | Seq060 | DO3A10CM | Sar | H | W | S | H | r | W | Aic2 | P | azaG | BRU-2764 | 4.31 |
| 22 | Seq061 | DO3A10CM | Sar | H | W | S | H | r | W | Ing2 | P | azaG | BRU-2765 | 8.71 |
| 23 | Seq062 | DO3A10CM | Sar | H | W | S | H | r | W | — | P | azaG | BRU-2767 | 10.00 |
| 24 | Seq090 | DO3A10CM | Sar | H | W | S | H | r | W | Dtyr(OBz) | P | azaG | BRU-2819 | 0.77 |
| 25 | Seq091 | DO3A10CM | Sar | H | W | S | H | r | W | Tyr(OBz) | P | azaG | BRU-2820 | 1.57 |
| 26 | Seq094 | DO3A10CM | Sar | H | W | S | H | r | W | Thy | P | azaG | BRU-2823 | 1.61 |
| 27 | Seq113 | DO3A10CM | Sar | H | W | S | H | r | W | Bpa(NOH) | P | azaG | BRU-2907 | 1.90 |

BRU Nos. 2624, 2625, 2634, 2675, 2718, 2720, 2721, 2722, 2723, 2724, 2725, 2726, 2730, 2742, 2758, 2760, 2763, 2764, 2765, 2767, 2819, 2820, 2823 and 2907 in Table 3 immediately above correspond, respectively, to SEQ ID NOs: 90, 91, 92, 93, 94, 95, 96, 97, 98, 99, 100, 101, 102, 103, 104, 105, 106, 107, 108, 109, 110, 111, 112 and 113 in the Sequence Listing.

The binding data in Table 3 indicated that the lipophilicity of the amino acid at position 8 played an important role in influencing the in vitro potency. At this position, amino acids with basic character tended to reduce the binding efficiency.

A very interesting result was drawn (Table 4) by comparing the binding potencies of analogs containing amino acids, Bpa4 (BRU-2443, EC₅₀=0.95 μM), Nal2 (BRU-2624, EC₅₀)=2.01 μM), Bip (BRU-2625, EC₅₀=2.14 μM), Nal1 (BRU-2720, EC₅₀=0.54 μM), Dip (BRU-2721, EC₅₀=4.55 μM), Trp (BRU-2724, EC₅₀=2.67 μM), Ing2 (BRU-2765, EC₅₀=8.71 μM) and Thy (BRU-2823, EC₅₀=1.61 μM). These data indicate that an amino acid with a linear aromatic hydrophobic moiety increased the binding. Binding was also influenced positively by the presence of a group like C=0 (Bpa4, BRU-2443).

TABLE 4
Comparison of EC₅₀ Values of Some LHRH-II Analogs with Selected Hydrophobic AA⁸

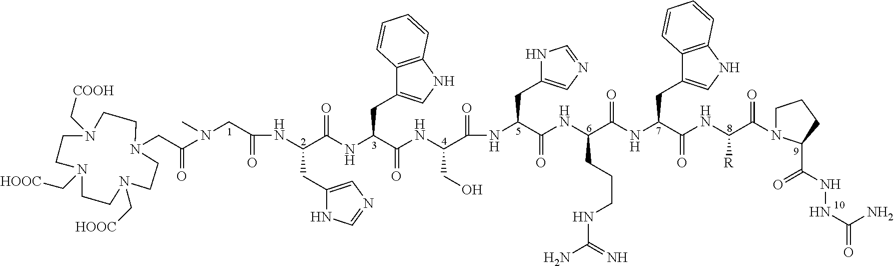

| | | | | EC₅₀ |
|---|---|---|---|---|
| Seq. # | BRU No. | AA⁸ | R | (μM) |
| Seq007 | BRU-2443 | L-4-Benzoylphenylalanine Bpa4 | 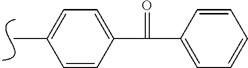 | 0.95 |
| Seq010 | BRU-2624 | L-2-Naphthylalanine Nal2 | 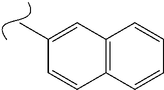 | 2.01 |
| Seq011 | BRU-2625 | L-Biphenylalanine Bip | 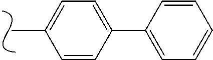 | 2.14 |
| Seq029 | BRU-2720 | L-1-Naphthylalanine Nal1 | 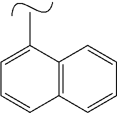 | 0.54 |
| Seq030 | BRU-2721 | L-Diphenylalanine Dip | 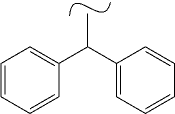 | 4.55 |
| Seq033 | BRU-2724 | L-Tryptophan Trp | 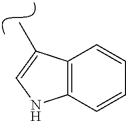 | 2.67 |
| Seq061 | BRU-2765 | 2-Indanyl-L-glycine Ing2 | 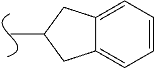 | 8.71 |
| Seq094 | BRU-2823 | L-Thyronine Thy | 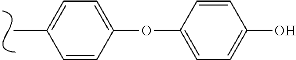 | 1.61 |

Since the incorporation of Bpa4 at position 8 provided an LHRH-II analog with enhanced potency, [Bpa4⁸]LHRH-II (BRU-2443) was considered thereafter as the starting sequence for structure-function relationship studies. In subsequent synthesis of other LHRH-II analogs with modifications at various positions in the sequence, Bpa4 at position 8 was (in general) kept constant.

### Modifications at Positions 1 and 2: Efforts to Increase Hydrophobicity

In an initial attempt to explore the effect of hydrophobicity at position 1 of LHRH-II analogs, a peptide with Dnal2 (2-naphthyl-D-alanine), BRU-2666 (EC₅₀=2.60 μM) was synthesized and it was found to be 2 times more potent than the similarly constituted LHRH-II analog with Sar¹ (EC₅₀=0.95 μM). This finding led to preparation of several LHRH derivatives that incorporated amino acids with varied hydrophobicity at position 1 in conjunction with hydrophilic amino acid at position 2. Structure-activity analysis of the binding data in Table 5 indicated that substitution of lipophilic amino acids at position 1 in combination with a hydrophilic amino acid at position 2 usually provided analogs with increased binding potency. Substitution of Arg at position 2 in the analog of BRU-2666 afforded BRU-2813 with 30% more potency [0.33 μM (BRU-2813) vs 0.47 μM (BRU-2666)].

Thus BRU-2813 became a standard for the comparative binding study of other analogs involving various amino acid modifications. Lipophilic D-amino acids, such as Dnal2, at position 1 provided LHRH-II analogs with increased binding potency vs the analogs derived from the corresponding L-isomers; 0.33 μM (BRU-2813 with Dnal2) vs 0.62 μM (BRU-3051 with Nal2). However increased potency was not always observed when D-isomers were used instead of L-isomers at position 1. In the case of lipophilic amino acids such as Nal1, Tic, and Tpi, almost equipotent analogs were obtained whether D- or L-isomers were employed. A notable increase in potency was observed in the binding studies of the analogs with hydrophobic aromatic basic amino acids like Damfe4 (BRU-2757, EC₅₀=0.26 μM; BRU-3095, EC₅₀=0.29 μM) and Gufe4 (BRU-3058, EC₅₀=0.26 μM) employed at position either 1 or 2 or at both sites. A similar increase in potency was seen in the case of Dtpi, a conformationally restricted lipophilic imino acid, (BRU-3068, EC₅₀=0.24 μM) at position 1 in combination with Arg at position 2.

TABLE 5
LHRH Peptides with Modifications of Amino Acids at Positions 1 and 2

| | | Chelating | | | | | | | | | | | | | EC₅₀ |
|---|---|---|---|---|---|---|---|---|---|---|---|---|---|---|---|
| No | Seq # | Group | Linker | AA¹ | AA² | AA³ | AA⁴ | AA⁵ | AA⁶ | AA⁷ | AA⁸ | AA⁹ | AA¹⁰ | BRU # | μM |
| 1 | Seq007 | DO3A10CM | | Sar | H | W | S | H | r | W | Bpa4 | P | azaG | BRU- | 0.95 |
| | | | | | | | | | | | | | | 2443 | |
| 2 | Seq013 | DO3A10CM | | Dnal2 | H | W | S | H | r | W | Bpa4 | P | azaG | BRU- | 0.47 |
| | | | | | | | | | | | | | | 2666 | |
| 3 | Seq041 | DO3A10CM | | Gly | H | W | S | H | r | W | Bpa4 | P | azaG | BRU- | 1.18 |
| | | | | | | | | | | | | | | 2733 | |
| 4 | Seq042 | — | | Mephe | H | W | S | H | r | W | Bpa4 | P | azaG | BRU- | 0.15 |
| | | | | | | | | | | | | | | 2734 | |
| 5 | Seq043 | DO3A10CM | | Phe | H | W | S | H | r | W | Bpa4 | P | azaG | BRU- | 1.14 |
| | | | | | | | | | | | | | | 2735 | |
| 6 | Seq044 | DO3A10CM | | Meala | H | W | S | H | r | W | Bpa4 | P | azaG | BRU- | 1.42 |
| | | | | | | | | | | | | | | 2736 | |
| 7 | Seq045 | DO3A10CM | | Ambz4 | H | W | S | H | r | W | Bpa4 | P | azaG | BRU- | 1.40 |
| | | | | | | | | | | | | | | 2737 | |
| 8 | Seq053 | DO3A10CM | | Damfe4 | H | W | S | H | r | W | Bpa4 | P | azaG | BRU- | 0.26 |
| | | | | | | | | | | | | | | 2757 | |
| 9 | Seq067 | DO3A10CM | | EtGly | H | W | S | H | r | W | Bpa4 | P | azaG | BRU- | 0.21 |
| | | | | | | | | | | | | | | 2788 | |
| 10 | Seq080 | DO3A10CM | | Nal2 | H | W | S | H | r | W | Bpa4 | P | azaG | BRU- | 3.54 |
| | | | | | | | | | | | | | | 2809 | |
| 11 | Seq081 | DO3A10CM | | Dafe4 | H | W | S | H | r | W | Bpa4 | P | azaG | BRU- | 0.98 |
| | | | | | | | | | | | | | | 2810 | |
| 12 | Seq084 | DO3A10CM | | Dnal2 | R | W | S | H | r | W | Bpa4 | P | azaG | BRU- | 0.33 |
| | | | | | | | | | | | | | | 2813 | |
| 13 | Seq086 | DO3A10CM | | Dnal1 | H | W | S | H | r | W | Bpa4 | P | azaG | BRU- | 0.36 |
| | | | | | | | | | | | | | | 2869 | |
| 14 | Seq097 | DO3A10CM | | Dtyr | H | W | S | H | r | W | Bpa4 | P | azaG | BRU- | 10.0 |
| | | | | | | | | | | | | | | 2870 | |
| 15 | Seq108 | (DO3A10CM) | | Ac- | H | W | S | H | r | W | Bpa4 | P | azaG | BRU- | 1.38 |
| | | | | Amfe4 | | | | | | | | | | 2881 | |
| 16 | Seq110 | DO3A10CM | | Dphe | H | W | S | H | r | W | Bpa4 | P | azaG | BRU- | 2.00 |
| | | | | | | | | | | | | | | 2894 | |
| 17 | Seq155 | DO3A10CM | | Mednal2 | H | W | S | H | r | W | Bpa4 | P | azaG | BRU- | 0.76 |
| | | | | | | | | | | | | | | 3003 | |
| 18 | Seq127 | DO3A10CM | | Mednal2 | R | W | S | H | r | W | Bpa4 | P | azaG | BRU- | 0.58 |
| | | | | | | | | | | | | | | 2965 | |
| 19 | Seq028 | DO3A10CM | | Sar | 4ClPhe | W | S | H | r | W | Bpa4 | P | azaG | BRU- | 0.63 |
| | | | | | | | | | | | | | | 2719 | |
| 20 | Seq046 | DO3A10CM | | Sar | Phe | W | S | H | r | W | Bpa4 | P | azaG | BRU- | 1.22 |
| | | | | | | | | | | | | | | 2738 | |
| 21 | Seq049 | DO3A10CM | | Sar | His | W | S | H | r | W | Bpa4 | P | azaG | BRU- | 2.99 |
| | | | | | (1me) | | | | | | | | | 2741 | |
| 22 | Seq052 | DO3A10CM | | Sar | Amfe4 | W | S | H | r | W | Bpa4 | P | azaG | BRU- | 0.28 |
| | | | | | | | | | | | | | | 2756 | |
| 23 | Seq055 | DO3A10CM | | Sar | His | W | S | H | r | W | Bpa4 | P | azaG | BRU- | 10.0 |
| | | | | | (pime) | | | | | | | | | 2759 | |
| 24 | Seq064 | DO3A10CM | | Sar | Tha | W | S | H | r | W | Bpa4 | P | azaG | BRU- | 12.8 |
| | | | | | | | | | | | | | | 2768 | |
| 25 | Seq085 | DO3A10CM | | Sar | Tyr | W | S | H | r | W | Bpa4 | P | azaG | BRU- | 2.24 |
| | | | | | | | | | | | | | | 2814 | |
| 26 | Seq086 | DO3A10CM | | Sar | Dafe4 | W | S | H | r | W | Bpa4 | P | azaG | BRU- | 7.25 |
| | | | | | | | | | | | | | | 2815 | |
| 27 | Seq088 | DO3A10CM | | Sar | Arg | W | S | H | r | W | Bpa4 | P | azaG | BRU- | 1.70 |
| | | | | | | | | | | | | | | 2817 | |
| 28 | Seq089 | DO3A10CM | | Sar | Pal3 | W | S | H | r | W | Bpa4 | P | azaG | BRU- | 1.62 |
| | | | | | | | | | | | | | | 2818 | |
| 29 | Seq098 | DO3A10CM | | Sar | Lys | W | S | H | r | W | Bpa4 | P | azaG | BRU- | 10.0 |
| | | | | | | | | | | | | | | 2871 | |
| 30 | Seq014 | DO3A10CM | Gly | Pro | H | W | S | H | r | W | Bpa4 | P | azaG | BRU- | 7.29 |
| | | | | | | | | | | | | | | 2667 | |
| 31 | Seq026 | DO3A10CM | Gly | Sar | H | W | S | H | r | W | Bpa4 | P | azaG | BRU- | 1.55 |
| | | | | | | | | | | | | | | 2717 | |
| 32 | Seq037 | DO3A10CM | Gly | Thz | H | W | S | H | r | W | Bpa4 | P | azaG | BRU- | 3.15 |
| | | | | | | | | | | | | | | 2728 | |
| 33 | Seq062 | DO3A10CM | Gly | Dnal1 | H | W | S | H | r | W | Bpa4 | P | azaG | BRU- | 0.62 |
| | | | | | | | | | | | | | | 2766 | |
| 34 | Seq066 | DO3A10CM | Gly | Dnalg1 | H | W | S | H | r | W | Bpa4 | P | azaG | BRU- | 2.70 |
| | | | | | | | | | | | | | | 2770 | |
| 35 | Seq074 | DO3A10CM | Gly | Gly | H | W | S | H | r | W | Bpa4 | P | azaG | BRU- | 1.37 |
| | | | | | | | | | | | | | | 2795 | |
| 36 | Seq177 | DO3A10CM | — | Arg | R | W | S | H | r | W | Bpa4 | P | azaG | BRU- | 0.31 |
| | | | | | | | | | | | | | | 3050 | |
| 37 | Seq178 | DO3A10CM | — | Nal2 | R | W | S | H | r | W | Bpa4 | P | azaG | BRU- | 0.62 |
| | | | | | | | | | | | | | | 3051 | |
| 38 | Seq179 | DO3A10CM | Gly | Tic | R | W | S | H | r | W | Bpa4 | P | azaG | BRU- | 0.55 |
| | | | | | | | | | | | | | | 3052 | |
| 39 | Seq180 | DO3A10CM | Gly | Tpi | R | W | S | H | r | W | Bpa4 | P | azaG | BRU- | 0.32 |
| | | | | | | | | | | | | | | 3053 | |
| 40 | Seq181 | DO3A10CM | — | Dtyr | R | W | S | H | r | W | Bpa4 | P | azaG | BRU- | 0.43 |
| | | | | | | | | | | | | | | 3054 | |
| 41 | Seq182 | DO3A10CM | — | Atdc2 | R | W | S | H | r | W | Bpa4 | P | azaG | BRU- | 0.39 |
| | | | | | | | | | | | | | | 3055 | |
| 42 | Seq183 | DO3A10CM | — | Apsp | R | W | S | H | r | W | Bpa4 | P | azaG | BRU- | 0.44 |
| | | | | | | | | | | | | | | 3056 | |
| 43 | Seq184 | DO3A10CM | — | Qua3 | R | W | S | H | r | W | Bpa4 | P | azaG | BRU- | 0.38 |
| | | | | | | | | | | | | | | 3057 | |
| 44 | Seq190 | DO3A10CM | — | Datdc2 | R | W | S | H | r | W | Bpa4 | P | azaG | BRU- | 0.47 |
| | | | | | | | | | | | | | | 3063 | |
| 45 | Seq194 | DO3A10CM | Gly | Dtic | R | W | S | H | r | W | Bpa4 | P | azaG | BRU- | 0.51 |
| | | | | | | | | | | | | | | 3067 | |
| 46 | Seq195 | DO3A10CM | Gly | Dtpi | R | W | S | H | r | W | Bpa4 | P | azaG | BRU- | 0.24 |
| | | | | | | | | | | | | | | 3068 | |
| 47 | Seq196 | DO3A10CM | — | Thy | R | W | S | H | r | W | Bpa4 | P | azaG | BRU- | 0.38 |
| | | | | | | | | | | | | | | 3069 | |
| 48 | Seq197 | DO3A10CM | — | Bip | R | W | S | H | r | W | Bpa4 | P | azaG | BRU- | 0.28 |
| | | | | | | | | | | | | | | 3070 | |
| 49 | Seq198 | DO3A10CM | — | Dbpa4 | R | W | S | H | r | W | Bpa4 | P | azaG | BRU- | 0.31 |
| | | | | | | | | | | | | | | 3071 | |
| 50 | Seq201 | DO3A10CM | — | Cafe4 | R | W | S | H | r | W | Bpa4 | P | azaG | BRU- | 2.00 |
| | | | | | | | | | | | | | | 3092 | |
| 51 | Seq202 | DO3A10CM | — | Pstr4 | R | W | S | H | r | W | Bpa4 | P | azaG | BRU- | 17.00 |
| | | | | | | | | | | | | | | 3093 | |
| 52 | Seq203 | DO3A10CM | — | Ampha4 | R | W | S | H | r | W | Bpa4 | P | azaG | BRU- | 0.35 |
| | | | | | | | | | | | | | | 3094 | |
| 53 | Seq204 | DO3A10CM | — | Damfe4 | Damfe4 | W | S | H | r | W | Bpa4 | P | azaG | BRU- | 0.29 |
| | | | | | | | | | | | | | | 3095 | |
| 54 | Seq176 | DO3A10CM | — | Dnal2 | Darg | W | S | H | r | W | Bpa4 | P | azaG | BRU- | 0.36 |
| | | | | | | | | | | | | | | 3049 | |
| 55 | Seq185 | DO3A10CM | — | Dnal2 | Gufe4 | W | S | H | r | W | Bpa4 | P | azaG | BRU- | 0.26 |
| | | | | | | | | | | | | | | 3058 | |
| 56 | Seq186 | DO3A10CM | — | Dnal2 | Ampa3 | W | S | H | r | W | Bpa4 | P | azaG | BRU- | 0.41 |
| | | | | | | | | | | | | | | 3059 | |
| 57 | Seq187 | DO3A10CM | — | Dnal2 | Ampg2 | W | S | H | r | W | Bpa4 | P | azaG | BRU- | 0.34 |
| | | | | | | | | | | | | | | 3060 | |

BRU Nos. 2733, 2735, 2736, 2737, 2788, 2809, 2810, 2870, 2881, 2894, 3003, 2965, 2719, 2738, 2741, 2756, 2759, 2768, 2814, 2815, 2817, 2818, 2871, 2667, 2717, 2728, 2766, 2770, 2795, 3051, 3052, 3067, 3092 and 3093 in Table 5 immediately above correspond, respectively, to SEQ ID NOs: 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146 and 147 in the Sequence Listing.

These observations led to the conclusion that the basic lipophilic amino acids at positions 1 and 2, in particular those with a guanidine moiety, would yield high-affinity-binding LHRH-II analogs with increased biological potency, and this increased potency might be attributed to the conformational stabilizing effect of the basic moiety by charge-interaction or H-bonding with the receptor. Conversely, repulsive charge interaction of acid moieties such as —COOH and —OPO₃H might explain the low binding affinities of analogs BRU-3092 (EC₅₀=2.00 μM) with Cafe4 and BRU-3093 (EC₅₀=17.00 μM) with Pstr4 at position 1.

Modifications of AzaGly¹⁰ at the C-terminus

While the LHRH-free acid exhibited very low potency in vitro, replacement with alkyl amines at position 10 provided nonapeptide alkyl amides with more significant binding potency. In our studies, peptides BRU-2968 ([Pro⁹-NHCH₂CH₂OCH₂CH₂NH₂]BRU-2813) and BRU-2969 ([Pro⁹-Gly¹⁰-Arg-NH₂]BRU-2813) showed increased potency compared to BRU-2813, which contains Pro⁹-azaGly¹⁰-amide. Likewise, LHRH-II analogs with AzaGly¹⁰ modifications having free amine or guanidine functionalities with more basicity and/or in conjunction with lipophilicity (aliphatic/aromatic character) showed in general comparable binding to that of BRU-2813 with EFO-27 cells.

TABLE 6
LHRH Peptides with Modification at the C-terminus

| | | Chelating | | | | | | | | | | | | EC₅₀ |
|---|---|---|---|---|---|---|---|---|---|---|---|---|---|---|
| No | Seq. # | Group | AA¹ | AA² | AA³ | AA⁴ | AA⁵ | AA⁶ | AA⁷ | AA⁸ | AA⁹ | AA¹⁰ | BRU # | μM |
| 1 | Seq128 | DO3A10CM | Dnal2 | R | W | S | H | r | W | Bpa4 | P | Az34m3buo | 2967 | 0.44 |
| 2 | Seq129 | DO3A10CM | Dnal2 | R | W | S | H | r | W | Bpa4 | P | Da15o3pt | 2968 | 0.24 |
| 3 | Seq130 | DO3A10CM | Dnal2 | R | W | S | H | r | W | Bpa4 | P | Gly-Arg- | 2969 | 0.24 |
| | | | | | | | | | | | | NH₂ | | |
| 4 | Seq131 | DO3A10CM | Dnal2 | R | W | S | H | r | W | Bpa4 | P | Aeh2 | 2970 | 0.51 |
| 5 | Seq132 | DO3A10CM | Dnal2 | R | W | S | H | r | W | Bpa4 | P | Gly-Gln | 2971 | 0.49 |
| | | | | | | | | | | | | NH₂ | | |
| 6 | Seq133 | DO3A10CM | Dnal2 | R | W | S | H | r | W | Bpa4 | P | Gly-Abt1h4 | 2978 | 2.00 |
| 7 | Seq134 | DO3A10CM | Dnal2 | R | W | S | H | r | W | Bpa4 | P | Gly-Abn | 2979 | 0.99 |
| 8 | Seq135 | DO3A10CM | Dnal2 | R | W | S | H | r | W | Bpa4 | P | Gly-Phe- | 2980 | 0.97 |
| | | | | | | | | | | | | NH₂ | | |
| 9 | Seq136 | DO3A10CM | Dnal2 | R | W | S | H | r | W | Bpa4 | P | Ae | 2981 | 0.97 |
| 10 | Seq137 | DO3A10CM | Dnal2 | R | W | S | H | r | W | Bpa4 | P | Aprp1h3 | 2982 | 1.13 |
| 11 | Seq138 | DO3A10CM | Dnal2 | R | W | S | H | r | W | Bpa4 | P | Pheol | 2983 | 0.45 |
| 12 | Seq139 | DO3A10CM | Dnal2 | R | W | S | H | r | W | Bpa4 | P | Gua | 2984 | 0.40 |
| 13 | Seq140 | DO3A10CM | Dnal2 | R | W | S | H | r | W | Bpa4 | P | Alguao3pt | 2985 | 0.46 |
| 14 | Seq141 | DO3A10CM | Dnal2 | R | W | S | H | r | W | Bpa4 | P | Gly-Abt1h4 | 2986 | 1.66 |
| 15 | Seq142 | DO3A10CM | Dnal2 | R | W | S | H | r | W | Bpa4 | P | Gly-Aprp1h3 | 2987 | 2.30 |
| 16 | Seq143 | DO3A10CM | Dnal2 | R | W | S | H | r | W | Bpa4 | P | Gly-Abn | 2988 | 0.65 |
| 17 | Seq144 | DO3A10CM | Dnal2 | R | W | S | H | r | W | Bpa4 | P | Gly- | 2989 | 1.13 |
| | | | | | | | | | | | | Az23(py2) | | |
| | | | | | | | | | | | | 2po-Ap | | |
| 18 | Seq156 | DO3A10CM | Dnal2 | R | W | S | H | r | W | Bpa4 | P | Gly-NH₂ | 3005 | 0.66 |
| 19 | Seq157 | DO3A10CM | Dnal2 | R | W | S | H | r | W | Bpa4 | P | Ap | 3006 | 0.42 |
| 20 | Seq158 | — | Sar | R | W | S | H | r | W | Bpa4 | P | Da15o3pt | 3007 | 0.22 |
| 21 | Seq159 | DO3A10CM | Dnal2 | R | W | S | H | r | W | Bpa4 | P | Az23po- | 3019 | 0.90 |
| | | | | | | | | | | | | Dabt14 | | |
| 22 | Seq160 | DO3A10CM | Dnal2 | R | W | S | H | r | W | Bpa4 | P | Mo2abn | 3020 | 0.45 |
| 23 | Seq161 | DO3A10CM | Dnal2 | R | W | S | H | r | W | Bpa4 | P | Az23m2po- | 3021 | 0.50 |
| | | | | | | | | | | | | NH₂ | | |
| 24 | Seq162 | DO3A10CM | Dnal2 | R | W | S | H | r | W | Bpa4 | P | Az23po- | 3022 | 0.71 |
| | | | | | | | | | | | | Da15o3pt | | |
| 25 | Seq175 | DO3A10CM | Dnal2 | R | W | S | H | r | W | — | — | — | 3046 | 15.9 |

From the binding data shown in Table 6 it appears that the terminal azaglycine amide is not essential for high potency and the total chain length and the basic character of the Pro⁹-amide substitution plays an important role in the binding affinity of these analogs on ovarian cancer cells. Therefore it is possible to suggest, in accordance with earlier reports, that the introduction of these Pro⁹-alkylamide moieties might increase the duration of action of these analogs by virtue of their greater resistance to post-proline enzymatic proteolysis.³⁷

### Modifications at Position 6: Efforts to Increase Hydrophilicity

The change of the position-6 residue from an L-amino acid to a D-amino acid yielded an LHRH analog (e.g., [D-Ala⁶]LHRH-II)⁵⁶ with a potency approximately 4 times greater than that of LHRH-II both in vitro and in ovariectomized rats.^{57,58} Likewise, in our studies, Darg substitution at position 6 in combination with Ac-Sar and Bpa4 at position 1 and 8 respectively, provided an analog, [Sar¹, Darg⁶, Bpa4⁸, azaGly¹⁰]LHRH-II (BRU-2441, EC₅₀=0.14 μM) showing favorable in vitro binding in EFO-27 cells. This prompted preparation of a series of DO3A10CM-metal chelate containing compounds with a D-amino acid at position 6, including Dala and Darg derivatives with modified guanidine moieties (see Table 7). Interestingly, LHRH-II analogs, BRU-2729 with Dcit⁶ (EC₅₀=14.24 μM), BRU-2880 with Dharg(Et)₂⁶ (EC₅₀=8.02 μM), and BRU-2893 with Dharg⁶ (EC₅₀=2.60 μM) respectively showed binding in vitro that was 15, 8.4 and 2.7 times lower than the corresponding similarly constituted BRU-2443 with Darg⁶ (EC₅₀=0.95 μM). This result suggested the need for a D-amino acid of the correct basicity placed at a specific distance from the peptide backbone with less steric crowding to enhance the potency during receptor interaction. The increased biological potency of Darg⁶ might be attributed to the conformational stabilizing effect at the β-II′ type turn involving -See⁴-His⁵-Darg⁶-Trp⁷- which was favorable for the charge-interaction or H-bonding at the receptor. When Darg⁶ was replaced by Btd⁶, a conformationally restricted bicyclic amino acid, the resulting analog BRU-3000 (EC₅₀=4.23 μM) showed 4.5 times lesser binding efficacy than that of BRU-2443 with Darg⁶ (EC₅₀=0.95 μM); this might due to be the disruption of the β-II′ type bend.

TABLE 7
LHRH Peptides with Modification of the Amino Acid at Position 6

| | | Chelating | | | | | | | | | | | | EC₅₀ |
|---|---|---|---|---|---|---|---|---|---|---|---|---|---|---|
| No | Seq # | Group | AA¹ | AA² | AA³ | AA⁴ | AA⁵ | AA⁶ | AA⁷ | AA⁸ | AA⁹ | AA¹⁰ | BRU # | μM |
| 1 | Seq001 | — | pGlu | H | W | S | H | Dlys | W | Tyr | P | azaG | 2437 | 8.54 |
| 2 | Seq009 | — | pGlu | H | W | S | H | Darg | W | Tyr | P | azaG | 2477 | 0.74 |
| 3 | Seq003 | — | Sar | H | W | S | H | Darg | W | Tyr | P | azaG | 2439 | 0.25 |
| 4 | Seq005 | — | Sar | H | W | S | H | Darg | W | Bpa4 | P | azaG | 2441 | 0.14 |
| 5 | Seq007 | DO3A10CM | Sar | H | W | S | H | Darg | W | Bpa4 | P | azaG | 2443 | 0.95 |
| 6 | Seq018 | DO3A10CM | Sar | H | W | S | H | Dlys(Nic) | W | Bpa4 | P | azaG | 2671 | 7.20 |
| 7 | Seq019 | DO3A10CM | Sar | H | W | S | H | Dala | W | Bpa4 | P | azaG | 2672 | 14.00 |
| 8 | Seq020 | DO3A10CM | Sar | H | W | S | H | Dpal3 | W | Bpa4 | P | azaG | 2673 | 12.33 |
| 9 | Seq021 | DO3A10CM | Sar | H | W | S | H | Dlys(Nic) | W | Tyr | P | azaG | 2674 | 22.64 |
| 10 | Seq038 | DO3A10CM | Sar | H | W | S | H | Dcit | W | Bpa4 | P | azaG | 2729 | 14.24 |
| 11 | Seq040 | DO3A10CM | Sar | H | W | S | H | Dtrp | W | Bpa4 | P | azaG | 2731 | 1.36 |
| 12 | Seq057 | DO3A10CM | Sar | H | W | S | H | Dser | W | Bpa4 | P | azaG | 2761 | 13.57 |
| 13 | Seq107 | DO3A10CM | Sar | H | W | S | H | Dharg(Et)₂ | W | Bpa4 | P | azaG | 2880 | 8.02 |
| 14 | Seq109 | DO3A10CM | Sar | H | W | S | H | Dharg | W | Bpa4 | P | azaG | 2893 | 2.60 |
| 15 | Seq109 | DO3A10CM | Sar | H | W | S | H | Btd | W | Bpa4 | P | azaG | 3000 | 4.23 |

BRU Nos. 2671, 2672, 2673, 2674, 2729, 2731, 2761, 2880, 2893 and 3000 in Table 7 immediately above correspond, respectively, to SEQ ID NOs: 148, 149, 150, 151, 152, 153, 154, 155, 156 and 157 in the Sequence Listing.

### Modifications at Position 1: Effect of N-Substitution

The effect of N-methylation on in vitro potency of several LHRH agonists and antagonists has been reported^{49,50} to cause significant reduction in binding affinity and in some cases changed the compounds from agonists to antagonists. Hence, in order to study the effect of N-substituted amino acid at position 1, LHRH-II analogs (Table 8) with variously N-substituted Gly at position 1 were synthesized and in vitro binding was performed on EFO-27 cells. To introduce the N-substituted-Gly into the peptide sequence during the construction of the sequence by the automated standard solid-phase method, the peptoid synthesis approach⁴³ was employed. This technique promptly enabled the appendage of N-(substituted)glycine from readily available bromoacetic acid and various primary amines in the course of the chain elongation. The addition of N-(substituted)glycines consisted of an acylation step with bromoacetic acid and a nucleophilic displacement step involving displacement of bromine by a wide variety of primary amines After the introduction of the N-(substituted)glycine to the resultant secondary amine, glycine was coupled to facilitate the ensuing DO3A10CM coupling, which proceeded to completion.

In general, along the lines of the earlier reports,^{59,60} losses in binding affinity were observed in this series of LHRH-II peptides; with HN(CH₂CH₂COOH)Gly (BRU-2875, EC₅₀=8.70 μM) at the extreme indicating the deleterious effect of a —COOH moiety to the receptor interaction. Likewise a reduction (˜2×) in binding potency was seen in the case of the LHRH analog with N-methyl-2-naphthyl-D-alanine (Mednal2) at position 1, 0.58 μM (BRU-2965 with Mednal2) vs 0.33 μM (BRU-2813 with Dnal2).

TABLE 8
LHRH Peptides with a N-Substituted Amino Acid at Position 1 and Gly as linker

| | | | | | | | | | | | | | | | EC₅₀ |
|---|---|---|---|---|---|---|---|---|---|---|---|---|---|---|---|
| No | Seq # | Chelating Group | Linker | AA¹ | AA² | AA³ | AA⁴ | AA⁵ | AA⁶ | AA⁷ | AA⁸ | AA⁹ | AA¹⁰ | BRU# | μM |
| 1 | Seq051 | DO3A10CM | Gly | Mephe | H | W | S | H | R | W | Bpa4 | P | azaG | 2755 | 1.29 |
| 2 | Seq068 | DO3A10CM | Gly | Etgly | H | W | S | H | R | W | Bpa4 | P | azaG | 2789 | 5.12 |
| 3 | Seq069 | DO3A10CM | Gly | Ipgly | H | W | S | H | R | W | Bpa4 | P | azaG | 2790 | 3.30 |
| 4 | Seq070 | DO3A10CM | Gly | Bugly | H | W | S | H | R | W | Bpa4 | P | azaG | 2791 | 4.03 |
| 5 | Seq071 | DO3A10CM | Gly | Bzgly | H | W | S | H | R | W | Bpa4 | P | azaG | 2792 | 2.27 |
| 6 | Seq072 | DO3A10CM | Gly | Mpgly | H | W | S | H | R | W | Bpa | P | azaG | 2793 | 1.34 |
| 7 | Seq073 | DO3A10CM | Gly | Prgly | H | W | S | H | R | W | Bpa4 | P | azaG | 2794 | 1.30 |
| 8 | Seq075 | DO3A10CM | Gly | Mogly | H | W | S | H | R | W | Bpa4 | P | azaG | 2796 | 0.34 |
| 9 | Seq076 | DO3A10CM | Gly | Hpgly | H | W | S | H | R | W | Bpa4 | P | azaG | 2797 | 0.45 |
| 10 | Seq099 | DO3A10CM | Gly | Chgly | H | W | S | H | R | W | Bpa4 | P | azaG | 2872 | 2.90 |
| 12 | Seq100 | DO3A10CM | Gly | Hegly | H | W | S | H | R | W | Bpa4 | P | azaG | 2873 | 1.33 |
| 13 | Seq101 | DO3A10CM | Gly | Apgly | H | W | S | H | R | W | Bpa4 | P | azaG | 2874 | 1.97 |
| 14 | Seq102 | DO3A10CM | Gly | Cegly | H | W | S | H | R | W | Bpa4 | P | azaG | 2875 | 8.70 |
| 15 | Seq103 | DO3A10CM | Gly | Ahgly | H | W | S | H | R | W | Bpa4 | P | azaG | 2876 | 0.50 |
| 16 | Seq104 | DO3A10CM | Gly | Chmgly | H | W | S | H | R | W | Bpa4 | P | azaG | 2877 | 0.90 |
| 17 | Seq105 | DO3A10CM | Gly | Tdgly | H | W | S | H | R | W | Bpa4 | P | azaG | 2878 | 4.48 |
| 18 | Seq106 | DO3A10CM | Gly | Iegly | H | W | S | H | R | W | Bpa4 | P | azaG | 2879 | 1.03 |
| 19 | Seq155 | DO3A10CM | — | Mednal2 | H | W | S | H | R | W | Bpa4 | P | azaG | 3003 | 0.76 |
| 20 | Seq127 | DO3A10CM | — | Mednal2 | R | W | S | H | R | W | Bpa4 | P | azaG | 2965 | 0.58 |

BRU Nos. 2755, 2789, 2790, 2791, 2792, 2793, 2794, 2872, 2873, 2874, 2875, 2877, 2878 and 2879 in Table 8 immediately above correspond, respectively, to SEQ ID NOs: 158, 159, 160, 161, 162, 163, 164, 165, 166, 167, 168, 169, 170 and 171 in the Sequence Listing.

### Effect of Modifications at Position 9

LHRH-II analogs where Pro⁹ was replaced with 4-substituted L-Pro derivatives having functionalities like —OH, F, phenyl and NH₂ (cis and trans) were prepared (Table 9) to study the effect of the conformational change on the binding efficacy. Peptides with azetidine carboxylic acid (Aze, BRU-2993) and pipecolic acid (Pip, BRU-2996) replacing Pro⁹ were also made to discern the effect of the ring size on the conformation during receptor interaction. Each of these residues, either with hydrophilic substitution on Pro⁹ or altered ring size at position 9 produced active analogs, albeit with little change in potency.

TABLE 9
LHRH Peptides with Modification of the Amino Acid at Position 9

| | | Chelating | | | | | | | | | | | | EC₅₀ |
|---|---|---|---|---|---|---|---|---|---|---|---|---|---|---|
| No. | Seq # | Group | AA¹ | AA² | AA³ | AA⁴ | AA⁵ | AA⁶ | AA⁷ | AA⁸ | AA⁹ | AA¹⁰ | BRU # | μM |
| 1 | Seq013 | DO3A10CM | Dnal2 | R | W | S | H | r | W | Bpa4 | Pro | azaG | 2813 | 0.33 |
| 2 | Seq114 | DO3A10CM | Dnal2 | R | W | S | H | r | W | Bpa4 | Hypt4 | azaG | 2952 | 0.28 |
| 3 | Seq115 | DO3A10CM | Dnal2 | R | W | S | H | r | W | Bpa4 | Ppt4 | azaG | 2953 | 0.65 |
| 4 | Seq145 | DO3A10CM | Dnal2 | R | W | S | H | r | W | Bpa4 | Aze | azaG | 2993 | 0.38 |
| 5 | Seq146 | DO3A10CM | Dnal2 | R | W | S | H | r | W | Bpa4 | Flp4 | azaG | 2994 | 0.60 |
| 6 | Seq147 | DO3A10CM | Dnal2 | R | W | S | H | r | W | Bpa4 | Ampt4 | azaG | 2995 | 0.41 |
| 7 | Seq148 | DO3A10CM | Dnal2 | R | W | S | H | r | W | Bpa4 | Pip | azaG | 2996 | 0.35 |
| 8 | Seq189 | DO3A10CM | Dnal2 | R | W | S | H | r | W | Bpa4 | Ampc4 | azaG | 3062 | 0.32 |
| 9 | Seq199 | DO3A10CM | Dnal2 | R | W | S | H | r | W | Bpa4 | Thz | azaG | 3072 | 0.16 |
| 10 | Seq175 | DO3A10CM | Dnal2 | R | W | S | H | r | W | — | — | — | 3046 | 15.9 |
| 11 | Seq191 | DO3A10CM | Dnal2 | R | W | S | H | r | W | Bpa4 | — | — | 3064 | 0.67 |

Substitution of a bulky phenyl group on the Pro⁹ (Ppt4⁹, BRU-2953, EC₅₀=0.65 μM), distorted the conformation around the C-terminus and reduced the binding by a factor of 2 by comparison with derivative with Pro⁹ (BRU-2813, EC₅₀=0.33 μM), Very interestingly, with L-thiazolidine-4-carboxylic acid (Thz, thiaproline) (Thz⁹, BRU-3072, EC₅₀=0.16 μM), replacing Pro⁹ binding was ˜2.5 fold improved vs that of the corresponding Pro⁹ analog. In general, fragments or truncated (deletion) analogs of LHRH-II without Pro⁹ possessed very low LHRH potency. BRU-3064 (which was found to be a metabolite of BRU-2813) is a notable exception.

### Effect of Modifications at Position 4

Peptide analogs where Ser⁴ was replaced by amino acids with functional groups like —COOH (Asp), —CONH₂ (Asn), —NH₂ (Dpr, Amfe4) and —SCH₃ (Met) or with a lipophilic moiety (Leu and Trp) were prepared and their binding on EFO-27 cells are given in Table 10. Analysis of these binding data revealed the requirement of a basic amino acid preferably with increased lipophilicity (Amfe4) at position 4 to provide analogs with high potency.

TABLE 10
LHRH Peptides with Modification of Amino Acid at Position 4

| | | Chelating | | | | | | | | | | | | EC₅₀ |
|---|---|---|---|---|---|---|---|---|---|---|---|---|---|---|
| No | Seq. # | Group | AA¹ | AA² | AA³ | AA⁴ | AA⁵ | AA⁶ | AA⁷ | AA⁸ | AA⁹ | AA¹⁰ | BRU # | μM |
| 1 | Seq013 | DO3A10CM | Dnal2 | R | W | Ser | H | r | W | Bpa4 | P | azaG | 2813 | 0.33 |
| 2 | Seq116 | DO3A10CM | Dnal2 | R | W | Asp | H | r | W | Bpa4 | P | azaG | 2954 | 4.20 |
| 3 | Seq117 | DO3A10CM | Dnal2 | R | W | Dpr | H | r | W | Bpa4 | P | azaG | 2955 | 0.41 |
| 4 | Seq118 | DO3A10CM | Dnal2 | R | W | Asn | H | r | W | Bpa4 | P | azaG | 2956 | 0.28 |
| 5 | Seq119 | DO3A10CM | Dnal2 | R | W | Pal4 | H | r | W | Bpa4 | P | azaG | 2957 | 0.84 |
| 6 | Seq126 | DO3A10CM | Dnal2 | R | W | Met | H | r | W | Bpa4 | P | azaG | 2964 | 0.22 |
| 7 | Seq219 | DO3A10CM | Dnal2 | R | W | Leu | H | r | W | Bpa4 | P | azaG | 3113 | 1.13 |
| 8 | Seq220 | DO3A10CM | Dnal2 | R | W | Trp | H | r | W | Bpa4 | P | azaG | 3114 | 11.50 |
| 9 | Seq221 | DO3A10CM | Dnal2 | R | W | Amfe4 | H | r | W | Bpa4 | P | azaG | 3115 | 0.15 |

BRU Nos. 2954, 2957, 3113 and 3114 in Table 10 immediately above correspond, respectively, to SEQ ID NOs: 172, 173, 174 and 175 in the Sequence Listing.

Interestingly, aspartic acid with a pendant —COO⁻ group provided a peptide BRU-2954 (EC₅₀=4.20 μM) with binding efficacy ˜13 times lower than that of the standard analog BRU-2813 (EC₅₀=0.33 μM) suggesting a repulsive interaction of the carboxylate function with the receptor. Conversely, methionine at position 4 provided BRU-2964 (EC₅₀=0.22 μM) with 30% more potency in vitro.

### Effect of Modifications at Position 5

Table 11 provides the LHRH-II analogs where His⁵ is replaced by amino acids of varied basicity to explore the consequences of such replacement on the in vitro binding potency. Substitution of Tha (L-4-thiazolylalanine) with a similar aromatic ring (NH replaced by S) like His, at position 5 provided a LHRH-II analog BRU-2769 (EC₅₀=7.34 μM) and showed binding potency in vitro ˜8× lower than the corresponding similarly constituted BRU-2443 with His^{5 (EC}₅₀=0.95 μM). Likewise an even greater reduction in potency was seen for other analogs with Tha⁵, BRU-2739 (EC₅₀=5.28 μM), or Tha²-Tha⁵, BRU-2762 (EC₅₀=15.03 μM). This investigation of analogs with Tha at positions 2 and 5, demonstrated the importance of an amino acid with a basic side chain (such as Orn or Arg) at positions 2 and 5 for increased in vitro potency. The lack of a basic amino acid at position 5 in the following analogs BRU-2668 (Tyr⁵, EC₅₀=3.47 μM), BRU-3029 (Leu⁵, EC₅₀=1.21 μM) and BRU-3030 (Cit⁵, EC₅₀=0.93 μM), led to low in vitro binding potency.

TABLE 11
LHRH Peptides with Modification Amino Acid at Position 5

| | | Chelating | | | | | | | | | | | | EC₅₀ |
|---|---|---|---|---|---|---|---|---|---|---|---|---|---|---|
| No | Seq # | Group | AA¹ | AA² | AA³ | AA⁴ | AA⁵ | AA⁶ | AA⁷ | AA⁸ | AA⁹ | AA¹⁰ | BRU # | μM |
| 1 | Seq007 | DO3A10CM | Sar | H | W | S | His | r | W | Bpa4 | P | azaG | 2443 | 0.95 |
| 2 | Seq015 | DO3A10CM | Sar | H | W | S | Tyr | r | W | Bpa4 | P | azaG | 2668 | 3.47 |
| 3 | Seq017 | DO3A10CM | Sar | H | W | S | Tyr | r | Leu | Bpa4 | P | azaG | 2670 | 8.84 |
| 4 | Seq047 | DO3A10CM | Sar | His | W | S | Tha | r | W | Bpa4 | P | azaG | 2739 | 5.28 |
| | | | | (pime) | | | | | | | | | | |
| 5 | Seq048 | DO3A10CM | Sar | — | W | S | Tha | r | W | Bpa4 | P | azaG | 2740 | 5.53 |
| 6 | Seq058 | DO3A10CM | Sar | Tha | W | S | Tha | r | W | Bpa4 | P | azaG | 2762 | 15.03 |
| 7 | Seq065 | DO3A10CM | Sar | H | W | S | Tha | r | W | Bpa4 | P | azaG | 2769 | 7.34 |
| 8 | Seq013 | DO3A10CM | Dnal2 | H | W | S | His | r | W | Bpa4 | P | azaG | 2666 | 0.47 |
| 9 | Seq084 | DO3A10CM | Dnal2 | R | W | S | His | r | W | Bpa4 | P | azaG | 2813 | 0.33 |
| 10 | Seq120 | DO3A10CM | Dnal2 | R | W | S | Tha | r | W | Bpa4 | P | azaG | 2958 | 0.71 |
| 11 | Seq121 | DO3A10CM | Dnal2 | R | W | S | Arg | r | W | Bpa4 | P | azaG | 2959 | 0.25 |
| 12 | Seq122 | DO3A10CM | Dnal2 | R | W | S | Fur3ala | r | W | Bpa4 | P | azaG | 2960 | 0.40 |
| 13 | Seq123 | DO3A10CM | Dnal2 | R | W | S | Orn | r | W | Bpa4 | P | azaG | 2961 | 0.35 |
| 14 | Seq153 | DO3A10CM | Dnal2 | R | W | S | Arg | Dtrp | W | Bpa4 | P | azaG | 3001 | 3.28 |
| 15 | Seq166 | DO3A10CM | Dnal2 | R | W | S | Ala | r | W | Bpa4 | P | azaG | 3028 | 1.09 |
| 16 | Seq167 | DO3A10CM | Dnal2 | R | W | S | Leu | r | W | Bpa4 | P | azaG | 3029 | 1.21 |
| 17 | Seq168 | DO3A10CM | Dnal2 | R | W | S | Cit | r | W | Bpa4 | P | azaG | 3030 | 0.93 |

BRU Nos. 2668, 2670, 2739, 2740, 2762, 2769, 2958, 3001, 3028, 3029 and 3030 in Table 11 immediately above correspond, respectively, to SEQ ID NOs: 176, 177, 178, 179, 180, 181, 182, 183, 184, 185 and 186 in the Sequence Listing.

### Effect of Modifications at Position 3

To determine the importance of hydrophobicity at position 3, LHRH-II analogs (Table 12) with amino acids (Nal1, Nal2, Phe, Amfe4, Leu and Dtrp) with varied lipophilicity and amino acids (Arg, Glu and Pa13) with hydrophilic functionality at position 3 were prepared. Binding data (Table 12) revealed that amino acids with increased lipophilicity (naphthylalanines) and amino acids with high basicity (Arg, Amfe4) provided analogs with moderate binding akin to that of the standard compound, BRU-2813. This could be attributed to the steric effect of a bulky aromatic ring in the case of naphthylalanines and charge-interaction or H-bonding of the basic moiety in the case Arg. Again, at position 4, glutaric acid provided a peptide BRU-3110 (EC₅₀=2.60 μM) with binding efficacy ˜8× lower than that of the standard analog BRU-2813 (EC₅₀=0.33 μM) suggesting a repulsive interaction of the presumed carboxylate function with the receptor.

TABLE 12
LHRH Peptides with Modification of Amino Acid at Position 3

| | | Chelating | | | | | | | | | | | | EC₅₀ |
|---|---|---|---|---|---|---|---|---|---|---|---|---|---|---|
| No | Seq. # | Group | AA¹ | AA² | AA³ | AA⁴ | AA⁵ | AA⁶ | AA⁷ | AA⁸ | AA⁹ | AA¹⁰ | BRU # | μM |
| 1 | Seq084 | DO3A10CM | Dnal2 | R | Trp | S | H | r | W | Bpa4 | P | azaG | 2813 | 0.33 |
| 2 | Seq210 | DO3A10CM | Dnal2 | R | Nal1 | S | H | r | W | Bpa4 | P | azaG | 3104 | 0.25 |
| 3 | Seq211 | DO3A10CM | Dnal2 | R | Nal2 | S | H | r | W | Bpa4 | P | azaG | 3105 | 0.22 |
| 4 | Seq212 | DO3A10CM | Dnal2 | R | Phe | S | H | r | W | Bpa4 | P | azaG | 3106 | 0.43 |
| 5 | Seq213 | DO3A10CM | Dnal2 | R | Amfe4 | S | H | r | W | Bpa4 | P | azaG | 3107 | 0.38 |
| 6 | Seq214 | DO3A10CM | Dnal2 | R | Leu | S | H | r | W | Bpa4 | P | azaG | 3108 | 0.56 |
| 7 | Seq215 | DO3A10CM | Dnal2 | R | Pal3 | S | H | r | W | Bpa4 | P | azaG | 3109 | 0.73 |
| 8 | Seq216 | DO3A10CM | Dnal2 | R | Glu | S | H | r | W | Bpa4 | P | azaG | 3110 | 2.60 |
| 9 | Seq217 | DO3A10CM | Dnal2 | R | Arg | S | H | r | W | Bpa4 | P | azaG | 3111 | 0.25 |
| 10 | Seq218 | DO3A10CM | Dnal2 | R | Dtrp | S | H | r | W | Bpa4 | P | azaG | 3112 | 0.85 |

BRU Nos. 3108, 3109, 3110 and 3112 in Table 12 immediately above correspond, respectively, to SEQ ID NOs: 187, 188, 189 and 190 in the Sequence Listing.

### Effect of Linker Length

LHRH-II peptides shown in Table 13 were synthesized to explore the effect of a linker between the N-terminus amino acid (AA¹) and the metal chelating agent, DO3A10CM on the binding efficacy. Insertion of Gly as a linker reduced the in vitro potency, irrespective of the nature of AA¹; a similar effect was observed to a greater extent in the case of 8-amino-3,6-dioxaoctanoic acid (Adoa) as a linker.

TABLE 13
LHRH Peptides with a Linker between AA¹ and DO3A10CM

| | | Chelating | | | | | | | | | | | | | EC₅₀ |
|---|---|---|---|---|---|---|---|---|---|---|---|---|---|---|---|
| No | Seq # | Group | Linker | AA¹ | AA² | AA³ | AA⁴ | AA⁵ | AA⁶ | AA⁷ | AA⁸ | AA⁹ | AA¹⁰ | BRU # | μM |
| 1 | Seq007 | DO3A10CM | — | Sar | H | W | S | H | r | W | Bpa4 | P | azaG | 2443 | 0.95 |
| 2 | Seq026 | DO3A10CM | Gly | Sar | H | W | S | H | r | W | Bpa4 | P | azaG | 2717 | 1.53 |
| 3 | Seq036 | DO3A10CM | Ambz4 | Sar | H | W | S | H | r | W | Bpa4 | P | azaG | 2727 | 0.65 |
| 4 | Seq025 | DO3A10CM | Gly-Abz4 | Sar | H | W | S | H | r | W | Bpa4 | P | azaG | 2696 | 0.65 |
| 5 | Seq111 | DO3A10CM | Adoa | Sar | H | W | S | H | r | W | Bpa4 | P | azaG | 2696 | 3.30 |
| 6 | Seq112 | DO3A10CM | Adoa- | Sar | H | W | S | H | r | W | Bpa4 | P | azaG | 2696 | 3.20 |
| | | | Adoa | | | | | | | | | | | | |
| 7 | Seq071 | DO3A10CM | Gly | Dnal1 | H | W | S | H | r | W | Bpa4 | P | azaG | 2792 | 0.65 |
| 8 | Seq086 | DO3A10CM | — | Dnal1 | H | W | S | H | r | W | Bpa4 | P | azaG | 2869 | 0.36 |
| 9 | Seq041 | DO3A10CM | | Gly | H | W | S | H | r | W | Bpa4 | P | azaG | 2733 | 1.18 |
| 10 | Seq074 | DO3A10CM | Gly | Gly | H | W | S | H | r | W | Bpa4 | P | azaG | 2795 | 1.37 |
| 11 | Seq084 | DO3A10CM | — | Dnal2 | R | W | S | H | r | W | Bpa4 | P | azaG | 2813 | 0.33 |
| 12 | Seq154 | DO3A10CM | Gly-Abz4 | Dnal2 | R | W | S | H | r | W | Bpa4 | P | azaG | 3002 | 0.50 |
| 13 | Seq205 | DO3A10CM | Dap | Dnal2 | R | W | S | H | r | W | Bpa4 | P | azaG | 3096 | 0.37 |
| 14 | Seq206 | DO3A10CM | Lys | Dnal2 | R | W | S | H | r | W | Bpa4 | P | azaG | 3097 | 0.45 |
| 15 | Seq208 | DO3A10CM | Dlys | Dnal2 | R | W | S | H | r | W | Bpa4 | P | azaG | 3099 | 0.44 |
| 16 | Seq129 | DO3A10CM | — | Dnal2 | R | W | S | H | r | W | Bpa4 | P | Da15o3pt | 2968 | 0.24 |
| 17 | Seq209 | DO3A10CM | Da48oa | Dnal2 | R | W | S | H | r | W | Bpa4 | p | Da15o3pt | 3100 | 0.14 |

BRU NO. 2727 in Table 13 immediately above corresponds to SEQ ID NO: 191 in the Sequence Listing.

An appealing observation for the potency of the LHRH-II analogs with a diamino acid such as Dap or Lys as a linker was that not much deterioration in binding was noted which indicated the requirement of the free amine of the linker at a critical distance from the peptide backbone for better binding. Keeping this in mind, L-4,8-diaminooctanoic acid (Da48oa) was introduced as a linker between AA¹ and DO3A10CM in the potent analog BRU-2968 (EC₅₀=0.24 μM) which has an oxyalkylamine (1,5-diamino-3-oxapentane, Da15o3pt) at the C-terminus. This resulted in BRU-3100, an agonist with high in vitro potency, EC₅₀=0.14 μM.

In Table 14 are provided the names and structures of amines and unusual/unnatural amino acids used in the synthesis of N-chelated analogs of LHRH-II.

TABLE 14
Names, Structures and Abbreviations of Amines and
Unnatural Amino Acids

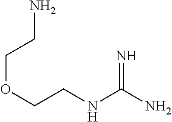

Amino[2-(2-aminoethoxy)ethyl]-carboxamidine
A1guao3pt

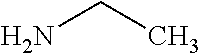

Aminoethane
Ae

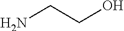

Aminoethanol
Aeh2

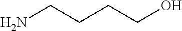

4-Aminobutanol
Abt1h4

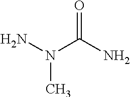

2,3-Diaza-2-methylpropionamide
Az23m2po

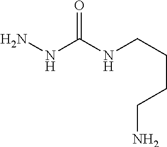

N-Amino[(4-aminobutyl)amino]-carboxamide
Az23po-Dabt14

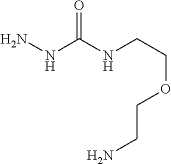

N-Amino{[2-(2-aminoethoxy)ethyl]-Amino}carboxamide
Az23po-Da15o3pt

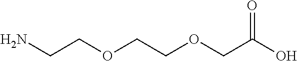

8-Amino-3,6-dioxaoctanoic acid
Adoa

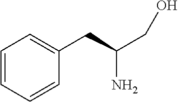

(2R)-2-Amino-3-phenylpropan-1-o [symbol]
Phenol

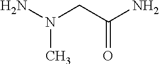

Amino-N-[(aminomethylamino)-methyl]amide
Az34mbuo-NH2

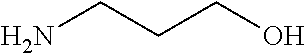

3-Amino-1-propanol
Aprp1h3

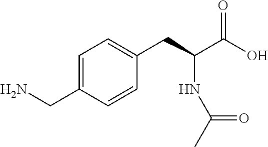

Alpha-N-Acety1-4-aminomethyl-L-phenylalanine
Ac-Amfe4

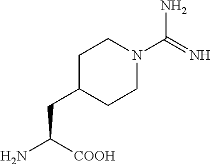

L-1-Amidino-4-piperidylalanine
Ampa4

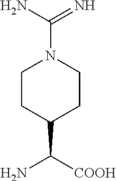

L-(1-Amidino-4-piperidyl)-glycine
Ampg4

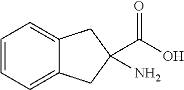

2-Aminoindane-carboxylic acid
Aic2

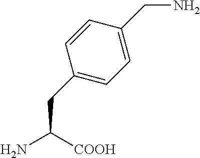

4-Aminomethyl-L-phenylalanine
Amfe4

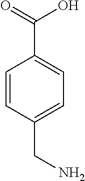

4-Aminomethy-benzoic acid
Amb4

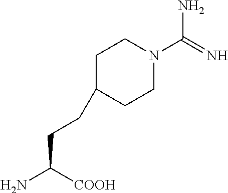

L-2-Amino-4-[4-(1-amidino)-piperidyl]-butyric acid
Ampha4

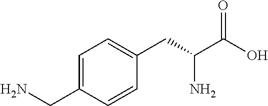

D-4-Aminomethyl-phenylalanine
Damfe4

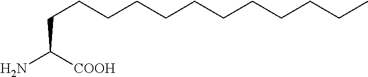

L-2-Amino-tetradecanoic acid
Atdc2

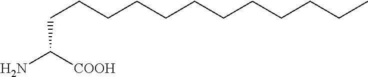

D-2-Amino-tetradecanoic acid
Datdc2

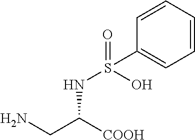

L-3-Amino,2-phenylsulfonamidopropionic acid
Apsp

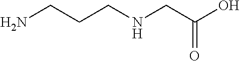

N-(3-Aminopropyl)-glycine
Apgly

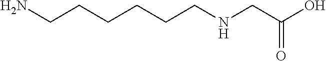

N-(6-Aminohexyl)-glycine
Ahgly

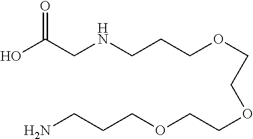

N-(13-Amino-4,7,10-trioxa-tridecyl)-glycine
Tdgly

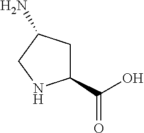

trans-4-Amino-L-proline
Ampt4

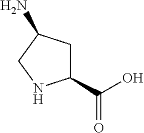

cis-4-Amino-L-proline
Ampc4

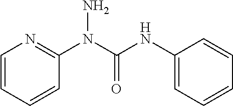

N-Amino(phenylamino)-N-(2-Pyridyl)-carboxamide
Az23(py2)2po-Ap

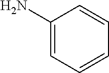

Aminophenyl
Ap

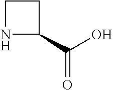

L-Azetidine-2-carboxylic acid
Aze

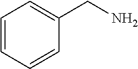

Aminobenzyl
Abn

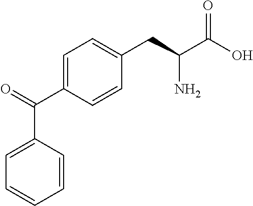

L-4-Benzoylphenylalanine
Bpa4

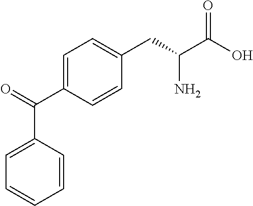

D-4-Benzoylphenylalanine
Dbpa4

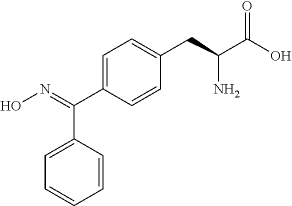

L-4-Benzoylphenylalanine-NO [symbol]
Bpa4(NOH)

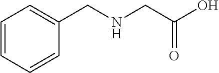

N-Benzylglycine
Bzgly

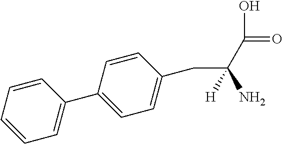

Biphenylalanine
Bip

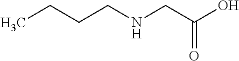

N-Butylglycine
Bugly

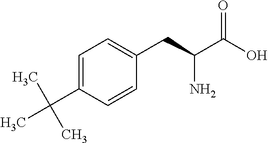

4-t-Butyl-L-phenylalanine
Tbufe4

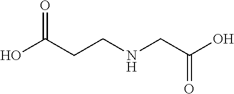

N-(2-Carboxyethyl)-glycine
Cegly

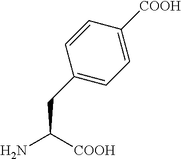

4-Carboxy-L-phenylalanine
Cafe4

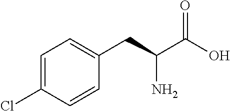

L-4-Chlorophenylalanine
Cfe4

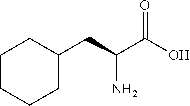

L-Cyclohexylalanine
Cha

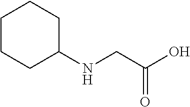

N-Cyclohexylglycine
Chgly

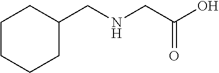

N-Cyclohexylmethyl-glycine
Chmgly

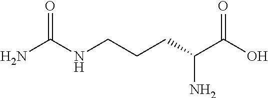

D-Citrulline
Dcit

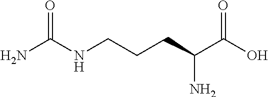

L-Citrulline
Cit

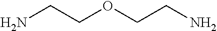

1,5-Diamino-3-oxapentane
Da15o3pt

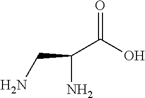

L-Diaminopropionic acid
Dap

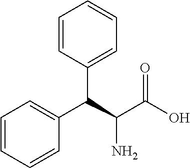

L-Diphenylalanine
Dip

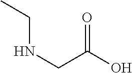

N-Ethylglycine
Etgly

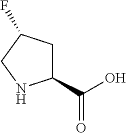

trans-4-Fluoro-L-proline
Flpt4

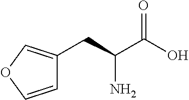

L-3-Furanylalanine
Fur3ala-

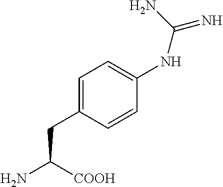

L-4-Guanidylphenyl-alanine
Gufe4

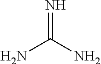

Guanidine
Gua

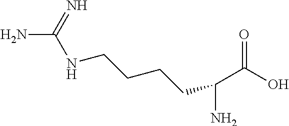

D-Homoarginine
Dharg

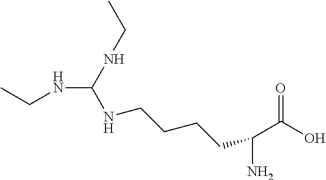

D-Homoarginine(diethyl)
Dharg(Et)₂

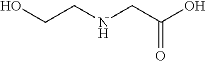

N-(2-Hydroxyethyl)-glycine
Hegly

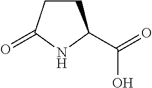

L-Pyroglutamic acid
pGlu

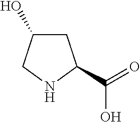

trans-4-hydroxy-L-proline
Hypt4

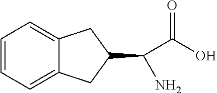

2-Indanyl-L-glycine
Ing2

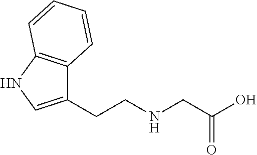

N-[2-(3-Indolyl)-ethyl]-glycine
Iegly

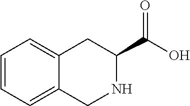

Isoquinolinine-L-3-carboxylic acid
Tic

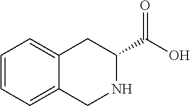

Isoquinolinine-D-3-carboxylic acid
Dtic

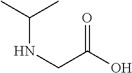

N-Isopropylglycine
Ipgly

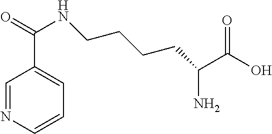

D-Lysine(Nicotinyl)
Dlys(Nic)

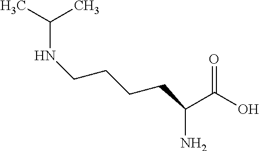

L-Lysine(i-Pr)
Lys(iPr)

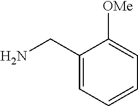

2-Methoxybenzylamine
Mo2abn

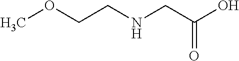

N-(2-Methoxyethyl)glycine
Mogly

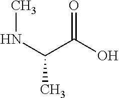

N-Methyl-L-alanine
Meala

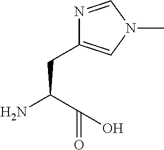

1-Methyl-L-histidine
His(1me)

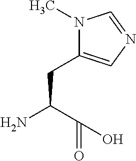

pi-Methyl-L-histidine
His(pime)

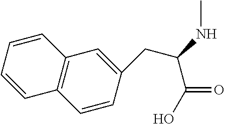

N-Methyl-2-Naphthyl-D-alanine
Mednal2

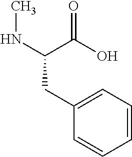

N-Methyl-L-phenylalanine
Mephe

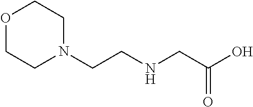

N-[2-(Morpholin-4-y1)-ethyl]-glycine
Mpgly

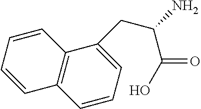

1-Naphthyl-L-alanine
Nal1

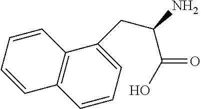

1-Naphthyl-D-alanine
Dnal1

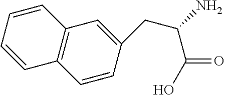

2-Naphthyl-L-alanine
Nal2

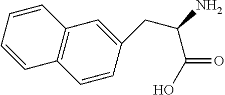

2-Naphthyl-D-alanine
Dnal2

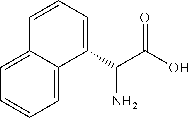

D-1-Naphthylglycine
Dnalgl

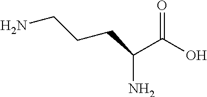

L-Ornithine
Orn

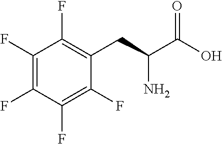

L-Pentafluorophenylalanine
F5fe

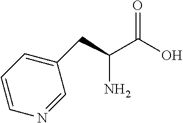

L-3-Pyridylalanine
Pa13

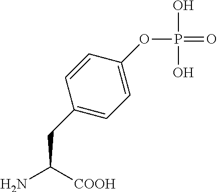

L-4-Phosphotyrosine
Pstr4

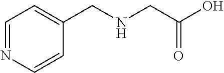

N-(4-Pyridylmethyl)-glycine
Prgly

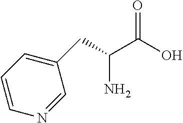

D-3-Pyridylalanine
Dpal3

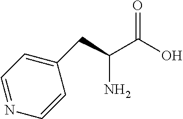

L-4-Pyridylalanine
Pa14

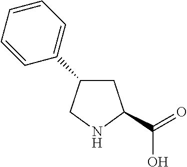

trans-4-Phenyl-L-proline
Ppt4

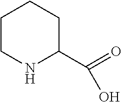

L-Pipecolic acid
(L-Homoproline)
Pip

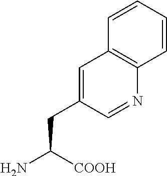

3-Quinolinyl-L-alanine
Qua3

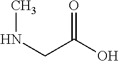

Sarcosine
Sar

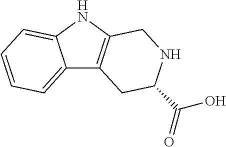

L-2,3,4,9-Tetrahydro-1H-beta-carboline-3-carboxylic acid
Tpi

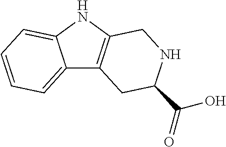

D-2,3,4,9-Tetrahydro-1H-beta-carboline-3-carboxylic acid
Dtpi

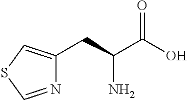

L-(4-Thiazolyl)-alanine
Tha

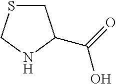

L-4-Thiaproline
Thz

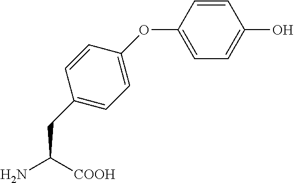

L-2-Thyronyl alanine
Thy

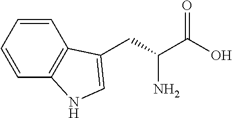

D-Tryptophan
Dtrp

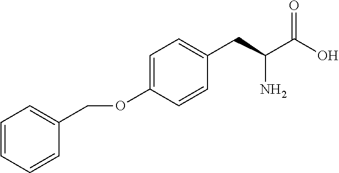

L-Tyrosine-O-benzyl ether
Tyr(Bzl)

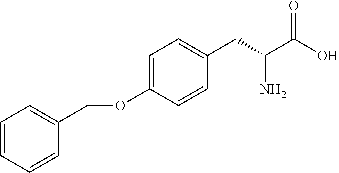

D-Tyrosine-O-benzyl ether
Dtyr(Bzl)

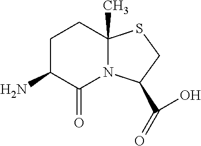

Hexahydro-5-oxo-6-amino-5H-thiazolo[3,2a]
pyridine-3-carboxylic acid
Btd

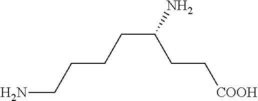

L-4,8-diaminooctanoic acid
Da48oa

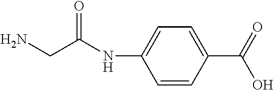

Glycyl-aminobenzoic acid
Gly-Abz4

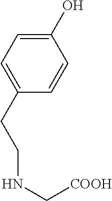

N-[2-(4-hydroxyphenyl)ethyl]-glycine)
Hpgly [symbol]
indicates data missing or illegible when filed

LHRH-II Analogs without Chelator

Based on the same principles described above for substitutions at various positions in the primary peptide sequence, a number of analogs not conjugated to DO3A10CM (or any other moiety) were prepared. It was observed that these principles for substitution applied in this context as well; a number of such peptides, in particular BRU-2441, -2734, -3007, -2439, -2839, -2803, -2821 and -2822, also exhibited increased binding affinity under the same binding-assay conditions. The sequences of these peptides and other relevant data are seen in Table 26.

### LHRH-II Analogs Bearing a Detectable Label (e.g. the Chelator DO3A10CM) at the C-Terminus

The possibility of using LHRH-II analogs bearing a detectable label such as the chelator DO3A10CM at the C-terminus was also explored. Such compounds have potential diagnostic and/or therapeutic applications. It was decided to incorporate into such C-terminus-conjugated analogs positional changes similar to those made in the LHRH-II analogs containing the DO3A10CM chelator on the N-terminus (nearly 200 in total) that were synthesized and screened as described above. BRU-2441 and BRU-2813 emerged as the lead structures from the initial screening assays. Their structures are shown below. The general structure of the compounds prepared in this series is also shown below.

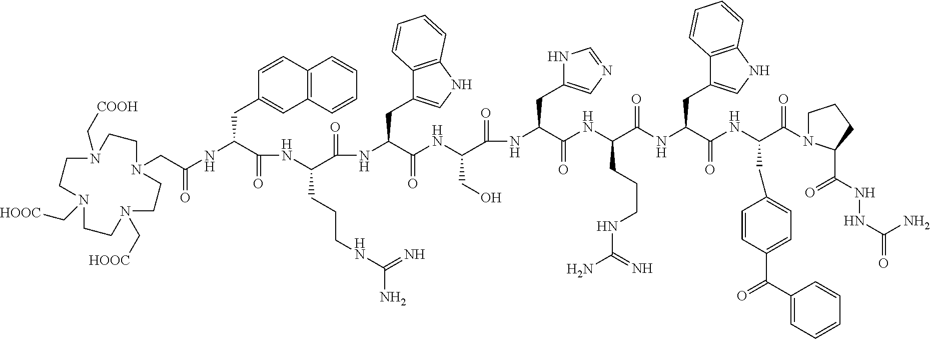

### BRU-2813

### DO3A10CM-Dnal2-Arg-Trp-Ser-His-Darg-Trp-Bpa-Pro-azaGly-NH₂

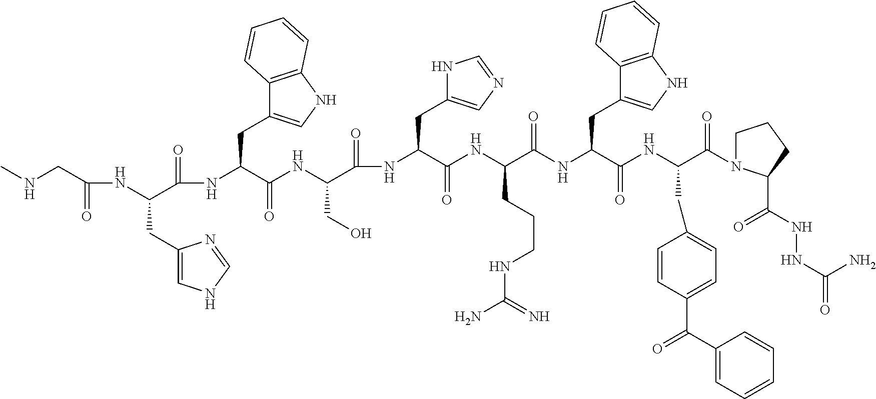

### BRU-2441

### Sar-His-Trp-Ser-His-Darg-Trp-Bpa-Pro-azaGly-NH₂

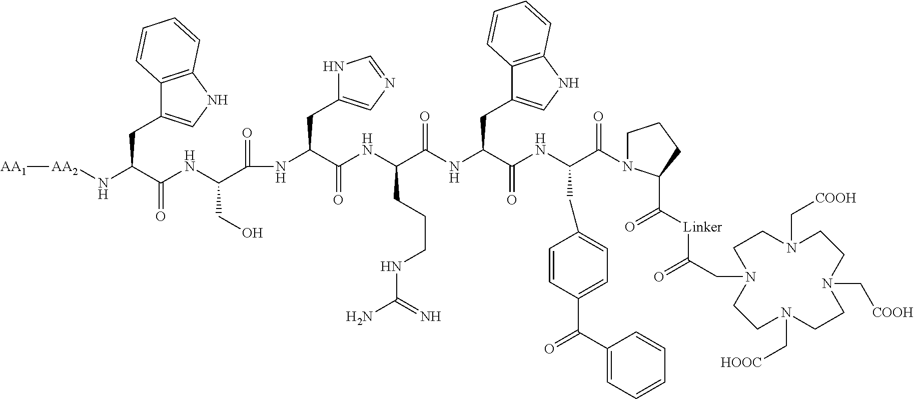

### General Structure of the C-terminus-Derivatized Peptides Prepared

### AA¹=Amino acid 1/Dnal2/Sar

### AA²=Amino acid 2/His or Arg

### (SEQ ID NO: 192)

The disclosure following outlines efforts to fine-tune the structure of BRU-2813/2441 by placing the DO3A10CM chelator at the C-terminus to increase the potency of the LHRH-II analogs.

The analog peptides bearing chelator at the C-terminus were synthesized as set forth below. Assessment of the binding affinities of the synthesized peptides was performed via the competitive and direct binding assays described previously herein.

### Preparation of LHRH Derivatives Bearing a Chelator at the C-Terminus

### Analytical HPLC Conditions:

Column: X-Terra® MS C₁₈ (Waters Corp.), RP; Particle size: 5.0μ; Solvent A: Water with 0.1% TFA (v/v) and Solvent B: Acetonitrile with 0.1% TFA (v/v); Elution rate; 3.0 mL/min; Detection at 220 nm.

Method (i): Initial conditions: 20% B; Gradient 20-60% B over 10 min
Method (ii): Initial conditions: 15% B; Gradient: 15-45% B over 15 min
Method (iii): Initial conditions: 20% B; Gradient: 20-60% B over 15 min

### Preparative HPLC Conditions:

Column-Atlantis® (Waters Corp.) C₁₈, RP; Particle size: 10.0μ; Solvent A: Water with 0.1% TFA (v/v) and Solvent B: Acetonitrile with 0.1% TFA (v/v); Elution Rate: 100.0 mL/min; Detection at 220 nm; Initial conditions: 10.0% B; Gradient: 10-20% B over 10 min and 20-70% B over 100 min. Approximately 10.0 mL fractions were collected and the fractions with the required peptide and purity >95% were pooled and freeze dried to yield the products as colorless fluffy TFA salts.

### A) Solid Phase Peptide Synthesis

Fully protected Boc-Dnal2/Boc-Sar-Arg(Pmc)-Trp(Boc)-Ser(Bu)-His(Trt)-Darg(Pmc/Pbf)-Trp(Boc)-Bpa-Pro-OH and Boc-Dnal2/Boc-Sar-Arg(Pmc)-Trp(Boc)-Ser(Bu)-His(Trt)-Darg(Pmc/Pbf)-Trp(Boc)-Bpa-Pro-Gly-OH were prepared on either Fmoc-Pro-NovaSyn-TGT resin® (0.22 mmol/g) and/or Fmoc-Gly-NovaSyn-TGT resin® (0.22 mmol/g) using an ABI-433A automated peptide synthesizer (Applied Biosystems, Foster City, Calif.). The peptides were assembled on resin using the FastMoc™ protocol, usually on a 0.25 mmol scale. After chain elongation was completed, the resin was washed with DCM (4×). The resin was then transferred to a manual peptide synthesizer vessel and shaken with 70:30 DCM/HFIPA for 1 h. The resin was drained and washed with 2×10 mL of DCM and the combined filtrates were concentrated under reduced pressure to yield, as colorless foam, the fully protected peptide sequence with a free carboxylic acid group at the C-terminus

### B) Manual Removal of the Fmoc Protecting Group

The resin containing the Fmoc-protected amino acid was treated with 20% piperidine in DMF (v/v, 15 mL/g resin) for 10 min. The solution was drained from the resin. This procedure was repeated once more followed by washing the resin with DMF (4×).

### C) Manual Deprotection of Peptides by Solution Phase Synthesis

A 20.0 mL portion of cleavage cocktail (95:4.5:0.5—TFA:Water:TIPS) was added to the final crude peptide in a round bottom flask and stirred for 4 h at ambient temperature. Volatiles were removed under reduced pressure at RT to give a paste which was triturated with 20.0 mL of absolute ether. The resulting solid was collected by filtration and washed with 2×10 mL of dry ether and then purified by preparative HPLC.

D) Synthesis of Peptides with C-terminus Amide Groups or Functionalized Amide Groups

About 0.08 mmol (0.2 g from procedure A) of the fully protected peptide sequence with a free carboxyl at the C-terminus was dissolved in 200 μL of DMF and treated sequentially with 0.81 mmol of N-hydroxysuccinimide and 1.0 mmol of DIC and stirred at ambient temp for 4 h. The resulting crude NHS ester was then added dropwise to a solution of a diamine (2.0 mmol) in 200 μL of DMF over a period of 10.0 min with vigorous stirring. After nearly 16 h, the reaction mixture was diluted with 100.0 mL of water and the aqueous solution was extracted with 3×50 mL of EtOAc. The combined organic layers were washed with water (2×50 mL), saturated sodium carbonate (2×50 mL), water (2×50 mL) and finally with saturated NaCl solution (1×50 mL) and dried (Na₂SO₄). The solution was filtered from the drying agent, concentrated to a paste under reduced pressure and the crude peptide was dried in vacuo for 1 h. The crude amine was acylated with DO3A10CM as described in procedure H below.

### E) Synthesis of Peptide Sequence on Diamine Bearing Trityl Resin

The first amino acid to be loaded (1.0 mmol) was dissolved in DMF (5.0 mL), activated with HOBt.H₂O (1.0 mmol), HBTU (1.0 mmol) and DIEA (2.2 mmol), and stirred for 10 min. This solution was transferred to the requisite diamine-bearing trityl resin (0.25 mmol) in a manual peptide synthesis vessel and this was agitated for 12 h. The vessel was drained and washed with 3×15 mL of DMF. The above resin was then transferred to a reaction vessel on the ABI-433A peptide synthesizer and the rest of the sequence was appended using ABI FasMoc™ protocols. After the appendage of the last amino acid, the resin was transferred to a manual peptide synthesis vessel and treated with 15.0 mL of DCM/TFA/TIPS (95:5:0.1) over 1 h to effect cleavage of the peptide from the resin. The vessel was drained and the resin was washed with DCM (3×10 mL). All the washings were combined and neutralized with 100 mL of saturated sodium carbonate solution. The organic layer was separated and washed with saturated sodium carbonate (2×25 mL), water (2×50 mL) and dried (Na₂SO₄). Removal of the solvent under reduced pressure yielded the crude C-terminus amine bearing peptide as colorless foam. The product was dried in vacuo (2 h) and was used in the final manual coupling of DO3A10CM using procedure detailed below (Refer to Procedure H below).

F) Synthesis of Modified aza-Gly on Resin

Diamine-bearing trityl resin and/or free-amine-bearing PAL-PEG-PS resin (Fmoc removed) (0.25 mmol) was suspended in 10 mL of anhydrous THF and CDI (2.5 mmol) was added to the resin in a manual peptide synthesis vessel and followed by agitation for 4 h. The vessel was drained and the resin was treated with a 1% solution of the requisite hydrazine derivative in DMF (3×20 mL). The resin was again washed with 3×20 mL of DMF and agitated with 20.0 mmol of the corresponding hydrazine in 20.0 mL of DMF for 12 h. The vessel was drained and the resin was washed with 3×20 mL of DMF and submitted to the next coupling.

The required amino acid (1.0 mmol) was dissolved in 10.0 mL of anhydrous THF, cooled to −10° C. and kept under nitrogen atmosphere. Isobutylchloroformate (1.0 mmol) was added via syringe with stirring, followed by NMM (1.01 mmol). The reaction mixture was allowed to come to 0° C. and stirred for 30 min. This activated acid was then transferred to the mixed urea on the resin and agitated for 12 h. The resin was then drained and washed with 1:1 DMF/MeOH (3×20 mL) and then with DMF (3×20 mL). The resulting peptide segment on the resin was taken through the rest of the sequence-building process on the ABI automated synthesizer. After the addition of the last amino acid, the resin from the ABI-433A synthesizer was transferred to a manual peptide synthesis vessel and shaken with 95:5:0.1-DCM:TFA:TIPS (20 mL) for 1 h. The resin was filtered and washed with 3×10 mL of DCM and the combined filtrates were neutralized with saturated sodium carbonate (100 mL). The organic layer was separated and washed with saturated sodium carbonate (2×25 mL), water (2×50 mL) and dried (Na₂SO₄). Removal of the solvent under reduced pressure yielded the crude C-terminus amine-bearing peptide as colorless foam. The product was dried in vacuo (2 h) and was used in the final manual coupling of DO3A10CM using the procedure detailed in Section H below.

G) Loading of Diamines onto Trityl Chloride Resin

Trityl chloride resin (0.25 mmol) was pre-swelled for 15 min with 1:1-DMF: DCM (10.0 mL) in a peptide synthesis vessel. The vessel was drained and a solution of 1.0 mmol of the required diamine in 1:1-DMF:DCM (5.0 mL) was added to the resin followed by agitation for 12 h. The vessel was drained under a positive pressure of nitrogen and the resin was washed with anhydrous pyridine (3×15 mL), and ether (3×20 mL). The amine-loaded resin was dried under high vacuum (2 h, <0.1 mm). The loading was assumed to be 100%.

The first amino acid (1.0 mmol) and HOBt.H₂O (1.0 mmol) and PyBOP (0.95 mmol) were dissolved in DMF (5.0 mL) and DIEA (2.0 mmol) was added and the mixture was shaken for 5 min at ambient temp. The solution of the activated amino acid was transferred to the amine-bearing trityl resin and the vessel was agitated for 12 h. The resin was drained under a positive pressure of nitrogen and washed with DMF (3×15 mL). The resin was transferred to a reaction vessel on the ABI-433A peptide synthesizer and the chain was elongated using the FastMoc® protocol. After chain elongation, the resin was washed with 4×20 mL of DCM and transferred back to a manual peptide synthesis vessel. The amine attached to the resin was released and worked up as detailed in procedure E and manually acylated in solution with DO3A10CM using procedure H below.

H) General Procedure for Introduction of DO3A10CM onto the Peptide Chain

DO3A10CM (tris-t-Bu) ester (4.0 equiv.), HOBt.H₂O (4.0 equiv.) and HBTU (4.0 equiv.) were dissolved in 5.0 mL of DMF and DIEA (8.8 equiv.) was added followed by stirring at room temperature for 10 min. This activated acid in DMF was transferred to the crude amine in a RB flask. An additional 1.0 mL of DMF was used to transfer the remaining activated acid to the amine and the reaction mixture was stirred for 20 h at ambient temperature. The solution was diluted with 100.0 mL of saturated sodium carbonate and extracted with 3×50 mL of EtOAc. The combined extracts were washed with 2×50 mL of saturated sodium carbonate, water (2×50 mL), saturated sodium chloride (1×50 mL) and dried (Na₂SO₄). Removal of the solvent under reduced pressure yielded the crude peptide as an off-white foam. The crude peptide was deprotected using procedure C and purified by preparative HPLC.

### I) Synthesis of (S)-2-Aminomethylpyrrolidine:

This diamine was prepared as reported⁶¹ and loaded on to trityl chloride resin. The first amino acid was added using procedure G.

### J) Preparation of 2,6-Bisaminomethylpyridine:

Prepared as described in the literature⁶² and loaded on to trityl chloride resin. The first amino acid was appended to the resin manually.

### Peptide 1 (BRU-2990) (SEQ ID NO: 193):

Yield: 2.3 mg (0.33%); Methods of preparation—A, B, C, D, H: t_{R}—3.49 min (i); M. S.—API-ES positive ion mode: [M+2TFA+2Na]/2: 1130.4; [M+2TFA+H]/2: 1108.4; [M+2TFA+2H]/4: 554.8

### Peptide 2 (BRU-2991):

Yield: 10.3 mg (1.06%); Methods of preparation—A, B, C, D, H: t_{R}—3.64 min (i); M. S.—API-ES positive ion mode: [M+2H]/2: 973.4; [M+3H]/3: 649.2

### Peptide 3 (BRU-2992):

Yield: 7.0 mg (1.05%); Methods of preparation—A, B, C, D, H: t_{R}—4.31 min (i); M. S.—API-ES positive ion mode: [M+2H]/2: 994.4; [M+3H]/3: 663.2

### Peptide 4 (BRU-3039):

Yield: 38.5 mg (6.3%); Methods of preparation—A, B, C, D, H: t_{R}—2.76 min (i); M. S.—API-ES positive ion mode: [M+2Na]/2: 932.2; [M+Na+H]: 921.2; [M+2H]/2: 910.2; [M+3H]/3: 607.2; [M+4H]/4: 456.6

### Peptide 5 (BRU-3041):

Yield: 22.7 mg (3.9%); Methods of preparation—A, B, C, D, H: t_{R}—2.89 min (i); M. S.—API-ES positive ion mode: [M+2H+TFA]: 959.9; [M+H+K]/2: 921.2; [M+H+Na]/2: 913.4; [M+2H]/2: 902.4; [M+Na+3H]:/3: 640.2; [M+2H+K]/3: 614.4; [M+3H]/3: 601.8; [M+4H]/4: 451.6;

### Peptide 6 (BRU-3042):

Yield: 14.2 mg (2.2%); Methods of preparation—A, B, C, D, H: t_{R}—3.76 min (i); M. S.—API-ES positive ion mode: [M+2H]/2: 965.4; [M+3H]/3: 643.8; [M+4H]/4: 483.2

### Peptide 7 (BRU-3043):

Yield: 21.5 mg (1.2%); Methods of preparation—A, B, C, E, H: t_{R}—6.18 min (ii); M. S.—API-ES positive ion: [M+2H+TFA]: 989.8; [M+2H]/2: 932.4; [M+3H+TFA]: 660.2;[M+3H]/3: 621.8; [M+4H]/4: 466.6

### Peptide 8 (BRU-3044):

Yield: 61.0 mg (9.6%); Methods of preparation—A, B, C, D, H: t_{R}—5.15 min ii); M. S.—API-ES positive ion: [M+2H]/2: 958.4; [M+3H]/3: 639.2; [M+4H]/4: 479.8; [M+TFA+2Na]/4: 517.2

### Peptide 9 (BRU-3045):

Yield: 25.5 mg (0.83%); Methods of preparation—A, B, C, D, H: t_{R}—5.09 min (ii); M. S.—API-ES positive ion mode: [M+3Na−H]/2: 963.8; [M+2Na]/2: 952.8; [M+Na+H]/2: 941.8; [M+2H]/2: 930.8; [M+3H]/3: 620.8; [M+4H]/4: 466.0

### Peptide 10 (BRU-3073) (SEQ ID NO: 194):

Yield: 9.0 mg (0.5%); Methods of preparation—A, B, C, F, H: t_{R}—6.46 min (ii); M. S.—API-ES positive ion mode: [M+2H]/2: 987.8; [M+3H]/3: 658.8; [M+4H]/4: 494.4

### Peptide 11 (BRU-3074) (SEQ ID NO: 8):

Yield: 19.0 mg (3.3%); Methods of preparation—A, B, C, D, F, H: t_{R}—5.07 min (ii); M. S.—API-ES positive ion mode: [M+H]: 1728.6; [M+2H]/2: 864.4; [M+3H]/3: 576.8

### Peptide 12 (BRU-3076) (SEQ ID NO: 8):

Yield: 20.0 mg (3.3%); Methods of preparation—A, B, C, D, H: t_{R}—5.64 min (ii); M. S.—API-ES positive ion mode: [M+3TFA+2Na]/2: 1110.0; [M+2H]/2: 916.8; [M+3H]/3: 611.4; [M+3TFA+3Na]/3: 747.6

### Peptide 13 (BRU-3079) (SEQ ID NO: 195):

Yield: 29.0 mg (5.1%); Methods of preparation—A, B, C, D, H: t_{R}—5.06 min (ii); M. S.—API-ES positive ion mode: [M+H]: 1756.8; [M+2H]/2: 878.4; [M+3H]/3: 586.2

### Peptide 14 (BRU-3080):

Yield: 48.5 mg (7.8%); Methods of preparation—A, B, C, D, H: t_{R}—5.57 min (ii); M. S.—API-ES positive ion mode: [M+H]: 1852.8; [M+Na+H]: 937.4; [M+2H]/2: 926.8; [M+3H]/3: 618.2; [M+4H]/4: 464.0

### Peptide 15 (BRU-3085):

Yield: 58.0 mg (9.6%); Methods of preparation—A, B, C, D, H: t_{R}—3.25 min (iii); M. S.—API-ES positive ion mode: [M+2H]/2: 909.4, [M+3H]/3: 606.4; [M+4H]/4: 455.2

### Peptide 16 (BRU-3086) (SEQ ID NO: 8):

Yield: 18.1 mg (0.9%); Methods of preparation—A, B, C, G, H: t_{R}—2.91 min (iii); M. S.—API-ES positive ion mode: [M+Na+H]/2: 941.8; [M+H]/2: 930.8; [M+3H]/3: 620.8; [M+4H]/4: 466.0.

### Peptide 17 (BRU-3102):

Yield: 72.0 mg (3.9%); Methods of preparation—A, B, C, G, H: t_{R}—3.12 min (i); M. S.—API-ES positive ion mode: [M+H]: 1829.8; [M+Na+H]/2: 926.4; [M+2H]/2: 915.4; [M+3TFA-3H]/3: 722.0; [M+3H]/3: 610.6; [M+2K+Na+H]/4: 481.8; [M+4H]/4: 458.2

### Peptide 18 (BRU-3103):

Yield: 15.0 mg (2.4%); Methods of preparation—A, B, C, D, H: t_{R}—2.83 min (i); M. S.—API-ES positive ion mode: M+H]: 1774.8; [M+H+Na]/2: 899.2; [M+2H]/2: 888.4; [M+2H+Na]/3: 599.8; [M+3H]/3: 592.6

### Peptide 19 (BRU-3117):

Yield: 7.7 mg (0.4%); Methods of preparation—A, B, C, G, H: t_{R}—2.89 min (i); M. S.—API-ES positive ion mode: [M+2H]/2: 859.4, [M+3H]/3: 573.4, [M+4H]/4: 430.4, [M+Na+H]/2: 870.8

At the outset, the analogs described herein seemed amenable to synthesis by straightforward peptide synthesis methods. However during the course of the work it became clear that standard peptide synthesis protocols could not be used for all the steps needed to complete the synthesis. Both solid phase and solution phase synthetic techniques and/or improvements to the existing synthetic protocols were required. The preparation of these peptides involved three different procedures.

In procedure 1 (as represented by peptide 1, Scheme 3), amino acid chain AA₁-AA₉/AA₁₀ was prepared using solid phase synthesis on an automated synthesizer (ABI, Applied Biosystems, Inc.) and the fully protected peptide was cleaved from the resin to furnish the chain with a free carboxylic acid at the C-terminus Amidation of the acid with excess diamine in solution resulted in a free amino group at the C-terminus as the major component. Without further purification the crude amine was acylated with DO3A10CM (tris-t-butyl) ester. Subsequent deprotection and purification provided the expected product as TFA salt. Our initial attempts to prepare these peptides on the solid phase starting from the appropriately loaded diamines on trityl resins either failed or resulted in a mixture of products from which isolation of the required products proved very cumbersome.

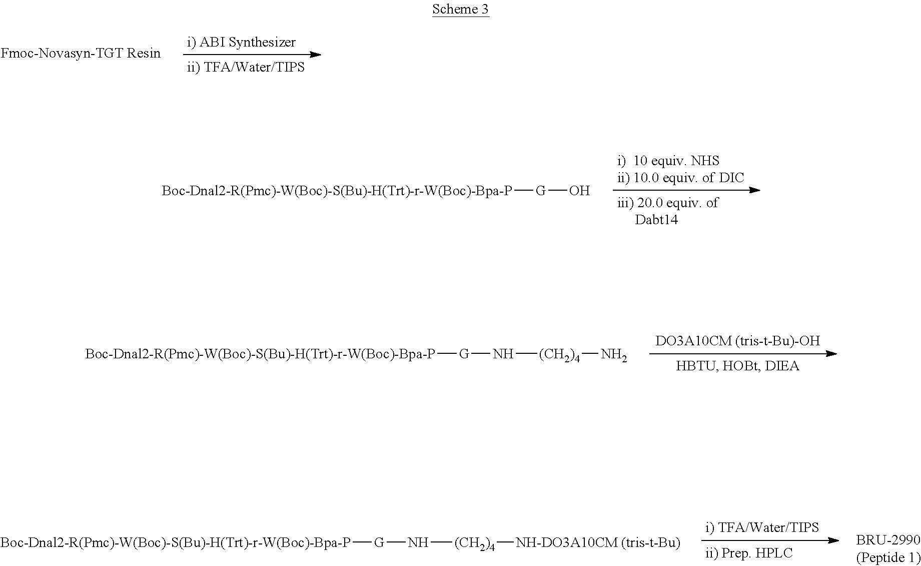

For peptide sequences that contained a proline amide of a secondary amine, the required secondary amines were initially loaded onto trityl chloride resin using standard procedures; the loading was assumed to be 100%. The nature of the secondary amine was exploited to good advantage, since the primary amino function was selectively alkylated by the trityl chloride⁶³ on the resin leaving the secondary amine for further manipulation. This method also avoided the selective protection and deprotection of the amines

However, introducing the first amino acid to the secondary amine did not work on the ABI-433A peptide synthesizer and needed to be carried out manually for longer reaction time to force the reaction. After the manual addition of the first amino acid, the rest of the sequence was added on the ABI-433A automated peptide synthesizer. The fully protected peptide was cleaved from the resin with 5% TFA in DCM and the resulting amine was acylated with DO3A10CM. The above method is illustrated in Scheme 4 by the synthesis of peptide 9.

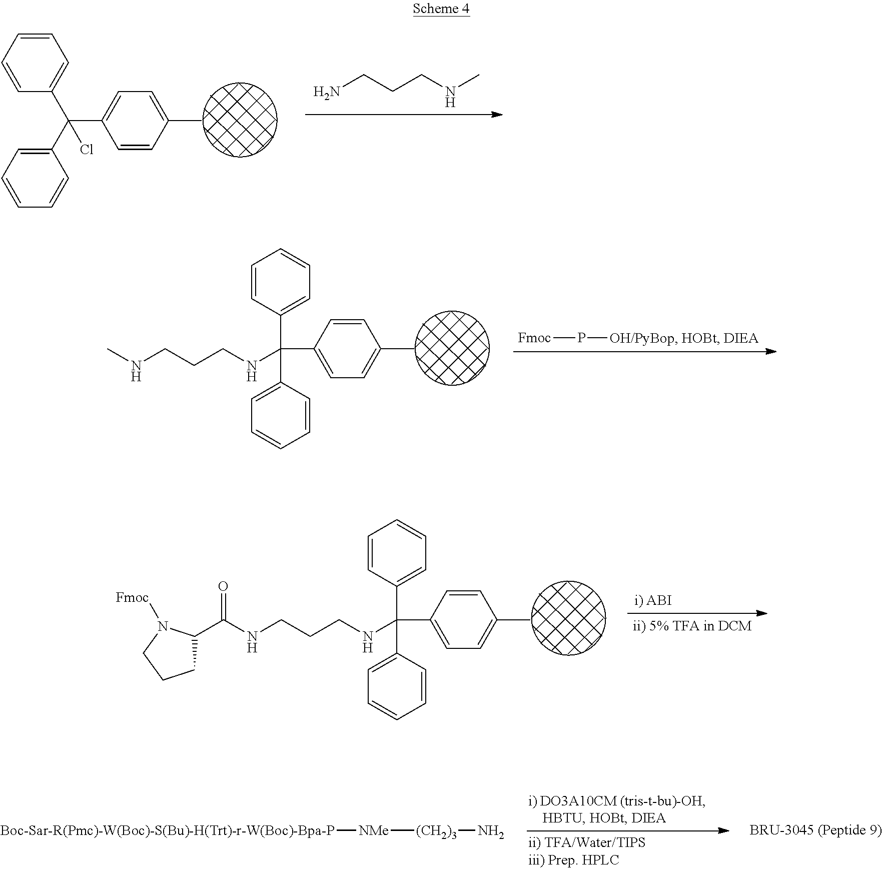

A third method involved the construction of a substituted semicarbazide on the resin. Attempted preparation of the required semicarbazide, represented by example (Table 15, peptide 10), was started from the corresponding diamine-bearing trityl resin. The amine on the resin was sequentially treated with CDI followed by hydrazine to assemble the semicarbazide. However the acylation of this with the first amino acid repeatedly failed using known coupling agents and conditions (PyBop, HBTU, HATU etc).

To overcome this difficulty, the amino acid was activated with isobutylchloroformate and NMM to form the mixed anhydride and then added to the semicarbazide on the solid phase. The acylation was carried out for 12 h. The reaction proceeded as expected and the rest of the peptide chain was then built on the resin with the aid of an automated synthesizer. After the final amino acid was added, the peptide was cleaved from the resin and acylated with DO3A10CM, deprotected and purified to yield the required LHRH-II analog. This procedure is outlined in Scheme 5

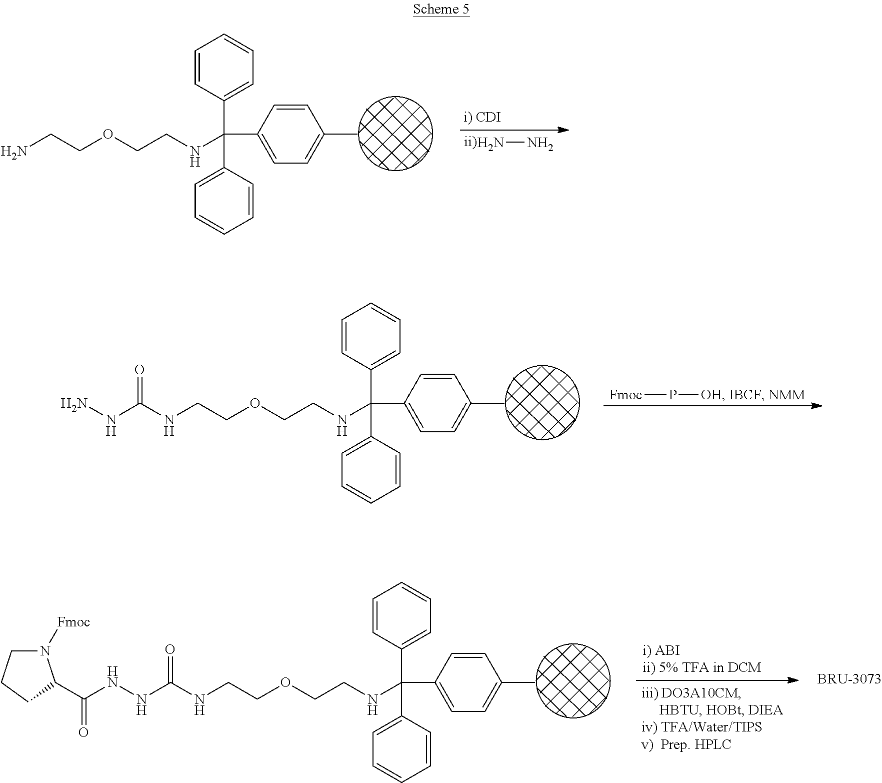

Table 15 below lists the 19 peptides prepared to probe the effects on the affinity of these molecules towards LHRH receptors when the reporter/chelator moiety was moved from the N-terminus to the C-terminus Competitive in vitro binding assays clearly indicated the influence of the linkers on binding.

TABLE 15
Summary of C-Terminus Chelator-Functionalized Peptides Synthesized

| | | | | Retention | | |
|---|---|---|---|---|---|---|
| | | | Synthetic | Time (min)/ | | |
| | BRU | | Methods | HPLC | | EC₅₀ |
| # | Number | Sequence | Used | Method | Mass | μM |
| 1 | 2990 | Dnal2-R-W-S-H-Darg-W-Bpa4-P-G- | A, B, C, D, H | 3.49, (i) | 1986 | 30.21 ± 8.28 |
| | | Dabt14-DO3A10CM | | | | |
| 2 | 2991 | Dnal2-R-W-S-H-Darg-W-Bpa4-P- | A, B, C, D, H | 3.64, (i) | 1945 | 0.21 ± 0.06 |
| | | Da15o3pt-DO3A10CM | | | | |
| 3 | 2992 | Ac-Dnal2-R-W-S-H-Darg-W-Bpa4- | A, B, C, D, H | 4.31, (i) | 1987 | 0.47 ± 0.02 |
| | | P-Da15o3pt-DO3A10CM | | | | |
| 4 | 3039 | Sar-R-W-S-H-Darg-W-Bpa4-P- | A, B, C, D, H | 2.76, (i) | 1819 | 0.28 ± 0.03 |
| | | Da15o3pt-DO3A10CM | | | | |
| 5 | 3041 | Sar-R-W-S-H-Darg-W-Bpa4-P- | A, B, C, D, H | 2.89, (i) | 1803 | 0.36 ± 0.02 |
| | | Dabt14-DO3A10CM | | | | |
| 6 | 3042 | Dnal2-R-W-S-H-Darg-W-Bpa4-P- | A, B, C, D, H | 3.76, (i) | 1929 | 0.2 ± 0.0 |
| | | Dabt14-DO3A10CM | | | | |
| 7 | 3043 | Sar-R-W-S-H-Darg-W-Bpa4-P- | A, B, C, E, H | 6.18, (ii) | 1863 | 0.33 ± 0.18 |
| | | Da18o36oc-DO3A10CM | | | | |
| 8 | 3044 | Sar-R-W-S-H-Darg-W-Bpa4-P- | A, B, C, D, H | 5.15, (ii) | 1915 | 0.24 ± 0.08 |
| | | bap14p-DO3A10CM | | | | |
| 9 | 3045 | Sar-R-W-S-H-Darg-W-Bpa4-P- | A, B, C, D, H | 5.09, (ii) | 1860 | 0.21 ± 0.08 |
| | | Maz4dahp17-DO3A10CM | | | | |
| 10 | 3073 | Sar-R-W-S-H-Darg-W-Bpa4-P-P- | A, B, C, F, H | 6.46, (ii) | 1974 | 0.84 ± 0.11 |
| | | Az23po-Da15o3pt-DO3A10CM | | | | |
| 11 | 3074 | Sar-R-W-S-H-Darg-W-Bpa4-P- | A, B, C, D, F, H | 5.07, (ii) | 1728 | 1.48 ± 1.03 |
| | | NHNH-DO3A10CM | | | | |
| 12 | 3076 | Sar-R-W-S-H-Darg-W-Bpa4-P- | A, B, C, D, H | 5.64, (ii) | 1832 | 0.75 ± 0.04 |
| | | Daprp13-DO3A10CM | | | | |
| 13 | 3079 | Sar-H-W-S-H-Darg-W-Bpa4-P-Dae- | A, B, C, D, H | 5.06, (ii) | 1756 | 0.59 ± 0.26 |
| | | DO3A10CM | | | | |
| 14 | 3080 | Sar-R-W-S-H-Darg-W-Bpa4-P- | A, B, C, D, H | 5.57, (ii) | 1852 | 0.23 ± 0.1 |
| | | Bampy26-DO3A10CM | | | | |
| 15 | 3085 | Sar-R-W-S-H-Darg-W-Bpa4-P- | A, B, C, D, H | 3.25, (iii) | 1817 | 0.4 ± 0.25 |
| | | Dapt15-DO3A10CM | | | | |
| 16 | 3086 | Sar-R-W-S-H-Darg-W-Bpa4-P- | A, B, C, G, H | 2.91, (iii) | 1860 | 1.02 ± 0.0 |
| | | M1daprp13-DO3A10CM | | | | |
| 17 | 3102 | Sar-R-W-S-H-Darg-W-Bpa4-P- | A, B, C, G, H | 3.12, (i) | 1829 | 0.2 ± 0.07 |
| | | Ampip2-DO3A10CM | | | | |
| 18 | 3103 | Sar-R-W-S-H-Darg-W-Bpa4-P-Dae- | A, B, C, D, H | 2.83, (i) | 1775 | 0.17 ± 0.07 |
| | | DO3A10CM | | | | |
| 19 | 3117 | Sar-R-W-S-H-Darg-W-Bpa4- | A, B, C, G, H | 2.89, (i) | 1718 | 0.34 ± 0.0 |
| | | Am2prd-DO3A10CM | | | | |

Most of the compounds synthesized had EC₅₀ values that were less than or equal to 1 μM when screened in a competition assay on human ovarian cancer (EFO-27) cells when competed with [¹²⁵I-Tyr⁸]BRU-2477. Peptides 2, 6, 9, 17, and 18 showed EC₅₀ values of less than or equal to 0.2 μM whereas 3, 4, 5, 7, 8, 14 and 15 showed values between 0.23-0.5 μM. Peptides 10, 12, 13 and 16 exhibited values between 0.59-1.0 μM and 11 had a value of about 1.5 μM.

One surprising exception was peptide 1 whose EC₅₀ value was >30.0 μM, suggesting that the folding of the amino acid chain and the hydrogen bonding nature of the linker might play a role in its ability to reach the receptor binding pocket site. It became apparent that the nature of the linker and length between Pro⁹ and the reporter play a crucial role in the affinity of these peptides towards the LHRH-II receptor. Peptide 1 clearly supports the above observation. Sequences 11 and 16 which contain a rigid linker (—NHNH—) and a flexible linker which could induce rigidity due to a tertiary amide bond (—NCH₃—CH₂—CH₂—CH₂—NH), reduced the binding by almost 5-6 fold when compared to peptide 18 (Table 15) with a short flexible linker (—NH—CH₂—CH₂—NH—). It is also conceivable that in these two molecules, the hydrogen bonding abilities of the linkers involved might somehow alter the folding of the peptide chain, thereby decreasing their ability to bind to the receptors. This notion is further supported by the values observed with sequence 10, where the aza-gly is modified to accommodate more substitution. However, flexible linkers between Pro⁹ and the reporter seem to preserve the binding, as noted with the rest of the sequences.

In Table 16 are set out various diamino linkers and unusual amino acids that are components of the C-terminus-chelated LHRH-II analogs prepared.

TABLE 16
Linkers and Unusual Amino Acids Used

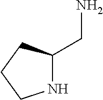

Am2prd
{(2S)-Pyrrolidine-2-yl}methylamine

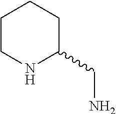

Ampip2
(±)-2-Aminomethylpiperidine

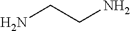

Dae
1,2-Diaminoethane

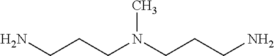

Maz4dahp17
Bis(3-aminopropyl)methylamine

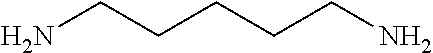

Dapt15
1,5-Diaminopentane

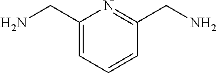

Bampy26
2,6-Bisaminomethyl-pyridine

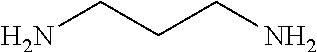

Daprp13
1,3-Diaminopropane

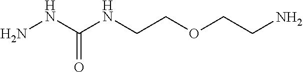

Az23po-Da15o3pt [symbol]
-Amino[(4-aminobutyl)amino [symbol] carboxamide

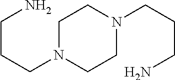

Bap14p
1,4-Bis(3-aminopropyl)-piperazine

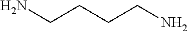

Dabt14
1,4-Diaminobutane

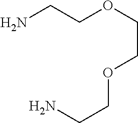

Da18o36oc
1,8-Diamino-3,6-dioxaoctane

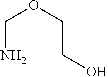

Da15o3pt
1,5-Diamino-3-oxapentane

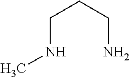

M1daprp13
Diaminopropane

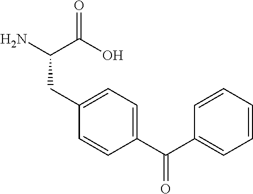

Bpa4
L-(4)-Benzoylphenylalanine

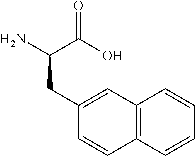

Dnal2
(D)-2-Naphthylalanine [symbol]
indicates data missing or illegible when filed

### Radiolabeling of LHRH Compounds Bearing a DO3A10CM Chelator at the C- or N-Terminus

The LHRH derivatives bearing a DO3A10CM ligand at the C- or N-terminus could be readily labeled with radioisotopes such as ¹⁷⁷Lutetium (Lu). They could also be derivatized with non-radioactive metals such as ¹⁷⁵Lu. The labeled compounds were used for evaluation in competition and direct cell binding studies with EFO-27 cells, and in in vivo and in vitro metabolism studies. Methods for the preparation and analysis of Lu-labeled compounds are described below.

### Preparation and Analysis of ¹⁷⁵Lu- and ¹⁷⁷Lu-Labeled Compounds for Cell Binding Studies

¹⁷⁷Lu is a mixed β and γ-emitter (t_{1/2}=6.71 days with a primary beta emission at 498 keV and gamma emissions at 208.4 and 112.9 keV), so has utility for both imaging and radiotherapy applications. ¹⁷⁵Lu is the most abundant isotope in natural (non-radioactive) Lu. All manipulations involving radioactivity were carried out behind lead/Plexiglas shielding using appropriate radiological precautions. The water used in these studies was in-house reverse osmosis feed water processed through carbon and ion exchange resins. Acetonitrile (HPLC grade), trifluoroacetic acid (Burdick & Jackson), glacial acetic acid (Ultrex® II Ultrapure Reagent, J. T. Baker), Bacteriostatic 0.9% Sodium Chloride for Injection, USP (Abbott Laboratories), ASCOR L₅₀₀® Ascorbic Acid Injection, USP (McGuff Pharmaceuticals, Inc.), Human serum albumin (HSA, Cat. No. A1653, Sigma), sodium acetate (NaOAc, 99% minimum: EM Science) and L-selenomethionine (Sabinsa) were used as received. ¹⁷⁷Lutetium (III) chloride (¹⁷⁷LuCl₃) dissolved in 0.05 N HCl, was obtained from Missouri University Research Reactor (MURR), Columbia, Mo. A lutetium plasma standard solution (Lu₂O₃ in 5% HNO₃,10000 μg/mL) was obtained from Alfa Aesar (Ward Hill, Mass.). BRU-2756, BRU-2757, BRU-2666, BRU-2443, BRU-2624, BRU-2721, BRU-2613 (SEQ ID NO: 196), BRU-2440, BRU-2797, BRU-2644 (SEQ ID NO: 197), BRU-2741, BRU-2722, BRU-2767, BRU-2642, BRU-2696, BRU-2736, BRU-2792, BRU-2738, BRU-2725, BRU-2742, BRU-2810, BRU-2812 (SEQ ID NO: 198), BRU-2813, BRU-2823, BRU-2869, BRU-2894 and BRU-2896 (SEQ ID NO: 12) were prepared in house as described earlier. Dulbecco's phosphate-buffered saline (DPBS) containing 1 mM Ca²⁺ and 1 mM Mg²⁺, supplemented with BSA (0.2%), HEPES (20 mM), and Bacitracin (100 mg/L) was the binding buffer used to dilute the Lu-labeled product after metal incorporation.

HPLC analysis was performed using an Agilent Technologies 1100 Series HPLC equipped with a solvent degasser, quaternary pump, autosampler, column compartment, single wavelength detector, ChemStation LC-3D software, Revision A.09.01[1206], and a Beckman (Fullerton, Calif.) Model 170 Radioisotope detector. The following HPLC method was used: Gradient elution from 85% H₂O (0.1% TFA v:v)/15% CH₃CN (0.1% TFA v:v) to 60/40 in 60 min. Column. Zorbax Bonus-RP (4.6×250 mm; 5 μm, Agilent), Flow rate: 1 mL/min, Column temp: 30° C. For radiodetection of ¹⁷⁷Lu-LHRH complexes, a 15 μL (˜90 μCi) injection was used. For analysis of ¹⁷⁵Lu-LHRH complexes, incorporation of the Lu into the ligand was monitored by UV at 280 nm.

Alternatively, for the analyses of the Lu-complexes of LHRH II analogs and their metabolites, the following HPLC method was used. Column: C4-AP (YMC, BU30SO5-2546WT; S-5 μm, 30 nm), Solvents: A: H₂O/0.1% TFA (v:v); B: ACN/0.1% TFA (v:v), Flow rate: 1.5 mL/min, Column temperature: 37° C., Gradient: 2% B/98% A for 0-2 mM; to 15% B/85% A in 3 mM; to 35% B/65% A in 43 min; to 90% B/10% A in 44-48 min; back to 2% B/98% A in 50 min with a 10 min post run.

For the analyses of BRU-2477, BRU-3122, BRU-3123 and BRU-3124, the following HPLC method was used: Column: Bonus RP 4.6×250 mm, Solvents: A: H₂O/0.1% TFA (v:v); B: ACN/0.1% TFA (v:v), Flow rate: 1.5 mL/min, Column temperature: 37° C., Gradient: 100% A for 4 min, to 75% A/25% B in 40 min; to 10% A/90% B in 2 min; hold for 2 min; to 100% A in 1 minute with a 10 min post run.

### Preparation of ¹⁷⁵Lu Complexes for Cell Binding Studies

For cell binding studies, the desired final ¹⁷⁵Lu complex concentration was that used for the direct binding studies (30 μM). The complexes were synthesized at a concentration of 300 μM and then diluted 10-fold with the buffer used for the cell binding experiment after labeling. A stoichiometry of 1.2 ¹⁷⁵Lu: 1 Ligand was typically used, as this provided sufficient Lu to complex all the ligand. Excess free ¹⁷⁵Lu did not interfere in the cell binding assay.

¹⁷⁵Lu-labeled LHRH complexes were prepared as follows: An amount of ligand necessary to achieve a concentration of 300 μM in 0.15 mL was dissolved at the concentration of 1 μg/μL in 10% DMSO/90% 0.05 M NaOAc pH 4.8 (in saline). The ligand solution and an aliquot of ¹⁷⁵Lu plasma standard (1.2 equivalents) were mixed and sufficient 0.05 M NaOAc pH 4.8 (in saline) was added to bring the volume to 0.15 mL. The mixture was heated at 100° C. for 10 min., cooled for 2 min in a water bath, and 1.35 mL of binding buffer was added to dilute the final complex to a concentration of 30 μM.

General Synthesis of ¹⁷⁷Lu-Complexes For the synthesis of ¹⁷⁷Lu complexes, ¹⁷⁷LuCl₃ (3-5 μL, about 2.5 mCi) was added to a 450 μL insert inside a 2 mL Agilent vial. The radioactivity was measured in a Capintec and based on the specific activity, the mass of ^{176/177}Lu was calculated using the following formula: [(A°(μCi)/SA (mCi/μg))*exp(−0.69313*decay time (h)/t_{1/2} (h)*(SA(mCi/μg)/theoretical SA(mCi/ug))+(SA(mCi/μg)/theoretical SA(mCi/ug)*(1−SA (mCi/μg)/theoretical SA (mCi/μg)] Where A°=activity of the sample at calibration time (A°=A(μCi)/exp(−0.69313*decay time (h)/t_{1/2} (h), A=activity at the time of measurement, SA=specific activity of the lutetium at calibration time, decay time=time from the calibration time to the time of measurement, t_{1/2}=half life of ¹⁷⁷Lu (168 h) and the theoretical SA=110 Ci/mg.

The volume of ligand solution needed to provide a stoichiometry of 4:1 or 8:1 between LHRH ligand and lutetium was added to the insert vial. Sufficient 0.05 M NaOAc pH 4.8 containing 1 mg/mL L-selenomethionine (Se-Met) as a radiostabilizer was added to bring the ligand concentration to 60 μM. This solution was heated at 100° C. for 10 min, cooled for 2 min in a water bath and diluted to 400 μL with 9 Saline: 1 Ascor L500: 0.1% HSA.

As an example, 2.61 mCi of ¹⁷⁷LuCl₃ was used to prepare ¹⁷⁷Lu-BRU-2756. The specific activity of this ¹⁷⁷LuCl₃ lot was 24.52 Ci/mg at the time of labeling. The amount of ^{176/177}Lu used was calculated to be 0.125 μg as follows:

A°=2610 μCi/exp(−0.69313*47.5 h/160.8 h=3203 μCi

μg of ^{176/177}Lu={[(3203 μCi/24520 mCi/μg)*exp(−0.69313*47.5 h/160.8 h)*(24520 mCi/μg/110000 mCi/μg)]+(3203 μCi/24520 mCi/μg)*(1-24520 mCi/μg/110000(mCi/μg)}=0.125 μg

0.125 μg/176*1810.02(mw BRU-2756)*4 eq=5.14 μg of BRU-2756 required

The data in Table 17 below show representative reagent quantities and radiochemical purity (RCP) values for reactions performed using these labeling conditions.

TABLE 17
Reactions Using a Ratio of 1 ¹⁷⁷Lu: 4 Ligand in the Presence
of 1 mg/mL Se-Met

| | Free | | | Reaction | | Radio- |
|---|---|---|---|---|---|---|
| BRU | Lu | RCP | Ligand | Volume | Activity | concentration |
| Number | (%) | (%) | (μg) | (μL) | (μCi) | (mCi/mL) |
| 2440 | 1.2 | 96.2 | 1.8 | 17.4 | 900 | 51.7 |
| 2440 | 3.2 | 93.7 | 2.0 | 20.1 | 1039 | 50.5 |
| 2443 | 2.2 | 92.2 | 2.1 | 19.7 | 1016 | 48.7 |
| 2443 | 4.3 | 88.3 | 2.5 | 23.6 | 1122 | 47.5 |
| 2624 | 2.3 | 94.9 | 2.1 | 20.3 | 1026 | 50.5 |
| 2624 | 3.8 | 93.5 | 2.1 | 20.0 | 1035 | 51.8 |
| 2625 | 4.5 | 93.2 | 2.1 | 20.0 | 1035 | 50.6 |
| 2625 | 6.1 | 92.6 | 2.1 | 20.0 | 1012 | 50.6 |

After dilution to a volume of 400 μL with a stabilizing mixture of 9 Saline: 1 Ascor L500: 0.1% HSA, RCP values greater than 90% at both time 0 and after the solutions were stored at 4° C. overnight were obtained for the majority of radiolabeled compounds prepared, and only a single product was formed. However, the reaction of ¹⁷⁷LuCl₃ with BRU-2443, BRU-2624 and BRU-2625 led to the formation of two slowly interconverting isomers in ratio of about 1:4. All the peptides that yielded two product peaks have a sarcosine directly linked to the DO3A10CM.

Metabolism Studies with ¹⁷⁷Lu-BRU-2813

In vitro and in vivo stability studies were performed with ¹⁷⁷Lu-BRU-2813 to determine its in vivo and in vitro metabolic stability. The following procedures were used.

### HPLC Analysis

The HPLC column, solvents, settings, flow rate, column temperature, and gradient used for the analysis of ¹⁷⁷Lu-BRU-2813 and its metabolites are as follows. Column: Zorbax Bonus-RP (4.6×250 mm; 5 μm, Agilent), Ratemeter: 1481LA with a 5×10³ scale, Solvents: A: H₂O/0.1% TFA (v:v); B: ACN/0.1% TFA (v:v), Flow rate: 1.0 mL/min, Column temperature: 30° C., Gradient:0% B/100% A for 0-5 min; ramped to 15% B/85% A at 6 min; and to 40% B/60% A at 66 min; back to 0% B/100% A at 67 min with a 10 min post run.

### Preparation of ¹⁷⁷Lu-BRU-2813 for In Vitro Stability Studies

¹⁷⁷Lu-BRU-2813 was prepared with a ratio of ligand to lutetium of 4 to 1. The amount of the required ligand was calculated based on the specific activity and quantity of ¹⁷⁷LuCl₃ that was used, as disclosed earlier. The ligand was dissolved at a concentration of 0.5 μg/μL “as is” in 0.2 M NaOAc (pH 4.8) buffer containing 10% DMSO and L-selenomethionine (1 mg/mL) as a radiostabilizer. The required amount of BRU-2813 was mixed with ˜5 μL (˜5 mCi) of ¹⁷⁷LuCl₃ (¹⁷⁷Lutetium (III) chloride (¹⁷⁷LuCl₃) dissolved in 0.05 N HCl at a concentration of ˜1 Ci/mL (Missouri University Research Reactor, MURR, Columbia, Mo.). Sufficient 0.2 M NaOAc buffer was added to bring the total volume to 0.12 mL. The mixture was heated at 100° C. for 10 min. After cooling the mixture to ambient temperature, normal saline solution was added into the reaction vial, to yield a final radioactivity concentration of 25 mCi/mL. The resulting ¹⁷⁷Lu-BRU-2813 formulation solution was immediately used for in vitro metabolism studies.

### Preparation of ¹⁷⁷Lu-BRU-2813 for In Vivo Stability Studies

¹⁷⁷Lu-BRU-2813 was prepared with a ligand to lutetium ratio of 4 to 1 as described above, but after cooling to ambient temperature, radiolysis protecting buffer (a 9:1 mixture of Bacteriostatic 0.9% Sodium Chloride Injection U.S.P. and ASCOR L500® Ascorbic Acid Injection U.S.P. containing 0.2% human serum albumin (final ascorbic acid concentration, 40 mg/mL) was added into the reaction vial, to yield a final radioactivity concentration of 1.0 mCi/mL. The RCP of ¹⁷⁷Lu-BRU-2813 (n=2) was found to be 93.3% and 92.4%, specific activity 1.05-1.06 Ci/μmol.

### In Vivo Metabolic Stability Studies for ¹⁷⁷Lu-BRU-2813 in Normal Mouse

At 10, 30 and 60 min post injection of ¹⁷⁷Lu-BRU-2813 (0.1 mCi, 0.1 mL) into normal mice, urine samples were collected and analyzed by HPLC. In addition, at both 2 and 10 min post injection, blood samples from two mice were collected. The collected blood samples at each time point were pooled and treated with two times their volume with ice-cooled methanol, and then the mixture was centrifuged at 4° C. for 20 min at 20,000×g to precipitate proteins. The supernatant was collected and the organic solvent in the solution was removed by speed-vacuum for 60 min. The concentrated supernatant was assayed by HPLC.

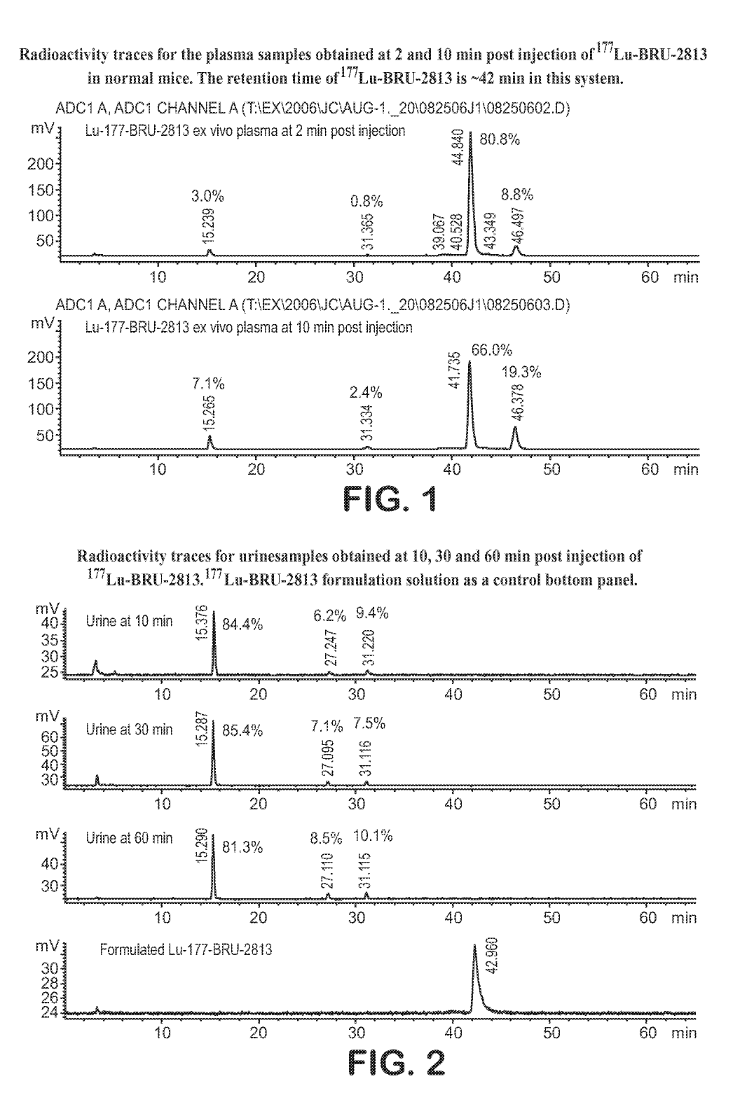

FIG. 1 shows the radioactivity traces for the plasma samples collected at 2 and 10 min post injection of ¹⁷⁷Lu-BRU-2813 in normal mice. Radiochromatograms for the urine samples collected at 10, 30 and 60 min post injection as well as the ¹⁷⁷Lu-BRU-2813 formulation solution as a control are shown in FIG. 2.

The HPLC results showed that 66% of the radioactivity remaining in the plasma at 10 min post injection of ¹⁷⁷Lu-BRU-2813 was still in parent form, while no ¹⁷⁷Lu-BRU-2813 was observed in the urine samples for any of the tested time points. These results suggested that despite the stabilization due to Darg in position 6 and the aza-Gly-NH₂ residue at the C-terminus, further attempts to stabilize the peptide in Lu-BRU-2318 (Lu-DOTA-Dnal2-Arg-Trp-Ser-His-Darg-Trp-Bpa4-Pro-azaGly-NH₂) against in vivo metabolism might be helpful.

### Preparation of Mouse Liver and Kidney Homogenates

Livers from 10 female Tac:NCr:Foxn1^{nu/nu} mice were excised, rinsed with ice cold PBS (no Ca⁺⁺ or Mg⁺⁺) and weighed. The tissues were minced with scissors in Petri dishes on ice with Tris buffered saline (Pierce BupH™ Tris buffered saline packs, #28376; 25 mM Tris, 150 mM NaCl, pH 7.2). After mincing, the tissues were dounce homogenized, and the total volume adjusted with ice cold Tris buffer (4 mL for every gram of excised tissue). The homogenate was centrifuged at ˜4000×g for 10 min at 4° C. The supernatants were aliquoted into Eppendorf tubes on ice (200 and 100 μl volumes) and stored at −80° C. Kidney homogenates were similarly prepared.

### In Vitro Metabolic Stability Studies for ¹⁷⁷Lu-BRU-2813 in Mouse Liver/Kidney Homogenate at 37° C.

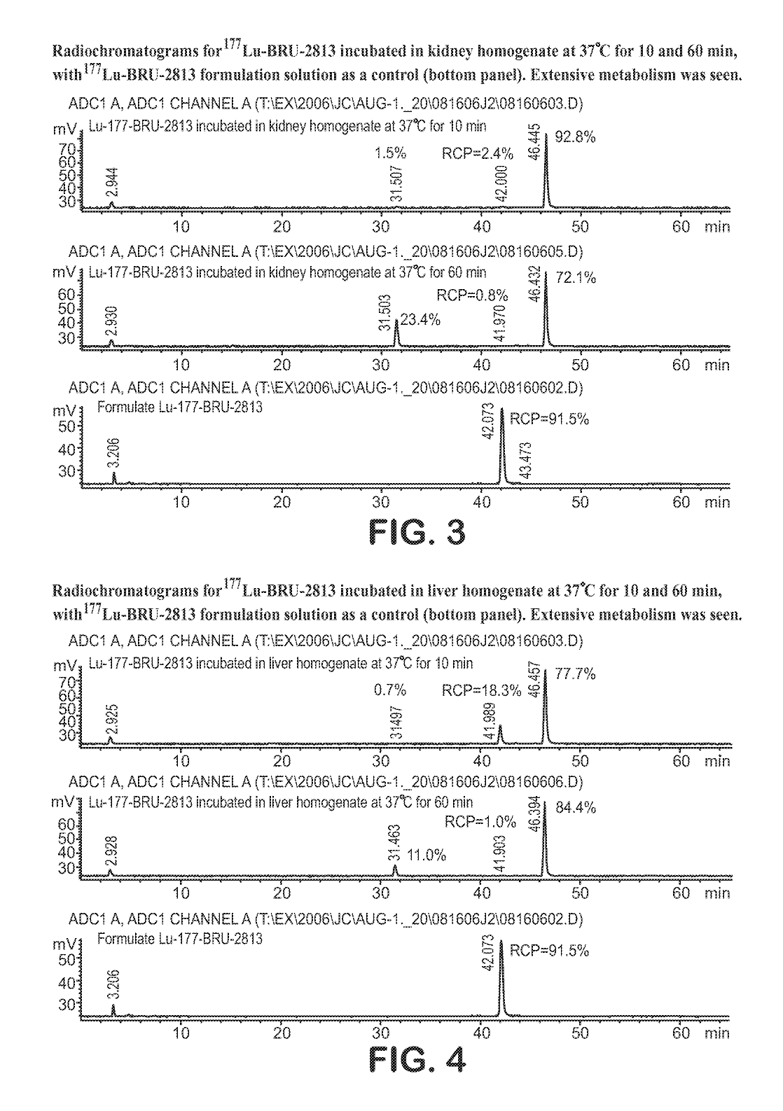

¹⁷⁷Lu-BRU-2813 (10 μL, 25 mCi/mL) prepared as described above was mixed with 100 μL of kidney or liver homogenate, and the mixture was incubated in a water-bath incubator at 37° C. After 10, 30 and 60 min, 20 μL of the mixture was removed and mixed with 40 μL of ice-cold methanol. The mixture was centrifuged at 4° C. for 20 min at 20,000×g to precipitate the protein; 10 μL (˜7.6 μCi) of the supernatant was injected into the HPLC for the analysis. The results obtained are shown in FIG. 3 and FIG. 4, which show the radiochromatograms of ¹⁷⁷Lu-BRU-2813 incubated in kidney and liver homogenates, respectively, at 37° C. for 10 and 60 min. Two major metabolites with retention times of ˜31 min and ˜46 min (retention time for ¹⁷⁷Lu-BRU-2813 was ˜42 min) were observed for ¹⁷⁷Lu-BRU-2813 incubated in either kidney or liver homogenate. Peaks at these retention times were also observed in the in vivo plasma sample (FIG. 1).

These data, like those obtained following in vivo administration, again suggested that improvements in metabolic stability might be helpful.

### Identification of the Metabolites of ¹⁷⁵Lu-BRU-2813 by LC/MS

Non-radioactive Lu-BRU-2318 was prepared using a ratio of ¹⁷⁵Lu to BRU-2813 of 1.2 to 1. Briefly, BRU-2813 was dissolved in 0.2 M (pH 4.8) NaOAc buffer containing 10% DMSO (v:v) at a concentration of 1 μg/μL “as is”. An 0.2 mL aliquot of BRU-2813 solution (1 mg/mL) was mixed with 2.2 μL (2.2 μg Lu, 1.2 equivalents) of a lutetium plasma standard solution (Alfa Aesar, Lu₂O₃, 10 mg/mL in 5% HNO₃). The mixture was heated at 100° C. for 10 min, and then cooled to ambient temperature in a water bath for 2 min. The sample was analyzed by HPLC to confirm that all BRU-2813 ligand was coordinated by Lu.

An aliquot (40 μL, 2.5 mg/mL) of non-radioactive Lu-BRU-2813 prepared as described above was mixed with kidney homogenate stock solution (200 μL); the mixture was incubated in a water-bath incubator at 37° C. The final concentration for the Lu-BRU-2813 in the homogenate sample solution was ˜0.4 mg/mL. After 60 min incubation, the sample was immediately cooled on ice, and then 0.48 mL of ice-cooled methanol was added. The sample was centrifuged at 4° C. for 20 min at 20,000×g to precipitate the protein. The supernatant was collected, concentrated using a speed vacuum to remove organic solvents, and then analyzed by LC/MS.

Mass spectra were recorded on an Agilent 1100 LC/MSD instrument in the atmospheric pressure ionization electrospray (API-ES) positive mode. The HPLC settings used for the LC/MS analysis are as follows. Column. Phenomenex (2.0×250 mm; 4 μm), Solvents: A: H₂O/0.1% TFA/0.1% Formic acid (v:v:v); B: ACN, Flow rate: 0.4 mL/min, Column temperature: 37° C., Gradient: Ramp from 12% B to 32% B over 20 min; ramp from 32% B to 100% B at 25 min; hold at 100% B from 25-29 min; return to 12% B/88% A by 30 min with a 10 min post run.

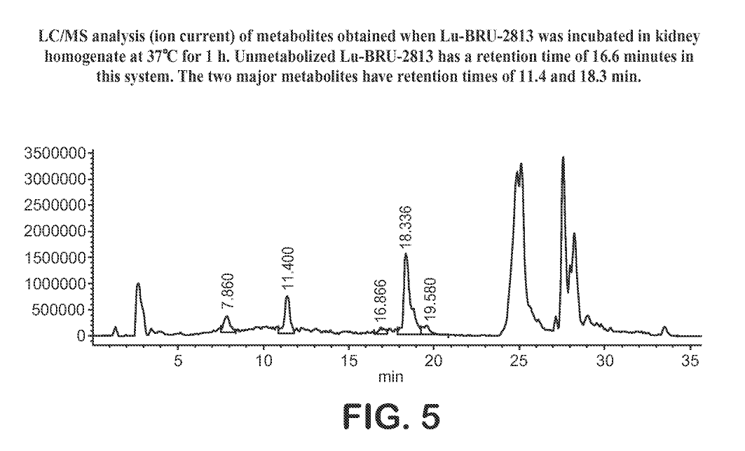

FIG. 5 and the table below show the LC/MS analytical results for ¹⁷⁵Lu-BRU-2813 following incubation for 1 h at 37° C. in mouse kidney homogenate. As had been observed with samples of ¹⁷⁵Lu-BRU-2813, two major metabolites formed, one that was more polar than starting material (11.4 min) and one that was retained on the column longer than Lu-BRU-2813 (retention time 18.4 min) The mass spectral data showed a molecular weight of 1933.7 for the peak at 18.4 min [(M+3H⁺/3)=645.4; (M+2H⁺/2=967.8; and (M+H⁺)=1934.7], and a molecular weight of 1681.5 for the peak at 11.4 min [(M+3H⁺)/3=561.7; (M+H⁺)/2=841.7; (M+H⁺)=1682.5].

As summarized in Table 18 below, the ions observed by LC/MS following metabolism of ¹⁷⁵Lu-BRU-2813 in kidney homogenate appear to correspond to cleavage between the biphenylalanyl (Bpa4) and proline (Pro) residues (Metabolite 1) and cleavage between Trp and Bpa4 residues (Metabolite 2).

TABLE 18
Summary of LC/MS Analytical Results for Lu-BRU-2813 Incubated
in Kidney Homogenate at 37° C. for 1 h

| | Lu-BRU-2813 | Metabolite-1 | Metabolite-2 |
|---|---|---|---|
| Formula | C₉₄H₁₁₅N₂₆O₁₉Lu | C₈₈H₁₀₅N₂₂O₁₈Lu | C₇₂H₉₂N₂₁O₁₆Lu |
| Sequence | Lu-DOTA- | Lu-DOTA- | Lu-DOTA- |
| | Dnal2RWSHrWBpa4P- | Dnal2RWSHrWBpa4- | Dnal2RWSHrW-OH |
| | azaG-NH₂ | OH | |
| RT on LC/MS | ~16.8 min | ~18.3 min | ~11.4 min |
| Expected mw | 2088.1 | 1933.9 | 1682.6 |
| Measured (M + H⁺) | 2089.6 | 1934.7 | 1682.5 |

These results suggested that metabolic stabilization around the Bpa4-Pro residues in Lu-BRU-2813 might serve to stabilize this LHRH derivative.

### LHRH-II Analogs Designed to Increase Metabolic Stability

The results of metabolism studies with ¹⁷⁷Lu-BRU-2813 described above showed that this construct undergoes metabolism in vivo in mice. It was observed that only 66% of the radioactivity remaining in the plasma at 10 min post injection was still ¹⁷⁷Lu-BRU-2813, and no intact ¹⁷⁷Lu-BRU-2813 was observed in the urine samples collected 10, 30 and 60 min post injection. In vitro studies in kidney and liver homogenates showed that after 10 min incubation at 37° C., only 2.4% of the radioactivity in kidney homogenate and 18.3% in liver homogenate was the parent compound ¹⁷⁷Lu-BRU-2813.

In both in vivo and in vitro studies, the main metabolite resulted from the cleavage of the bond between the amino acids at positions 8 and 9. Accordingly, derivatives of the LHRH-II analogs BRU-2477 and BRU-2813 were synthesized and tested for metabolic stability. All of the derivatives had modifications at position 9 to inhibit cleavage between positions 8 and 9.

### Solid-Phase Synthesis of Peptides

LHRH II peptides prepared to improve metabolic stability were synthesized following the general procedure developed as previously described herein for the synthesis of peptides on the solid phase. All the peptides tested, except BRU-2447, BRU-3122, BRU-3123 and BRU-3124, contain a DO3A10CM chelator at the N-terminal BRU-3122, BRU-3123 and BRU-3124 are analogs of BRU-2477 that were synthesized based on the results obtained with the derivatives of BRU-2813. BRU-3046 and BRU-3064 are metabolites of BRU-2813 that had been observed in previous in vivo metabolism studies. Peptides BRU-3081 and BRU-3122, containing a Ψ(CH₂N)Pro modification in the sequence, were prepared by incorporating the corresponding -AA-Ψ(CH₂N)Pro- during synthesis.

To accomplish the syntheses of these peptides, the crucial intermediates Fmoc-Bpa-Ψ(CH₂N)Pro-OH (4) and Fmoc-Tyr(Bz)-Ψ(CH₂N)-Pro-OH (7) were synthesized from the corresponding Fmoc-Bpa-OH and Fmoc-Tyr(Bz)-OH as shown in Schemes 6 and 7. Synthesis of Fmoc-Bpa4-Ψ(CH₂N)Pro-OH (4)

The dipeptide Fmoc-Bpa4-Pro-OtBu (1) prepared from Fmoc-Bpa-OH and H-Pro-OtBu was subjected to BH₃-THF reduction which yielded amide-carbonyl reduced product 2 with concomitant reduction of the benzoyl group of the benzophenone function to a hydroxyl group (Scheme 1). To convert the alcohol function in 2 back to a benzoyl group, product 2 was treated/oxidized with MnO₂ in CH₂Cl₂ providing the psi-peptide, Fmoc-Bpa4-Ψ(CH₂N)Pro-OtBu (3). Finally the t-butyl group in 3 was removed by treating with trifluoroacetic acid:phenol:water cleavage cocktail to provide the required psi-dipeptide, Fmoc-Bpa4-Ψ(CH₂N)Pro-OH (4).

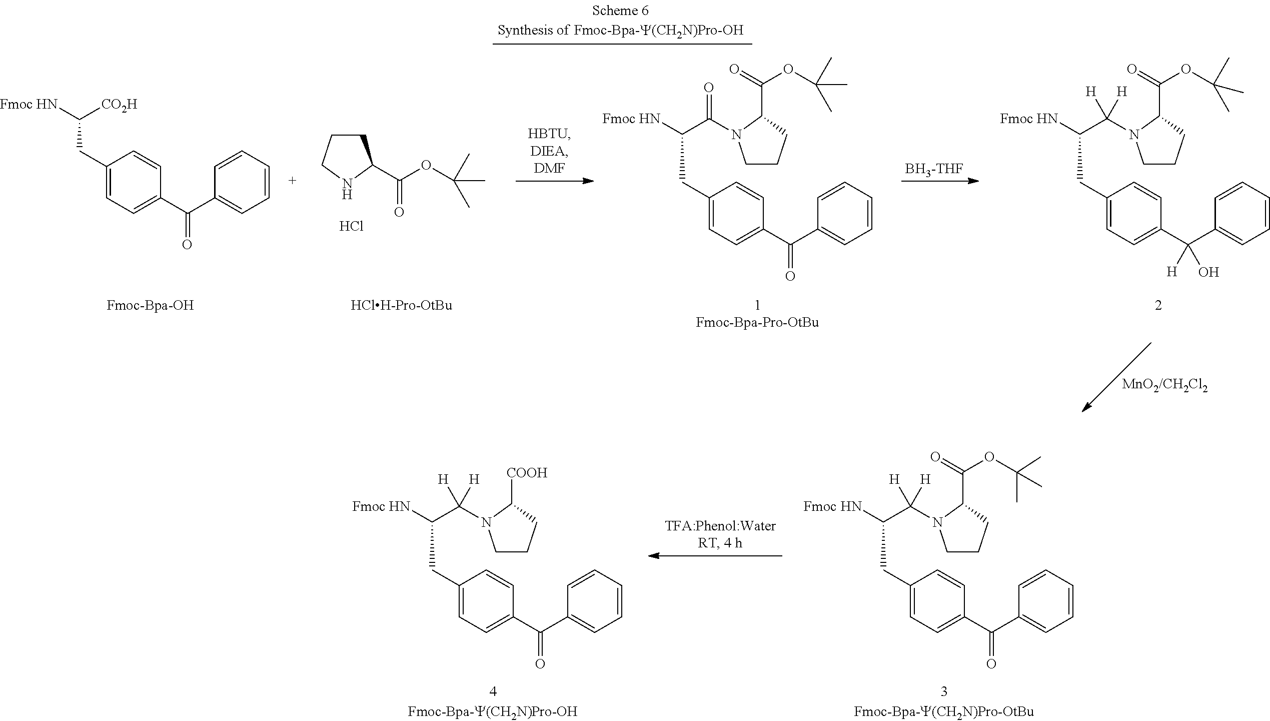

### Synthesis of Fmoc-Bpa4-Ψ(CH₂N)Pro-OH (4) (Scheme 6)

Fmoc-Bpa4-Pro-OtBu (1). Diisopropylethylamine (0.57 g, 0.8 mL, 4.4 mmol) was added to a mixture of Fmoc-Bpa-OH (1.0 g, 2.0 mmol), HATU (0.8 g, 2.1 mmol) and L-proline t-butyl ester hydrochloride (0.45 g, 2.16 mmol) in DMF (4.0 mL) and the mixture was stirred for 12 h. DMF was removed and the residue was treated with a solution of sodium carbonate (5%) and extracted with ethyl acetate. The ethyl acetate solution was washed with water and dried (Na₂SO₄). Evaporation of ethyl acetate gave an oil which was dried under vacuum to give a foamy solid. The crude dipeptide was purified by silica gel column chromatography using hexane/ethyl acetate (7/3). Fractions (R_{f}=0.4) were collected and evaporated to provide the dipeptide Fmoc-Bpa4-Pro-OtBu (1) as a foamy solid. Yield: 0.9 g (69%). MS (M+H)⁺=645.4

Peptide (2). To a solution of the dipeptide 1 (100 mg, 0.15 mmol) in THF (1.0 mL) was added BH₃-THF complex (1 M solution, 1.0 mL) and the mixture was stirred for 30 min. Excess BH₃-THF complex was decomposed by adding methanol. Citric acid (50 mg) was added to the solution and the solvents were removed to give an oil. The oil was dissolved in ethyl acetate and washed with water and dried (Na₂SO₄). Evaporation of ethyl acetate provided an oil, which was purified by silica gel column chromatography using CH₂Cl₂/CH₃OH (95/5). Product-containing fractions were collected and evaporated to provide an oil, which was dried under vacuum to provide 2 as a foamy solid. Yield: 0.052 g (53%). MS (M+H)⁺=633.4

Fmoc-Bpa4-Ψ(CH₂N)Pro-OtBu (3). MnO₂ (1.2 g) was added to a solution of the compound 2 (450 mg, 0.76 mmol) in CH₂Cl₂ (15 mL) and the mixture was stirred for 24 h.

Additional MnO₂ (500 mg) was then added and the stirring was continued for additional 24 h. MnO₂ was filtered and the CH₂Cl₂ solution was concentrated, and the crude product was purified by silica gel column chromatography using CH₂Cl₂/ethyl acetate (8/2). UV visible fractions (R_{f}=0.2) were collected and evaporated to give the ketone Fmoc-Bpa4-Ψ(CH₂N)Pro-OtBu (3) as a thick oil, which was evaporated to give a foamy solid. Yield: 0.350 g (73%). MS (M+H)⁺=631.2

Fmoc-Bpa4-Ψ(CH₂N)Pro-OH (4). TFA (5.0 mL), phenol (100 mg) and water (0.2 mL) was added to Fmoc-Bpa4-Ψ(CH₂N)Pro-OtBu (3) (0.4 g, 0.632 mmol) and the mixture was stirred for 5 h. TFA was removed and the residue was diluted with water and purified by preparative HPLC using CH₃CN/Water containing 0.1% TFA. Pure fractions were collected and freeze dried to give the dipeptide Fmoc-Bpa4-Ψ(CH₂N)Pro-OH (4) as a fluffy solid. Yield 230 mg (63%). MS (M+H)⁺=577.2

### Synthesis of Fmoc-Tyr(Bz)-ω(CH₂N)Pro-OH (7)

Following the procedure described for the synthesis of Fmoc-Bpa-T(CH₂N)Pro-OH (4), the required intermediate Fmoc-Tyr(Bz)-Ψ(CH₂N)Pro-OH (7) was prepared from Fmoc-Tyr(Bz)-OH and H-Pro-OtBu as shown in Scheme 7.

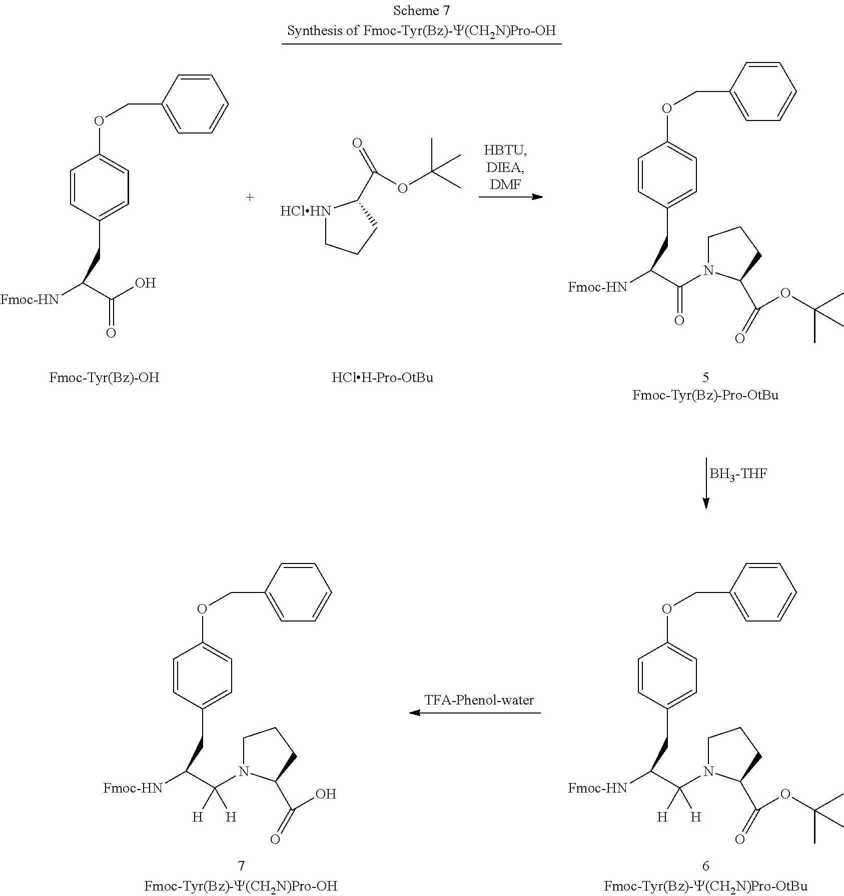

### Synthesis of Fmoc-Tyr(Bz)-Ψ(CH₂N)Pro-OH (7) (Scheme 7)

Fmoc-Tyr(Bz)-Pro-OtBu (5). Diisopropylethylamine (2.34 g, 3.25 mL, 18.0 mmol) was added to a mixture of Fmoc-Tyr(Bz)-OH (2.0 g, 4.05 mmol) and proline t-butyl ester hydrochloride (1.24 g, 6.0 mmol) and HATU (2.3 g, 6.0 mmol) and the mixture was stirred for 4 h. The reaction mixture was then poured into water and the pasty solid obtained was dissolved in ethyl acetate and extracted with ethyl acetate, washed with water and dried (Na₂SO₄). Evaporation of the combined ethyl acetate solution gave a foamy solid, which was purified by silica gel column chromatography using CH₂Cl₂/CH₃OH (95/5). Fractions (R_{f}=0.6) were collected and evaporated to give dipeptide Fmoc-Tyr(Bz)-Pro-OtBu (5) as a foamy solid. Yield: 2.2 g (85%). MS (M+H)⁺=647.2

Fmoc-Tyr(Bz)-'P(CH₂N)Pro-OtBu (6). BH₃-THF complex (1.0 M solution, 10.0 mL, 10 mmol) was added to a solution of the dipeptide Fmoc-Tyr(Bz)-Pro-OtBu (5) (1.29 g, 1.99 mmol) in dry THF (5.0 mL) and the mixture was stirred for 10 h. The reaction mixture was quenched by the addition of methanol and the solvents were removed. The residue was treated with ammonium chloride solution (5%, 100 mL) and extracted with ethyl acetate. The ethyl acetate solution was washed with water and dried. Evaporation of ethyl acetate gave an oil which was purified by silica gel column chromatography using CH₂Cl₂/CH₃OH (95/5). Fractions (R_{f}=0.6) were collected and evaporated to provide Fmoc-Tyr(Bz)-Ψ(CH₂N)Pro-OtBu (6) as a foamy solid. Yield: 0.58 g (48%). MS (M+H)⁺=633.2

Fmoc-Tyr(Bz)-Ψ(CH₂N)Pro-OH (7). TFA (5.0 mL), phenol (100 mg) and water (0.2 mL) was added to Fmoc-Tyr(Bz)-Ψ(CH₂N)Pro-OtBu (6) (0.4 g, 0.632 mmol) and the mixture was stirred for 5 h. TFA was removed and the residue was diluted with water and purified by preparative HPLC using CH₃CN/H₂O containing 0.1% TFA. Pure fractions were collected and freeze dried to give Fmoc-Tyr(Bz)-Ψ(CH₂N)Pro-OH (7) as a fluffy solid. Yield: 230 mg (63%). MS (M+H)⁺=577.2

### Preparation of ¹⁷⁵Lu-LHRH II and Unlabeled LHRH Analogs for In Vitro Metabolic Stability Studies

The proline-modified DO3A10CM-derivatized LHRH peptide analogs were dissolved in 50% ACN/50% H₂O (v: v) at a concentration of 2 μg/μL. An 0.15 mL (300 μg) aliquot of the peptide solution was mixed with 20 μL of 1 M NaOAc pH 5.1 buffer and sufficient lutetium standard solution (Lu₂O₃, 10 mg/mL in 5% HNO₃) to achieve a ratio between peptide and Lu of 1:1. The solution was heated at 100° C. for 15 min, and then cooled to ambient temperature in a water bath for 2 min. The yield of the reaction was determined by HPLC.

For the ligands BRU-2447, BRU-3122, BRU-3123 and BRU-3124 that do not contain a DO3A10CM chelator, the samples were prepared as described above but substituting the lutetium standard solution with the same volume of 5% HNO₃.

### Preparation of ¹⁷⁷Lu-LHRH II Analogs for In Vitro Metabolic Stability Studies

Labeling of the LHRH II analogs with ¹⁷⁷Lu was achieved using a ratio between ligand and lutetium of 4 to 1 (the amount of ligand used was calculated based on the specific activity of ¹⁷⁷LuCl₃). The required amount of ligand (2 μg/μL “as is”) dissolved in 50% ACN/50% H₂O (v: v) was mixed with ˜5 μl (˜5 mCi) of ¹⁷⁷LuCl₃ and the volume of 0.2 M NaOAc buffer pH 4.8 to reach a final volume of 0.11 mL. The mixture was heated at 100° C. for 10 min and, after cooling to room temperature, the ¹⁷⁷Lu-LHRH II analog solution was immediately used in the in vitro metabolism studies.

### In Vitro Metabolism of Lu-LHRH II Analogs in Mouse Liver Homogenate

An 18 μL aliquot of the ¹⁷⁵Lu-LHRH-II analog solution (prepared as described earlier herein) with or without the addition of 2 μL of ¹⁷⁷Lu-LHRH II analog solution (prepared as described earlier herein) was mixed with 100 μL of liver homogenate and incubated at 37° C. in a water-bath incubator. After 0 and 60 min, the sample was removed from the incubator, immediately cooled on ice and 2 μL of 10 mM EDTA and 0.2 mL of ice-cooled MeOH were added, mixing after each addition. The proteins in the sample were separated by centrifugation at 14,000 rpm for 20 min. The supernatant was harvested and, in the samples spiked with the ¹⁷⁷Lu-analog, the radioactivity was measured using a Capintec dose calibrator to determine recovery. The sample was then analyzed by HPLC.

The addition of the ¹⁷⁷Lu-labeled analog helped in the identification of the cold metabolites because the radioactivity trace, unlike the UV trace, did not show all the peaks generated during incubation in the liver homogenate. The UV peaks coeluting with the radioactive ones were identified as Lu-containing metabolites.

### Identification of Metabolites of Lu-LHRH II Analogs by LC/MS

An 18 μL aliquot of the ¹⁷⁵Lu-LHRH II analog solution prepared as described earlier herein was mixed with 100 μL of liver homogenate and incubated at 37° C. in a water-bath incubator. The final concentration of the ¹⁷⁵Lu-LHRH II analog in the homogenate sample solution was 0.3 mg/mL. After 0 and 60 min, the sample was removed from the incubator, immediately cooled on ice and 2 μL of 10 mM EDTA and 0.24 mL of ice-cooled methanol was added, mixing after each addition. The proteins in the sample were separated by centrifugation at 14,000 rpm for 20 min. The supernatant was collected and analyzed by LC/MS as described earlier herein.

### LHRH-II Analogs Used in Stability Analysis

The Lu complexes tested were derivatives of BRU-2813 containing the sequence DO3A10CM-Dnal2-Arg-Trp-Ser-His-Darg-Trp-Bpa4-aa⁹-azaGly-NH₂. Based on the results obtained in these tests, derivatives of BRU-2477 with the same substitutions at position 9 were also synthesized and their metabolic stability tested. The BRU-2477 derivatives were of the general sequence pGlu-His-Trp-Ser-His-Darg-Trp-Tyr-aa⁹-azaGly-NH₂.

Table 19 lists relevant information for the LHRH-II analogs used in the stabilization studies. Names, abbreviations and structures of unnatural amino acids used in the syntheses of these peptides are shown in Table 20.

TABLE 19
Summary of the LHRH II Analogs Used in the Stabilization Studies
Summary of the LHRH II analogs

| Analogs of | | | |
|---|---|---|---|
| BRU-2477 | pGlu-His-Trp-Ser-His-Darg-Trp-aa8-aa9-azaGly-NH₂ | | |
| Ligand | MW | EC₅₀ [μM] | -aa8-aa9- |
| BRU-2477 | 1335 | 0.84 | -Tyr-Pro- |
| BRU-3122 | 1322 | 3.13 | Tyr-Ψ(CH₂N)-Pro- |
| BRU-3123 | 1351 | 0.99 | Tyr-Ampc4- |
| BRU-3124 | 1352 | 2.49 | -Tyr-Thz- |
| Analogs of | DO3A10CM-Dnal2-Arg-Trp-Ser-His- | | |
| BRU-2813 | Darg-Trp-aa8-aa9-azaGly-NH₂ | | |
| Ligand | MW | EC₅₀ [μM] | -aa8-aa9- |
| BRU-2813 | 1916 | 0.42 | -Bpa4-Pro- |
| BRU-2993 | 1902 | 0.38 | -Bpa4-Aze- |
| BRU-2994 | 1934 | 0.60 | -Bpa4-Flp4- |
| BRU-2995 | 1931 | 0.41 | -Bpa4-Ampt4- |
| BRU-2996 | 1930 | 0.35 | -Bpa4-Pip- |
| BRU-3072 | 1876 | 0.16 | Bpa4-Thz- |
| BRU-3081 | 1902 | 0.30 | -Bpa4-Ψ(CH₂N)Pro |
| BRU-3046 | 1511 | Metabolite standard | BRU-2813_{[1-7]}-OH |
| | | 15.85 | |
| BRU-3064 | 1762 | Metabolite standard | BRU-2813_{[1-8]}-OH |
| | | 0.72 | |

TABLE 20
Names, Abbreviations and Structures of Unnatural Amino Acids

| Name | Abbreviation | Structure |
|---|---|---|
| 4-Benzoyl- L-phenylalanine | Bpa4 | 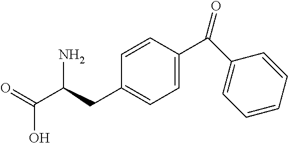 |
| L-Pyroglutamic acid | pGlu | 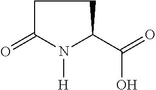 |
| Azaglycine amide | AzaGly-NH₂ | 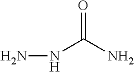 |
| 2-Naphthyl- D-alanine | Dnal2 | 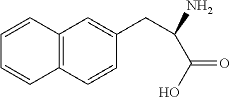 |
| Bpa4-Pro Psi-dipeptide | Bpa4-Ψ(CH₂N)-Pro | 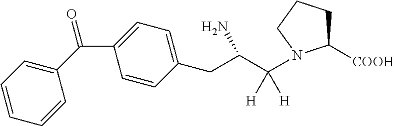 |
| Tyr-Pro Psi dipeptide | Tyr-Ψ(CH₂N)-Pro | 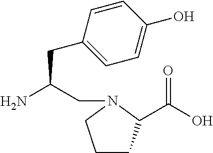 |
| 4-(S)-cis- Amino-L-proline | Ampc4 | 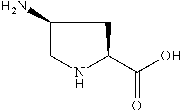 |
| 4-(R)-trans- Amino-L-proline | Ampt4 | 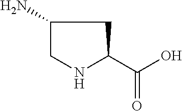 |
| L-4-Thiaproline | Thz | 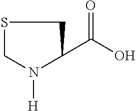 |
| 4-(S)-Fluoro- L-proline | Flp4 | 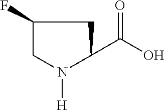 |
| L-2-Azetidine- carboxylic acid | Aze | 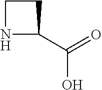 |
| L-Pipecolic acid | Pip | 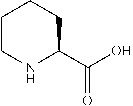 |

### In Vitro Metabolic Stability Studies for Lu-LHRH-II Analogs Incubated in Mouse Liver Homogenate

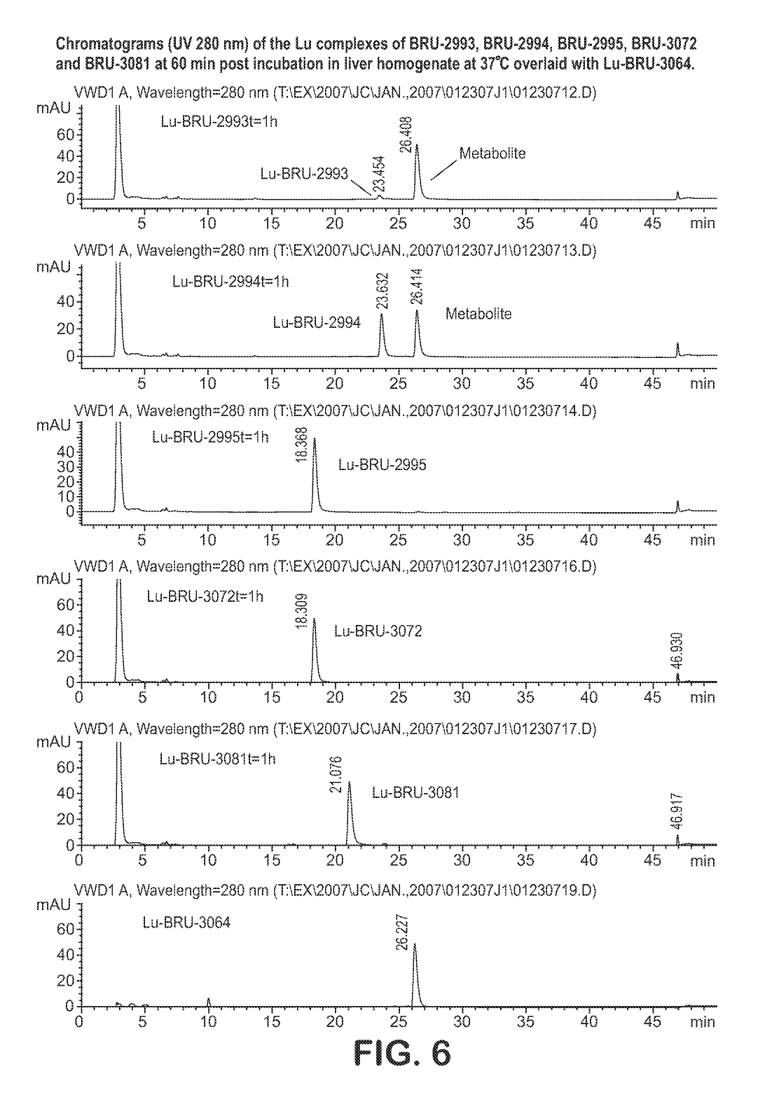

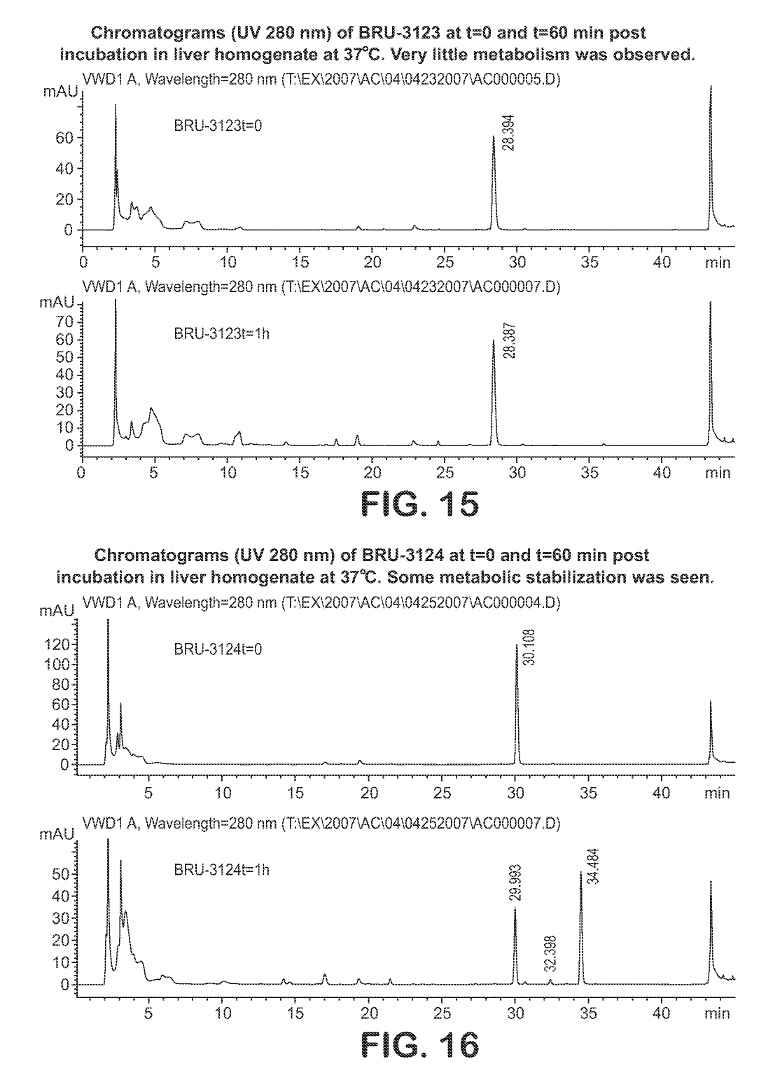

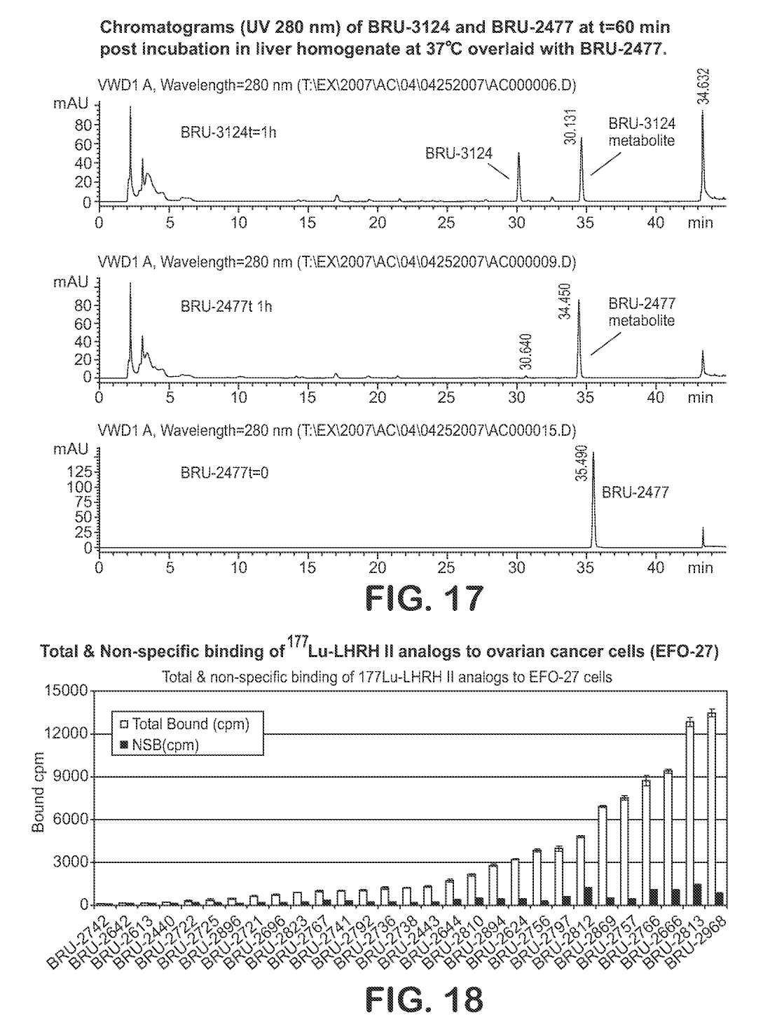

FIGS. 6-17 following depict the results of the stability studies on the various analogs shown in Table 19. These figures show chromatographic elution profiles, based on monitoring at A₂₈₀, of the peptides tested in this study, as well as the metabolites generated from the peptides.

FIG. 6 shows the UV traces at 280 nm of the Lu-complexes of BRU-2993, BRU-2994, BRU-2995 and BRU-3072 after incubation in liver homogenate at 37° C. for 60 min, overlaid with the UV trace of Lu-BRU-3064, the metabolite of Lu-BRU-2813 observed in previous metabolism studies. As stated previously, no metabolism was observed for the lutetium complexes of BRU-2995, BRU-3072 and BRU-3081. The metabolite observed with the Lu-1146375-0002 complexes of BRU-2993 and BRU-2994 was the expected lutetium complex of BRU-3064 (DO3A10CM-Dnal2-Arg-Trp-Ser-His-Darg-Trp-Bpa-OH) previously observed in the in vivo metabolism studies with ¹⁷⁷Lu-BRU-2813.

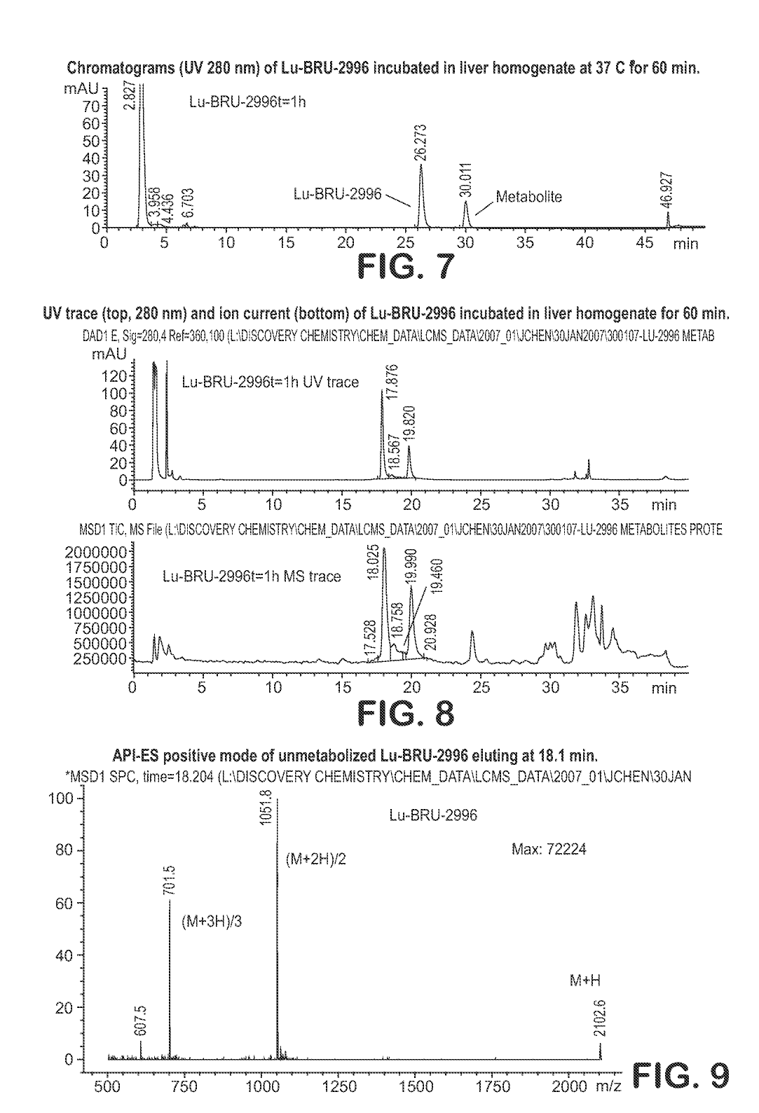

FIG. 7 shows the UV trace at 280 nm of the Lu-complex of BRU-2996 after incubation in liver homogenate at 37° C. for 60 min. The metabolite formed with Lu-BRU-2996 was not the expected Lu-BRU-3064, indicating that cleavage between aa8 and aa9 did not occur for this compound.

### Identification of the Metabolite of Lu-BRU-2996 by LC/MS

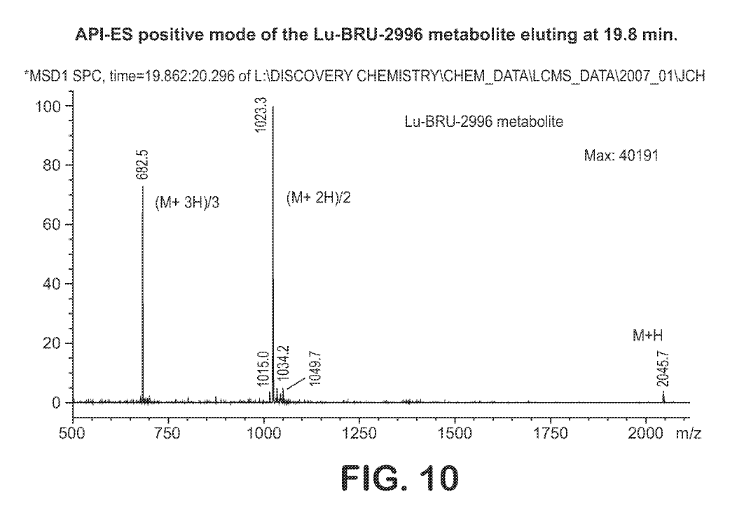

FIG. 8 shows the UV and the ion-current trace for Lu-BRU-2996 following incubation in liver homogenate at 37° C. for 60 min. The positive-ion spectrum of the peak eluting at 18.1 min (FIG. 9) displayed the protonated molecular ion [M+H]⁺ at m/z 2102.6 and was identified as Lu-BRU-2996. The positive-ion spectrum of the peak eluting at 19.8 min (FIG. 10) displayed the protonated molecular ion [M+H]⁺ at m/z 2045.7, which corresponds to the mw expected for Lu-DO3A10CM-Dnal2-Arg-Trp-Ser-His-Darg-Trp-Bpa4-Pip-OH.

### Identification of the Metabolite of BRU-2477 by LC/MS

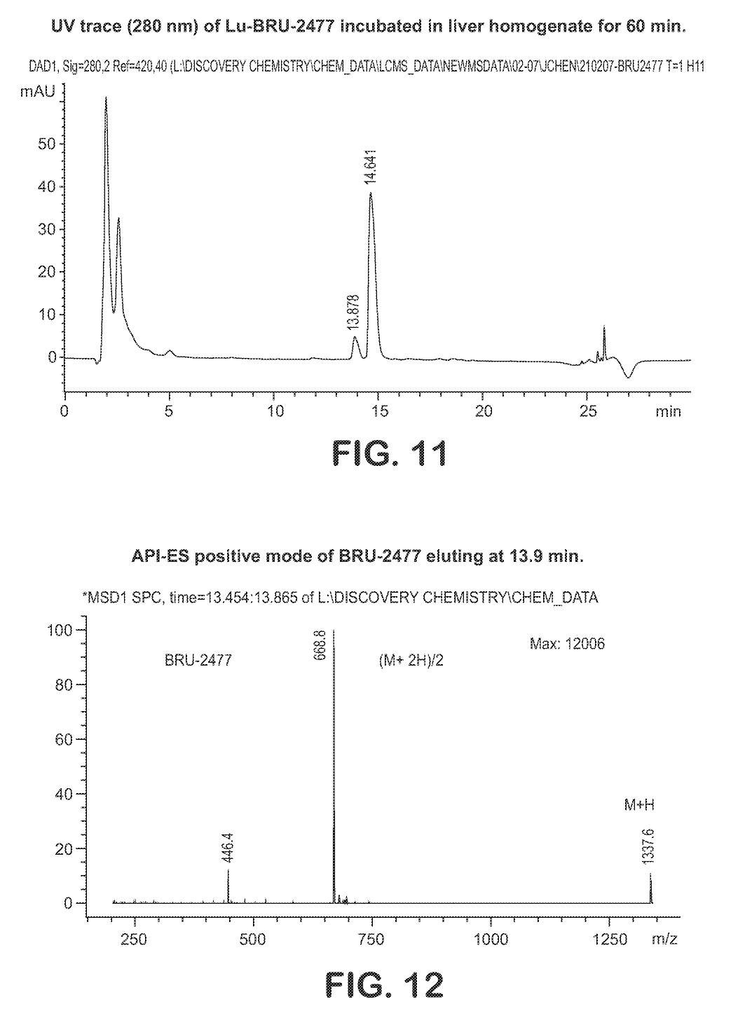

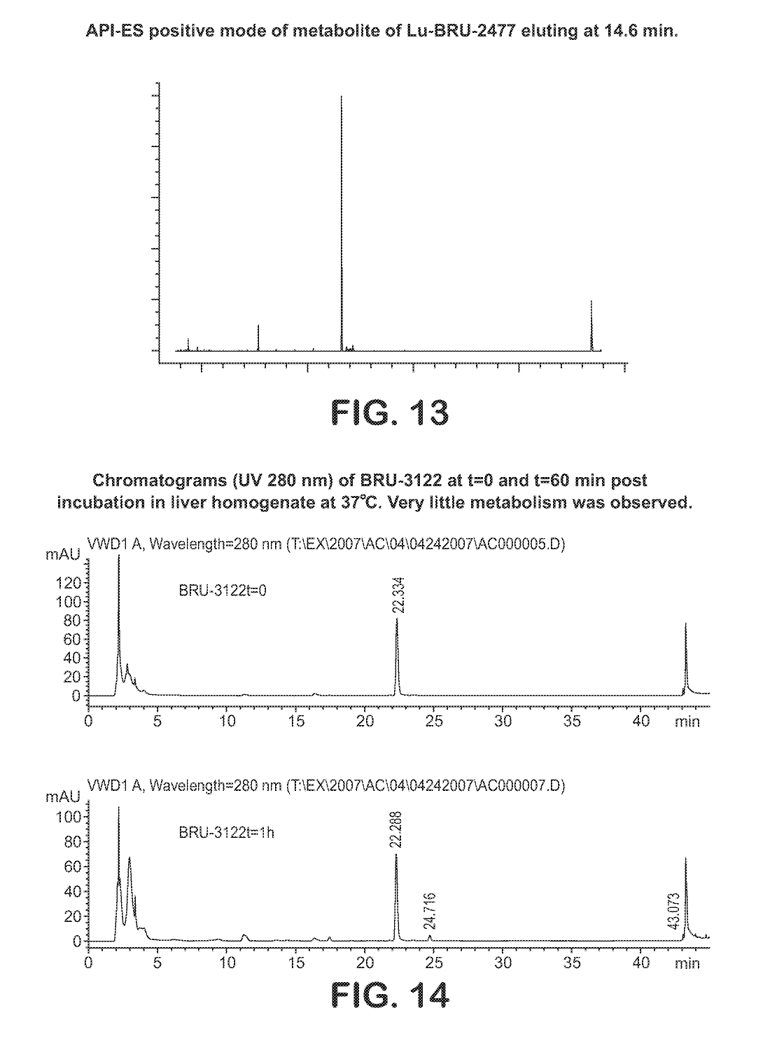

FIG. 11 shows the UV trace of BRU-2477 incubated in liver homogenate at 37° C. for 60 min. The positive-ion spectrum of the peak eluting at 13.9 min (FIG. 12) displayed the protonated molecular ion [M+H] at m/z 1336.6 and was identified as BRU-2477. The major product was a metabolite at a retention time of 14.6 min. The positive-ion spectrum of the peak eluting at 14.6 min (FIG. 13) displayed a protonated molecular ion [M+H] at m/z 1182.4, corresponding to the mw expected for pGlu-His-Trp-Ser-His-Darg-Trp-Tyr-OH (BRU-2996).

### In Vitro Metabolic Stability Studies of the Analogs of BRU-2477 Incubated in Mouse Liver Homogenate

Based on the results with the derivatives of BRU-2813 having modifications between aa8-aa9, derivatives of BRU-2477 were synthesized in the attempt to create a metabolically stable compound. Table 21 illustrates the modifications in the derivatives synthesized.

TABLE 21

| Ligand | aa8-aa9 |
|---|---|
| BRU-2477 | Tyr-Pro- |
| BRU-3122 | Tyr-Ψ(CH₂N)-Pro- |
| BRU-3123 | Tyr-Ampc4- |
| BRU-3124 | Tyr-Thz- |

The metabolic stability of these peptides was tested in triplicate following the procedures described earlier herein.

The chromatograms in FIG. 14 show that there was no metabolism of BRU-3122 in liver homogenate after 60 min at 37° C. Likewise, the chromatograms in Error! Reference source not found.15 demonstrate that no metabolism of BRU-3123 occurred in liver homogenate. However, the chromatogram at t=60 min in Error! Reference source not found.16 shows the formation of an additional peak, indicating metabolism of BRU-3124 in liver homogenate.

The experiments identifying the BRU-2477 metabolite by LC/MS showed that BRU-2477 was cleaved between Tyr⁸ and Pro⁹. If BRU-3124 was cleaved between the tyrosine in position 8 and the thiaproline in position 9, the final metabolite should be the same for BRU-2477 and BRU-3124.

To verify this hypothesis, BRU-2477 was incubated with liver homogenate as previously described for BRU-3124. The chromatograms seen in Error! Reference source not found.17 strongly suggest that the same metabolite was formed with BRU-2477 and BRU-3124.

As can be seen from these data, the substitution of the BRU-2813 Pro in position 9 with Ψ(CH₂N)-Pro, Thz or Ampt4 completely stopped metabolism without decreasing the binding affinity of the resulting derivative. The substitution of Pro at position 9 with Pip or Flp4 resulted in significant improvement in stability with minimal effect on binding affinity compared to the BRU-2813 parent.

The BRU-2477 derivatives containing Ψ(CH₂N)-Pro (BRU-3122) or Ampc4-(BRU-3123) in position 9 were found to be completely metabolically stable, while BRU-3124, with Thz in position 9, was seen to be partially stabilized relative to BRU-2477. This indicated that for the BRU-2813 derivative with a Thz in position 9 (BRU-3072), the presence of Bpa4 in position 8 contributed to the metabolic stabilization. Of the three derivatives of BRU-2477, only BRU-3123 maintained binding affinity similar to BRU-2477.

In Table 22 are summarized the results of the stability analyses of the Lu-labeled and unlabeled LHRH-II analogs incubated in liver homogenates at 37° C. for 60 min.

TABLE 22
Stability Comparison of the Lu-Labeled LHRH-II Analogs Incubated in Liver
Homogenateat 37° C. for 60 min (% of parent remaining) (mean ± SD) (n = 3)

| | | | | | Stabilized |
|---|---|---|---|---|---|
| | % | | | | relative to |
| BRU Number | remaining | -aa8-aa9- | P value* | EC50 | parent? |
| Lu-BRU-2813 | 12.4 ± 8.7 | -Bpa4-Pro- | | 0.33 | |
| Unstabilized | | | | | |
| parent | | | | | |

“Metabolically Stabilized” Compounds of Form
DO3A10CM-Dnal2-Arg-Trp-Ser-His-Darg-Trp-AA⁸-AA⁹-azaGly-NH₂

| | | | | | |
|---|---|---|---|---|---|
| Lu-BRU-2993 | 14.9 ± 13.2 | -Bpa4-Aze- | 0.8 | 0.38 | not |
| | | | | | significant |
| Lu-BRU-2994 | 45.7 ± 1.2 | -Bpa4-Flp4- | 0.003 | 0.60 | Yes |
| Lu-BRU-2995 | 98.7 ± 0.1 | -Bpa4-Ampt4 | <0.001 | 0.41 | Yes |
| Lu-BRU-2996 | 71.7 ± 2.9 | -Bpa4-Pip- | <0.001 | 0.35 | Yes |
| Lu-BRU-3072 | 98.5 ± 0.2 | -Bpa4-Thz- | 0.001 | 0.16 | Yes |
| Lu-BRU-3081 | 100 ± 0 | -Bpa4-Ψ(CH₂N)-Pro | 0.001 | 0.30 | Yes |
| BRU-2477 | 11.9 ± 2.4 | -Tyr-Pro | 0.9 | 0.84 | |
| (unstabilized | | | | | |
| parent) | | | | | |

“Metabolically Stabilized” Compounds of Form
pGlu-His-Trp-Ser-His-Darg-Trp-AA⁸-AA⁹-azaGly-NH₂

| | | | | | |
|---|---|---|---|---|---|
| BRU-3123 | 100 ± 0 | -Tyr-Ampc4- | <0.001 | 0.99 (n = 2) | Yes |
| BRU-3124 | 38.7 ± 5.9 | -Tyr-Thz- | nd | 2.49 (n = 1) | Yes |
| BRU-3122 | 100 ± 0 | -Tyr-Ψ(CH₂N)-Pro | <0.001 | 3.13 (n = 2) | Yes |

*All the data were compared to those of Lu-BRU-2813 or unstabilized peptide (student test with 2-tailed distribution and 2-sample equal variance).
nd = not determined (n = 1)

### Direct Binding and Internalization/Efflux Studies

As indicated earlier herein, various of the synthesized LHRH-II analogs of the present invention were also subjected to analysis of binding efficacy via measurement of direct binding of the radioactively labeled (with ¹⁷⁷Lu) peptides to EFO-27 ovarian cancer cells. In addition, measurements were made of the internalization and efflux of the radiolabeled peptides following binding to the cells. The assay methods were described previously herein. The results are described below.

As set forth earlier herein, it was shown that DO3A10CM-conjugated LHRH II analogs compete with ¹²⁵I-LHRH II ([¹²⁵I-Tyr⁸]BRU-2477) for binding to ovarian cancer (EFO27) cells at a range of 0.1-10 μM concentrations. To determine the relative percent of direct binding/uptake by these cells, many of these DO3A10CM-analogs were labeled with ¹⁷⁷Lu and their direct total and non-specific binding determined at a single concentration. FIG. 18 shows the total and the non-specific binding of various ¹⁷⁷Lu-LHRH II analogs to EFO-27 cells. As can be noted, most of the binding is specific and the nonspecific binding (NSB) in all these cases amounted to ≦10% of the total uptake.

Among the thirty ¹⁷⁷Lu-LHRH analogs tested, the top three binders were Lu-BRU-2968, Lu-BRU-2813 and Lu-BRU-2666, with an uptake of 23.5, 18.8 and 13.3% respectively. All three top binders have in common a highly lipophilic aromatic amino acid such as “Dnal2” at position 1 and “Bpa4” at position 8 and a basic amino acid (His or Arg) at position 2. Substituting a more basic amino acid, Arg (BRU-2813 or BRU-2968), for His (as in BRU-2666) was seen to increase the binding.

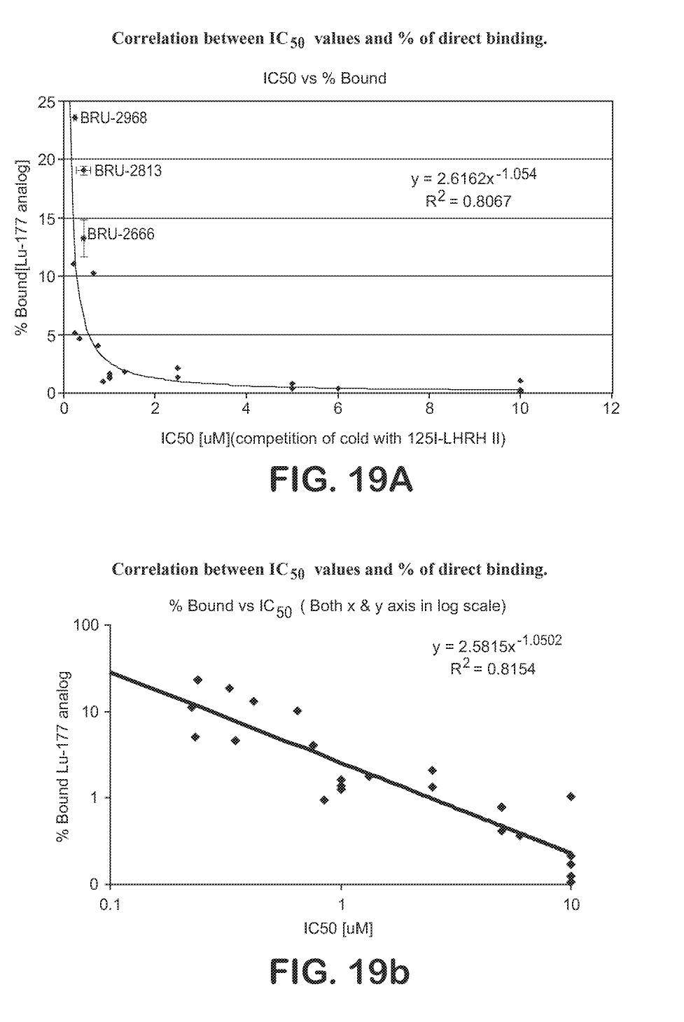

The direct binding results for the ¹⁷⁷Lu complexes were compared with the IC₅₀ values obtained from competition binding of the unlabeled analogs with ¹²⁵I-LHRH-II. As shown in FIGS. 19 a and b, a direct correlation between the competition efficiency (IC₅₀ values) of cold LHRH II analogs and the direct binding (% uptake) of ¹⁷⁷Lu-LHRH II analogs was observed, the correlation being that the lower the IC₅₀ values, the higher the % uptake of the ¹⁷⁷Lu complexes by the EFO-27 cells.

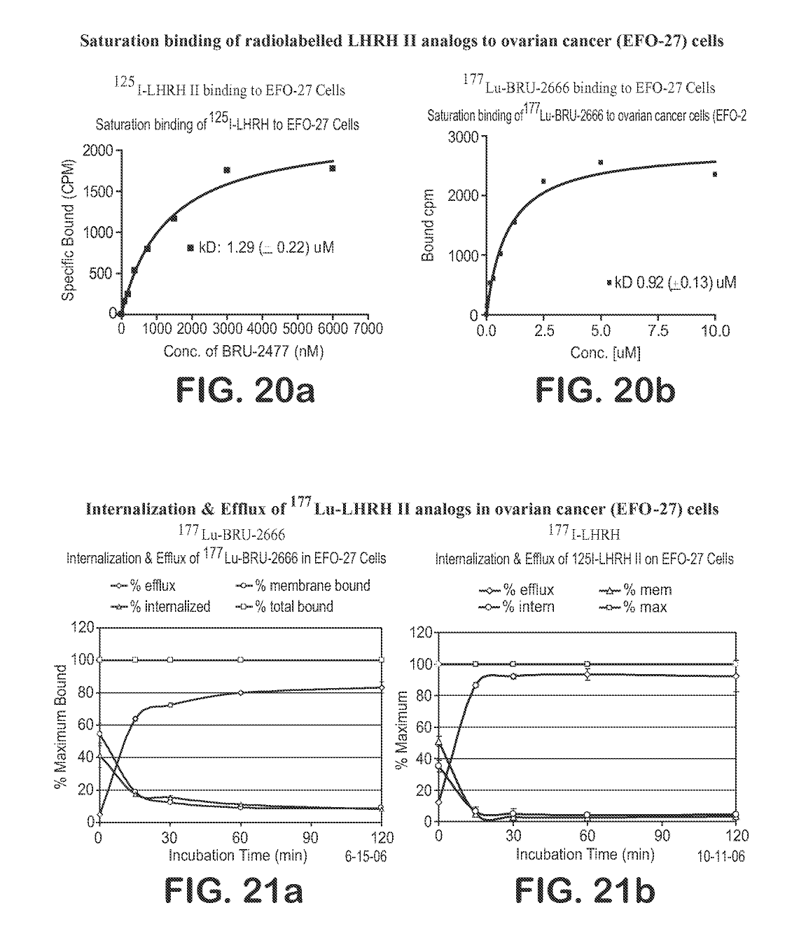

Saturation binding of ¹²⁵I-LHRH II and the ¹⁷⁷Lu-labeled LHRH II analog ¹⁷⁷Lu-BRU-2666 to EFO-27 cells was carried out to determine the binding affinity, binding capacity (Bₘₐₓ) and receptor numbers. The data were analyzed for a single binding site using Prizm software. As shown in FIGS. 20a and 20b, both ¹²⁵I-LHRH II and ¹⁷⁷Lu-BRU-2666 showed a similar affinity to EFO-27 cells, the kD values being 1.29 (±0.22) μM and 0.92(+0.13) μM respectively.

TABLE 23
Binding Data of Radio-Labeled LHRH II Analogs to Ovarian
Cancer (EFO-27) Cells

| | | Max. binding (Bₘₐₓ) | Receptors/cell |
|---|---|---|---|
| Compound | kD (μM) | (pmoles/10⁶ cells) | (×10⁶) |
| ¹⁷⁷Lu-BRU-2666 | 0.92 (±0.13) | 183 | 110 |
| ¹²⁵I[Tyr]-LHRH II | 1.29 (±0.22) | 141 | 85 |

Based on a single binding site in EFO-27 cells for ¹⁷⁷Lu-BRU-2666, the binding capacity (Bₘₐₓ) was determined to be 183 pmoles/million cells and the receptor numbers 110×10⁶/cell. For ¹²⁵I[Tyr]-LHRH II, the Bₘₐₓ was 141 pmoles/million cells and the receptors 85×10⁶/cell (Table 23).

Table 24 provides a side-by-side summary comparison of the results for peptides tested both in the competitive-binding and the direct-binding assays.

TABLE 24
Direct Binding/Uptake (%) of ¹⁷⁷Lu-LHRH II to EFO-27 Cells

| | | | ¹²⁵I-LHRH II | | ¹⁷⁷Lu-LHRH II | | |
|---|---|---|---|---|---|---|---|
| | | | Competition | | Direct binding | | |
| | BRU | | | st | | st | |
| # | No. | LHRH II analogs (X = DO3A10CM) | EC₅₀ [μM] | dev | % Bound | dev | N |
| 1 | 2477 | pGlu¹-His-Trp-Ser⁴-His-Darg⁶-Trp-Tyr⁸-Pro-azaG¹⁰-NH₂ | 0.84 | 0.28 | — | — | — |
| 2 | 2440 | X-Sar-His-Trp-Ser-His-Darg-Trp-Tyr-Pro-azaG-NH₂ | >10 | 0.00 | 0.21 | | 1 |
| 3 | 2443 | X-Sar-His-Trp-Ser-His-Darg-Trp-Bpa4-Pro-azaG-NH₂ | 0.95 | 0.20 | 1.8 | | 1 |
| 4 | 2613 | X-Gly-Sar-Dcfe4-Dnal1-Ser-Metyr-Dlys(Nic)-Leu- | >10 | 0.00 | 0.17 | | 1 |
| | | Lys(isp)-Pro-Dala-NH₂ | | | | | |
| 5 | 2624 | X-Sar-His-Trp-Ser-His-Darg-Trp-Nal2-Pro-azaG-NH₂ | 2.01 | 1.44 | 4.03 | | 1 |
| 6 | 2642 | X-Gly-Dnal2-Dcfe4-Dpal3-Ser-Metyr-Dlys(Nic)-Leu- | >10 | 0.00 | 0.105 | | 1 |
| | | Lys(isp)-Hypt4-Dala-NH₂ | | | | | |
| 7 | 2644 | X-Gly-Dnal2-Dcfe4-Dpal3-Ser-His-Darg-Trp-Tyr-Pro- | 2.91 | 0.07 | 2.13 | | 1 |
| | | Dala-NH₂ | | | | | |
| 8 | 2666 | X-Dnal2-His-Trp-Ser-His-Darg-Trp-Bpa4-Pro-azaG-NH₂ | 0.39 | 0.06 | 13.25 | 1.56 | 4 |
| 9 | 2696 | X-Gly-Abz4-Sar-His-Trp-Ser-His-Darg-Trp-Bpa4-Pro- | 0.44 | 0.22 | 0.94 | | 1 |
| | | azaG-NH₂ | | | | | |
| 10 | 2721 | X-Sar-His-Trp-Ser-His-Darg-Trp-Dip-Pro-azaG-NH₂ | 4.55 | 0.72 | 0.79 | | 1 |
| 11 | 2722 | X-Sar-His-Trp-Ser-His-Darg-Trp-F5fe-Pro-azaG-NH₂ | 4.62 | 1.91 | 0.36 | | 1 |
| 12 | 2725 | X-Sar-His-Trp-Ser-His-Darg-Trp-Cfe4-Pro-azaG-NH₂ | 2.26 | 1.21 | 0.41 | | 1 |
| 13 | 2736 | X-Meala-His-Trp-Ser-His-Darg-Trp-Bpa4-Pro-azaG-NH₂ | 1.42 | 0.40 | 1.62 | | 1 |
| 14 | 2738 | X-Sar-Phe-Trp-Ser-His-Darg-Trp-Bpa4-Pro-azaG-NH₂ | 1.22 | 0.22 | 1.41 | | 1 |
| 15 | 2741 | X-Sar-His(1-Me)-Trp-Ser-His-Darg-Trp-Bpa4-Pro-azaG- | 2.99 | 1.28 | 1.35 | | 1 |
| | | NH₂ | | | | | |
| 16 | 2742 | X-Sar-His-Trp-Ser-His-Darg-Trp-Tha-Pro-azaG-NH₂ | >10 | 0.00 | 0.125 | | 1 |
| 17 | 2756 | X-Sar-Amfe4-Trp-Ser-His-Darg-Trp-Bpa4-Pro-azaG-NH₂ | 0.28 | 0.10 | 5.17 | | 1 |
| 18 | 2757 | X-Damfe4-His-Trp-Ser-His-Darg-Trp-Bpa4-Pro-azaG-NH₂ | 0.26 | 0.04 | 11.08 | | 1 |
| 19 | 2766 | X-Gly-Dnal1-His-Trp-Ser-His-Darg-Trp-Bpa4-Pro-azaG- | 0.62 | 0.00 | 10.29 | | 1 |
| | | NH₂ | | | | | |
| 20 | 2767 | X-Sar-His-Trp-Ser-His-Darg-Trp-azaG-NH₂ | >10 | 0.0 | 1.06 | | 1 |
| 21 | 2792 | X-Gly-Bzgly-His-Trp-Ser-His-Darg-Trp-Bpa4-Pro-azaG- | 1.80 | 1.12 | 1.28 | | 1 |
| | | NH₂ | | | | | |
| 22 | 2797 | X-Gly-Hpgly-His-Trp-Ser-His-Darg-Trp-Bpa4-Pro-azaG- | 0.45 | 0.21 | 4.64 | | 1 |
| | | NH₂ | | | | | |
| 23 | 2810 | X-Dafe4-His-Trp-Ser-His-Darg-Trp-Bpa4-Pro-azaG-NH₂ | 0.98 | — | 2.40 | | 1 |
| 24 | 2812 | X-Dnal2-Afe4-Trp-Ser-His-Darg-Trp-Bpa4-Pro-azaG-NH₂ | 3.69 | — | 5.80 | | 1 |
| 25 | 2813 | X-Dnal2-Arg-Trp-Ser-His-Darg-Trp-Bpa4-Pro-azaG-NH₂ | 0.3 | — | 18.80 | | 1 |
| 26 | 2823 | X-Sar-His-Trp-Ser-His-Darg-Trp-Thy-Pro-azaG-NH₂ | 1.61 | 0.23 | 1.00 | | 1 |
| 27 | 2869 | X-Dnal1-His-Trp-Ser-His-Darg-Trp-Bpa4-Pro-azaG-NH₂ | 0.36 | — | 9.20 | | 1 |
| 28 | 2894 | X-Dphe-His-Trp-Ser-His-Darg-Trp-Bpa4-Pro-azaG-NH₂ | 2.0 | — | 3.40 | | 1 |
| 29 | 2896 | X-Adoa-Adoa-Sar-His-Trp-Ser-His-Darg-Trp-Bpa4-Pro- | 3.2 | — | 0.70 | | 1 |
| | | azaG-NH₂ | | | | | |
| 30 | 2968 | X-Dnal2-Arg-Trp-Ser-His-Darg-Trp-Bpa4-Pro-Dal5o3pt | 0.24 | 0.03 | 23.5 | | 1 |

### Internalization and Efflux Studies

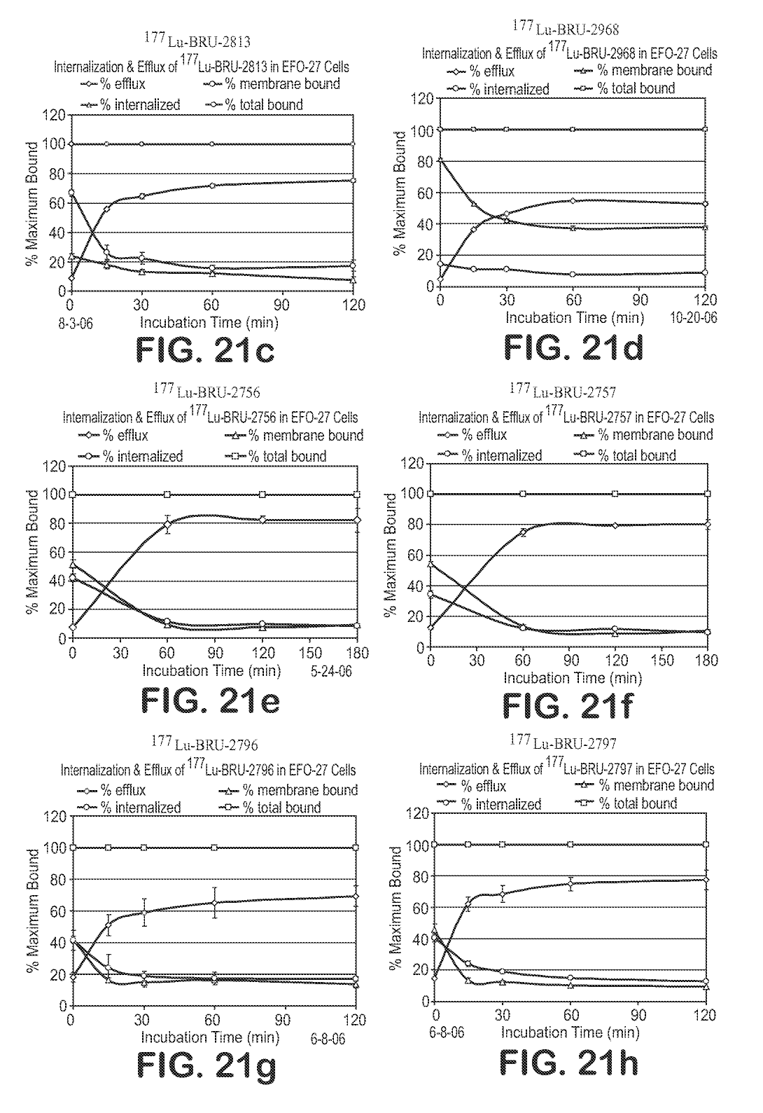

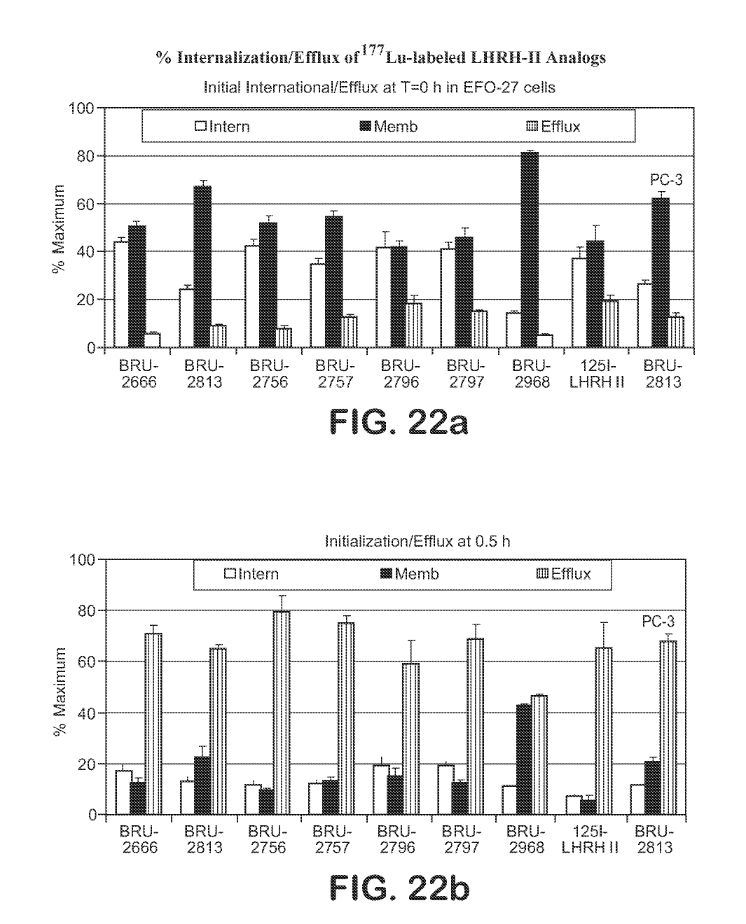

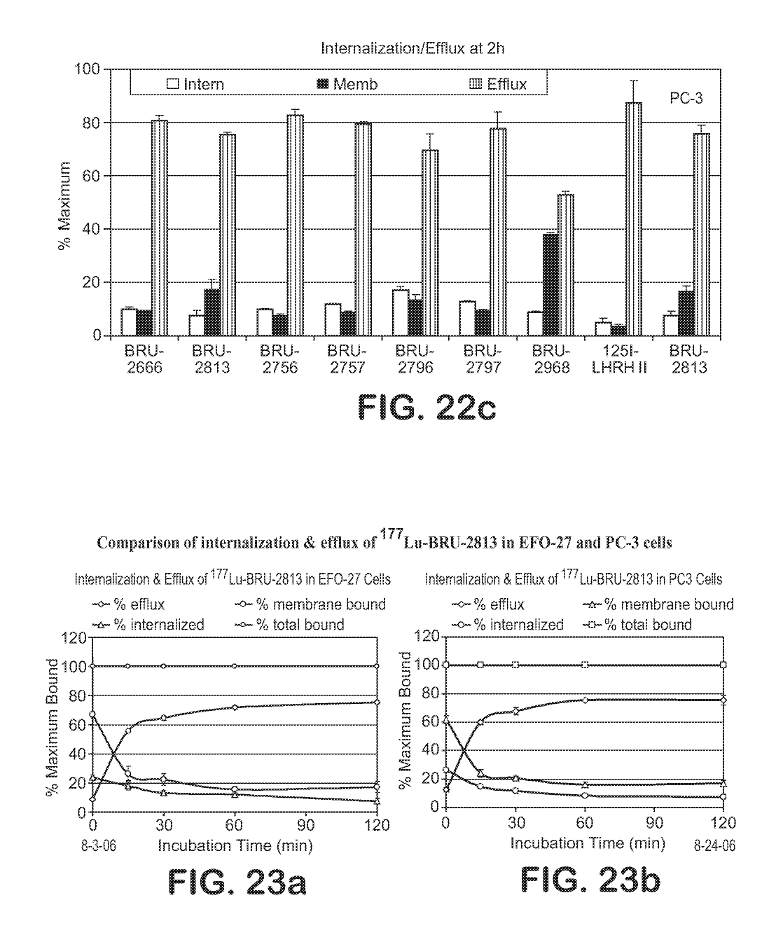

The internalization and efflux of several Lu-177 labeled LHRH II analogs in EFO-27 cells have been investigated. Basically, following pre-incubation of cells with ¹⁷⁷Lu-LHRH II samples, the binding buffer containing the ¹⁷⁷Lu was replaced with fresh media without complex. The extent of initial internalization (T=0), the amount of radioactive material that remained internalized and the efflux at 0-120 min were determined. The results are shown in FIGS. 21a-f and 22a-c and in Table 25.

TABLE 25
Internalization and Efflux of ¹⁷⁷Lu-LHRH II Analogs in EFO-27 Cells

| | | | | | Cell associated | | | Cell associated | | |
|---|---|---|---|---|---|---|---|---|---|---|
| ¹⁷⁷Lu-LHRH II | Sp | | | | activity at 0 h | | | activity at 2 h | | |
| (Formulated unless | activity | added/well | Bound | Bound | % | % | % | % | % | % |
| otherwise specified) | Ci/μmole | fmoles | fmoles | % | Intern | Memb | Efflux | Intern | Memb | Efflux |
| BRU-2666 | 1.10 | 205 | 25.0 | 12.20 | 43.6 | 50.3 | 5.5 | 10.0 | 9.5 | 80.6 |
| BRU-2756 | 1.12 | 200 | 10.2 | 5.10 | 42.0 | 51.5 | 7.7 | 9.9 | 7.4 | 82.5 |
| BRU-2757 | 1.12 | 200 | 19.0 | 9.50 | 34.5 | 54.3 | 12.4 | 11.7 | 8.8 | 79.5 |
| BRU-2796 (HPLC | 3.37 | 78 | 0.93 | 1.20 | 41.5 | 41.8 | 18.2 | 17.1 | 13.5 | 69.4 |
| purified material) | | | | | | | | | | |
| BRU-2797 | 1.11 | 203 | 4.1 | 2.02 | 40.7 | 45.8 | 14.8 | 12.8 | 9.6 | 77.6 |
| BRU-2813 | 1.10 | 212 | 40.5 | 19.10 | 24.1 | 66.8 | 8.8 | 7.4 | 17.3 | 75.3 |
| BRU-2968 | 0.772 | 292 | 68.8 | 23.59 | 14.1 | 80.8 | 4.9 | 9.0 | 38.1 | 52.9 |
| BRU-2813 (PC-3 cells) | 1.14 | 230 | 44.3 | 19.26 | 26.2 | 61.9 | 12.5 | 7.5 | 16.8 | 75.7 |
| ¹²⁵I-LHRH II | 1.68 | 156 | 1.87 | 1.20 | 36.7 | 44.6 | 19 | 4.9 | 3.5 | 87.1 |

Intern = Internalized;
Memb = Membrane bound

The initial internalization of ¹²⁵I[Tyr]-LHRH II in EFO-27 cells was found to be 35.6% of the total bound radioactivity while that of ¹⁷⁷Lu-LHRH II analogs ranged from 14-44%. Interestingly, in all cases, a high percentage, 40-80% of the total bound, was found to be on the cell surface. After changing to fresh media, in all cases efflux of radioactivity from the cells was observed. Most of the cell-associated radioactivity was washed out (60-87% efflux) into medium in less than 2 h. The high-binding analogs such as ¹⁷⁷Lu-BRU-2813 (19% uptake) and ¹⁷⁷Lu-BRU-2968 (23.5% uptake) show a lower percentage of internalization (14-24%) than the relatively low-binding analogs such as BRU-2796 (1.2% uptake) and BRU-2797 (2.0) which showed about 41% internalization. ¹⁷⁷Lu-BRU-2968, a top binder with 23.5% uptake, showed only 14% internalization but a high surface binding (80%).

To determine whether prostate cancer cells (PC-3) behave differently from EFO-27 cells, internalization and efflux studies were carried out with ¹⁷⁷Lu-BRU-2813 using both cell lines. As shown in FIG. 23, both EFO-27 and PC-3 cell lines showed low initial internalization (27% and 25% of cell-associated counts, respectively). In both cell lines, the major cell-associated activity was found to be on the cell surface (60-65% of the total bound); and both cells showed a rapid efflux (75-80%) in 1 h. Thus, both PC-3 (prostate cancer) and EFO-27 (ovarian cancer) cell lines showed a similar internalization and efflux pattern for ¹⁷⁷Lu-BRU-2813.

In summary, the direct-binding and internalization/efflux studies showed that:
1) Direct binding/uptake of ¹⁷⁷Lu-labeled LHRH-II analogs to EFO-27 cells correlates well with the IC₅₀ data generated by competition of their cold analogs with ¹²⁵I-LHRH-II, the correlation being that the lower the IC₅₀, the higher the % binding/uptake of ¹⁷⁷Lu-LHRH-II.
2) ¹²⁵I-LHRH-II and ¹⁷⁷Lu-BRU-2666 bind to ovarian cancer cells (EFO-27) in a saturable manner, the kDs being 1.29 μM and 0.92 μM, respectively.
3) ¹⁷⁷Lu-LHRH II analogs showed low-to-moderate internalization (14-44%) in EFO-27 and PC-3 cells, and exhibited an efflux of >75% in 2 h.
4) ¹⁷⁷Lu-BRU-2968, a top binder, showed lowest internalization (14%) and high surface binding (80%).

TABLE 26
Summary Table: Characterization Data for LHRH-II Peptides

| | | | | | | |
|---|---|---|---|---|---|---|
| | | | HPLC Data (System, | | Mass Spectral Data | |
| Seq. ID | BRU # | Sequence | t_{R}, min) | M. Wt | (Mode: Ions) | EC₅₀ μM |

Underivatized LHRH Peptides and LHRH Peptides with Chelator on N-terminus

| | | | | | | |
|---|---|---|---|---|---|---|
| Seq005 | BRU-2441 | -Sar-His-Trp-Ser-His-Darg-Trp-Bpa4-Pro-azaGly-NH₂ | B, 6.84 | 1385 | Neg. ion: [M + 2TFA]: | 0.14 |
| | | | | | 1610.4, [M + TFA]: | |
| | | | | | 1496.4, [M − H]: 1382.4 | |
| Seq209 | BRU-3100 | DO3A10CM-Da48oa-Dnal2-Arg-Trp-Ser-His-Darg-Trp-Bpa4-Pro- | D, 7.38 | 2146 | Pos. ion: [2M + 3H]/3: | 0.14 |
| | | Da15o3pt | | | 1431.0; [M + Na + H]/2: | |
| | | | | | 1084.4; [M + 2H]/2: | |
| | | | | | 1073.4; [M + 3H]/3: | |
| | | | | | 716.0; [M + 4H]/4: 537.2 | |
| Seq042 | BRU-2734 | Mephe-His-Trp-Ser-His-Darg-Trp-Bpa4-Pro-azaGly-NH₂ | D, 7.36 | 1475 | Pos. ion: [M + Na]: | 0.15 |
| | | | | | 1497.6; [M + H]: 1474.6; | |
| | | | | | [M + Na + H]/2: 748.9; | |
| | | | | | [M + 2H]/2: 738.0; | |
| | | | | | [M + 3H]/3: 492.4 | |
| Seq221 | BRU-3115 | DO3A10CM-Dnal2-Arg-Trp-Amfe4-His-Darg-Trp-Bpa4-Pro-azaGly-NH₂ | D, 6.56 | 2005 | Pos. ion: [2M + 3H]/3: | 0.15 |
| | | | | | 1337.6; [M + 2H]/2: | |
| | | | | | 1003.4; [M + 3H]/3: | |
| | | | | | 669.2; [M + 4H]/4: 502.2. | |
| Seq199 | BRU-3072 | DO3A10CM-Dnal2-Arg-Trp-Ser-His-Darg-Trp-Bpa4-Thz-azaGly-NH₂ | D, 7.72 | 1875 | Pos. ion: [M + H]: | 0.16 |
| | | | | | 1874.8; [M + Na + H]/2: | |
| | | | | | 947.8; [M + 2H]/2: 936.8; | |
| | | | | | [M + 3H]/3: 624.8 | |
| Seq126 | BRU-2964 | DO3A10CM-Dnal2-Arg-Trp-Met-His-Darg-Trp-Bpa4-Pro-azaGly-NH₂ | D, 7.92 | 1960 | Pos. ion: [M + H]: | 0.22 |
| | | | | | 1961.3; [M + 2H]/2: | |
| | | | | | 980.8; [M + 3H]/3: 654.4 | |
| Seq158 | BRU-3007 | Sar-Arg-Trp-Ser-His-Darg-Trp-Bpa4-Pro-Da1503pt | F, 2.72 | 1433 | Pos. ion: [M + H]: | 0.22 |
| | | | | | 1433.6; [M + 2H]/2: | |
| | | | | | 717.0; [M + 3H]/3: 478.4 | |
| Seq211 | BRU-3105 | DO3A10CM-Dnal2-Arg-Nal2-Ser-His-Darg-Trp-Bpa4-Pro-azaGly-NH₂ | D, 8.26 | 1927 | Pos. ion: [M + H]: | 0.22 |
| | | | | | 1927.8; [2M + 3H]/3: | |
| | | | | | 1285.6; [M + Na + H]/2: | |
| | | | | | 975.4; [M + 2H]/2: 964.4; | |
| | | | | | [M + 3H]/3: 643.2 | |
| Seq129 | BRU-2968 | DO3A10CM-Dnal2-Arg-Trp-Ser-His-Darg-Trp-Bpa4-Pro-Da15o3pt | F, 3.49 | 1945 | Pos. ion: [M + 2H]/2: | 0.24 |
| | | | | | 973.2; [M + Na + H]/2: | |
| | | | | | 984.2; [M + 3H]/3: 649.3 | |
| Seq130 | BRU-2969 | DO3A10CM-Dnal2-Arg-Trp-Ser-His-Darg-Trp-Bpa4-Pro-Gly-Arg-NH₂ | F, 4.78 | 2071 | Pos. ion: | 0.24 |
| | | | | | [M + TFA + 2H]/2: | |
| | | | | | 1092.6; | |
| | | | | | [M + TFA + Na + H]/2: | |
| | | | | | 1103.6; [M + Na + 2H]/3: | |
| | | | | | 698.6; [M + 3H]/3: 691.4 | |
| Seq195 | BRU-3068 | DO3A10CM-Gly-Dtpi-Arg-Trp-Ser-His-Darg-Trp-Bpa4-Pro-azaGly-NH₂ | D, 7.69 | 1974 | Pos. ion: [M + H]: | 0.24 |
| | | | | | 1974.8; [M + Na + H]/2: | |
| | | | | | 998.8 [M + 2H]/2: 987.8; | |
| | | | | | [M + 3H]/3: 658.8 | |
| Seq003 | BRU-2439 | Sar-His-Trp-Ser-His-Darg-Trp-Tyr-Pro-azaGly-NH₂ | A, 7.33 | 1295 | Neg. ion: [M + 2TFA]: | 0.25 |
| | | | | | 1522.4, [M + TFA]: | |
| | | | | | 1408.4, [M − H]: 1294.4 | |
| Seq095 | BRU-2839 | Ac-Amfe4-His-Trp-Ser-His-Darg-Trp-Bpa4-Pro-azaGly-NH₂ | D, 6.55 | 1532 | Pos. ion: [M + H]: | 0.25 |
| | | | | | 1532.5; [M + Na + H]/2: | |
| | | | | | 777.4; [M + 2H]/2: 766.3; | |
| | | | | | [M + 3H]/3: 511.5 | |
| Seq121 | BRU-2959 | DO3A10CM-Dnal2-Arg-Trp-Ser-Arg-Darg-Trp-Bpa4-Pro-azaGly-NH₂ | D, 7.55 | 1935 | Pos. ion: [M + H + Na]/2: | 0.25 |
| | | | | | 979.2, [M + 2H]/2: 968.2, | |
| | | | | | [M + 3H]/3: 645.8 | |
| Seq210 | BRU-3104 | DO3A10CM-Dnal2-Arg-Nal1-Ser-His-Darg-Trp-Bpa4-Pro-azaGly-NH₂ | D, 8.02 | 1927 | Pos. ion: [M + H]: | 0.25 |
| | | | | | 1927.8; [2M + 3H]/3: | |
| | | | | | 1285.4; [M + Na + H]/2: | |
| | | | | | 975.4; [M + 2H]/2: 964.4; | |
| | | | | | [M + 3H]/3: 643.2 | |
| Seq217 | BRU-3111 | DO3A10CM-Dnal2-Arg-Arg-Ser-His-Darg-Trp-Bpa4-Pro-azaGly-NH₂ | D, 8.06 | 1886 | Pos. ion: [M + H]: | 0.25 |
| | | | | | 1886.8; [2M + 3H]/3: | |
| | | | | | 1258.0; [M + 2H]/2: | |
| | | | | | 943.6; [M + 3H]/3: 629.6, | |
| | | | | | [M + 4H]/4: 472.4. | |
| Seq053 | BRU-2757 | DO3A10CM-Damfe4-His-Trp-Ser-His-Darg-Trp-Bpa4-Pro-azaGly-NH₂ | D, 6.14 | 1876 | Pos. ion: [M + Na]/2: | 0.26 |
| | | | | | 949.7; [M + 2H]/2: 938.8; | |
| | | | | | [M + Na]/3: 633.4; | |
| | | | | | [M + 3H]/3: 626.2; | |
| | | | | | [M + 4H]/4: 469.9 | |
| Seq078 | BRU-2803 | Ac-Dnal2-His-Trp-Ser-His-Darg-Trp-Bpa4-Pro-azaGly-NH₂ | D, 8.67 | 1553 | Pos. ion: [M + H]: | 0.26 |
| | | | | | 1553.5, [M + Na + H]/2: | |
| | | | | | 788.2, [M + 2H]/2: 777.0, | |
| | | | | | [M + 3H]/3: 518.5 | |
| Seq185 | BRU-3058 | DO3A10CM-Dnal2-Gufe4-Trp-Ser-His-Darg-Trp-Bpa4-Pro-azaGly-NH₂ | D, 7.12 | 1964 | Pos. ion: [M + H]: | 0.26 |
| | | | | | 1964.8; [2M + 3H]/3: | |
| | | | | | 1310.8; [M + 2H]/2: | |
| | | | | | 962.8; [M + 3H]/3: 656.6; | |
| | | | | | [M + 4H]/4: 492.0 | |
| Seq118 | BRU-2956 | DO3A10CM-Dnal2-Arg-Trp-Asn-His-Darg-Trp-Bpa4-Pro-azaGly-NH₂ | D, 7.31 | 1943 | Pos. ion: [M + H + Na]/2: | 0.27 |
| | | | | | 983.4; [M + 2H]/2: 972.2; | |
| | | | | | [M + 3H]/3: 648.8 | |
| Seq114 | BRU-2952 | DO3A10CM-Dnal2-Arg-Trp-Ser-His-Darg-Trp-Bpa4-Hypt4-azaGly-NH₂ | D, 7.23 | 1932 | Pos. ion: [M + H + Na]/2: | 0.28 |
| | | | | | 977.6; [M + 2H]/2: 966.8; | |
| | | | | | [M + 3H]/3: 696.2; | |
| | | | | | [M + 4H]/4: 644.8 | |
| Seq125 | BRU-2963 | DO3A10CM-Ambz4-Dnal2-Arg-Trp-Ser-His-Darg-Trp-Bpa4-Pro-azaGly- | D, 7.52 | 2049 | Pos. ion: [M + H]: | 0.28 |
| | | NH₂ | | | 2050.3; [M + 2H]/2: | |
| | | | | | 1025.4; [M + 3H]/3: | |
| | | | | | 684.0 | |
| Seq197 | BRU-3070 | DO3A10CM-Bip-Arg-Trp-Ser-His-Darg-Trp-Bpa4-Pro-azaGly-NH₂ | D, 7.68 | 1942 | Pos. ion: [M + H]: | 0.28 |
| | | | | | 1942.8; [M + Na + H]/2: | |
| | | | | | 982.8; [M + 2H]/2: 971.8; | |
| | | | | | [M + 3H]/3: 648.2 | |
| Seq204 | BRU-3095 | DO3A10CM-Damfe4-Damfe4-Trp-Ser-His-Darg-Trp-Bpa4-Pro-azaGly- | D, 6.63 | 1915 | Pos. ion: [M + Na]: | 0.29 |
| | | NH₂ | | | 1937.8; [M + H]: 1916.2; | |
| | | | | | [M + Na + H]/2: 969.2; | |
| | | | | | [M + 2H]/2: 958.4; | |
| | | | | | [M + 3H]/3: 639.2 | |
| Seq200 | BRU-3081 | DO3A10CM-Dnal2-Arg-Trp-Ser-His-Darg-Trp-Bpa4-psi(CH₂N)-Pro- | D, 7.15 | 1902 | Pos. ion: [M + H]: | 0.30 |
| | | azaGly-NH₂ | | | 1901.8; [M + Na + H]/2: | |
| | | | | | 962.8; [M + 2H]/2: 951.8; | |
| | | | | | [M + 3H]/3: 634.8, | |
| | | | | | [M + 4H]/4: 476.4 | |
| Seq169 | BRU-3031 | DO3A10CM-Dnal2-Arg-Trp-Ser-His-Arg-Trp-Bpa4-Pro-azaGly-NH₂ | D, 6.45 | 1916 | Pos. ion: [M + H]: | 0.31 |
| | | | | | 1917.8; [M + Na + H]/2: | |
| | | | | | 969.8; [M + 2H]/2: 958.8; | |
| | | | | | [M + 3H]/3: 639.4 | |
| Seq177 | BRU-3050 | DO3A10CM-Arg-Arg-Trp-Ser-His-Darg-Trp-Bpa4-Pro-azaGly-NH₂ | D, 7.61 | 1875 | Pos. ion: [M + H]: | 0.31 |
| | | | | | 1875.5; [2M + 3H]/3: | |
| | | | | | 1250.8; [M + 2H]/2: | |
| | | | | | 938.4; [M + 3H]/3: 625.8; | |
| | | | | | [M + 4H]/4: 469.6 | |
| Seq198 | BRU-3071 | DO3A10CM-Dbpa4-Arg-Trp-Ser-His-Darg-Trp-Bpa4-Pro-azaGly-NH₂ | D, 7.67 | 1970 | Pos. ion: [M + H]: | 0.31 |
| | | | | | 1970.8; [M + Na + H]/2: | |
| | | | | | 996.8; [M + 2H]/2: 985.8; | |
| | | | | | [M + 3H]/3: 657.6 | |
| Seq180 | BRU-3053 | DO3A10CM-Gly-Tpi-Arg-Trp-Ser-His-Darg-Trp-Bpa4-Pro-azaGly-NH₂ | D, 6.88 | 1974 | Pos. ion: [M + H]: | 0.32 |
| | | | | | 1974.8; [2M + 3H]/3: | |
| | | | | | 1316.6; [M + 2H]/2: | |
| | | | | | 967.8; [M + 3H]/3: 658.8 | |
| Seq189 | BRU-3062 | DO3A10CM-Dnal2-Arg-Trp-Ser-His-Darg-Trp-Bpa4-Ampc4-azaGly-NH₂ | D, 7.85 | 1931 | Pos. ion: [M + H]: | 0.32 |
| | | | | | 1931.8; [M + Na + H]/2: | |
| | | | | | 976.8; [M + 2H]/2: 965.8; | |
| | | | | | [M + 3H]/3: 644.2 | |
| Seq084 | BRU-2813 | DO3A10CM-Dnal2-Arg-Trp-Ser-His-Darg-Trp-Bpa4-Pro-azaGly-NH₂ | D, 7.73 | 1916 | Pos. ion; [M + Na]: | 0.33 |
| | | | | | 1938.8, [M + 2H]/2: | |
| | | | | | 958.8; 646.8; [M + 3H]/3: | |
| | | | | | 639.6; [M + 4H]/4: 480. | |
| Seq149 | BRU-2997 | DO3A10CM-Damfe4-Arg-Trp-Ser-His-Darg-Trp-Bpa4-Pro-azaGly-NH₂ | D, 6.66 | 1895 | Pos. ion: [M + 2H]/2: | 0.33 |
| | | | | | 948.4; [M + 3H]/3: 632.6 | |
| Seq075 | BRU-2796 | DO3A10CM-Gly-Mogly-His-Trp-Ser-His-Darg-Trp-Bpa4-Pro-azaGly- | D, 6.78 | 1872 | Pos. ion: [M + Na]: | 0.34 |
| | | NH₂ | | | 1895.8, [M + Na + H]/2: | |
| | | | | | 947.8; [M + 2H]/2: 936.8; | |
| | | | | | [M + 3H]/3: 624.8; | |
| Seq187 | BRU-3060 | DO3A10CM-Dnal2-Ampg4-Trp-Ser-His-Darg-Trp-Bpa4-Pro-azaGly-NH₂ | D, 7.39 | 1942 | Pos. ion: [M + H]: | 0.34 |
| | | | | | 1942.8; [2M + 3H]/3: | |
| | | | | | 1295.6; [M + 2H]/2: | |
| | | | | | 971.8; [M + 3H]/3: 648.2 | |
| Seq123 | BRU-2961 | DO3A10CM-Dnal2-Arg-Trp-Ser-Orn-Darg-Trp-Bpa4-Pro-azaGly-NH₂ | D, 7.51 | 1893 | Pos. ion: [M + 2H]/2: | 0.35 |
| | | | | | 947.2 | |
| Seq148 | BRU-2996 | DO3A10CM-Dnal2-Arg-Trp-Ser-His-Darg-Trp-Bpa4-Pip-azaGly-NH₂ | D, 8.42 | 1930 | Pos. ion: [M + H]: | 0.35 |
| | | | | | 1930.8; [M + Na + H]/2: | |
| | | | | | 976.8; [M + 2H]/2: 965.8; | |
| | | | | | [M + 3H]/3: 644.2; | |
| | | | | | [M + 4H]/4: 483.4 | |
| Seq203 | BRU-3094 | DO3A10CM-Ampha4-Arg-Trp-Ser-His-Darg-Trp-Bpa4-Pro-azaGly-NH₂ | D, 6.28 | 1929 | Pos. ion: [2M + 3H]/3: | 0.35 |
| | | | | | 1286.8; [M + 2H]/2: | |
| | | | | | 965.4; [M + 3H]/3: 643.8; | |
| | | | | | [M + 4H]/4: 483.2 | |
| Seq082 | BRU-2811 | DO3A10CM-His-Trp-Ser-His-Darg-Trp-Bpa4-Pro-azaGly-NH₂ | D, 6.69 | 1700 | Pos. ion: [M + Na]/2: | 0.36 |
| | | | | | 861.4, [M + 2H]/2: 850.5, | |
| | | | | | [M + 3H]/3: 567.4, | |
| | | | | | [M + 4H]/4: 426.0 | |
| Seq096 | BRU-2869 | DO3A10CM-Dnal1-His-Trp-Ser-His-Darg-Trp-Bpa4-Pro-azaGly-NH₂ | D, 5.19 | 1897 | Pos. ion: [M + Na + H]/2: | 0.36 |
| | | | | | 960.4; [M + 2H]/2: 949.3; | |
| | | | | | [M + 3H]/3: 633.3; | |
| | | | | | [M + 4H]/4: 475.1 | |
| Seq176 | BRU-3049 | DO3A10CM-Dnal2-Darg-Trp-Ser-His-Darg-Trp-Bpa4-Pro-azaGly-NH₂ | D, 7.63 | 1916 | Pos. ion: [M + H]: | 0.36 |
| | | | | | 1917.8; [M + Na + H]/2: | |
| | | | | | 969.8; [M + 2H]/2: 958.8; | |
| | | | | | [M + 3H]/3: 639.6 | |
| Seq165 | BRU-3027 | DO3A10CM-Dnal2-Arg-Trp-Ser-His-Darg-Trp-Nal2-Pro-azaGly-NH₂ | D, 7.48 | 1862 | Pos. ion: [M + H]: | 0.37 |
| | | | | | 1863.8; [M + 2H]/2: | |
| | | | | | 931.8; [M + 3H]/3: 621.4 | |
| Seq205 | BRU-3096 | DO3A10CM-Dap-Dnal2-Arg-Trp-Ser-His-Darg-Trp-Bpa4-Pro-azaGly- | D, 6.76 | 2002 | Pos. ion: [2M + 3H]/3: | 0.37 |
| | | NH₂ | | | 1335.4; [M + 2H]/2: | |
| | | | | | 1001.8; [M + 3H]/3: | |
| | | | | | 668.2 | |
| Seq145 | BRU-2993 | DO3A10CM-Dnal2-Arg-Trp-Ser-His-Darg-Trp-Bpa4-Aze-azaGly-NH₂ | D, 7.61 | 1902 | Pos. ion: [M + H]: | 0.38 |
| | | | | | 1902.8; [M + Na + H]/2: | |
| | | | | | 962.8; [M + 2H]/2: 951.8; | |
| | | | | | [M + Na + 2H]/3: 634.8 | |
| Seq184 | BRU-3057 | DO3A10CM-Qua3-Arg-Trp-Ser-His-Darg-Trp-Bpa4-Pro-azaGly-NH₂ | D, 6.68 | 1917 | Pos. ion: [M + H]: | 0.38 |
| | | | | | 1918.8; [2M + 3H]/3: | |
| | | | | | 1278.8; [M + 2H]/2: | |
| | | | | | 959.4; [M + 3H]/3: 639.8; | |
| | | | | | [M + 4H]/4: 480.2 | |
| Seq196 | BRU-3069 | DO3A10CM-Thy-Arg-Trp-Ser-His-Darg-Trp-Bpa4-Pro-azaGly-NH₂ | D, 7.68 | 1974 | Pos. ion: [M + H]: | 0.38 |
| | | | | | 1974.8; [2M + 3H]/3: | |
| | | | | | 1316.6; [M + Na + H]/2: | |
| | | | | | 998.8; [M + 2H]/2: 987.8; | |
| | | | | | [M + 3H]/3: 658.8 | |
| Seq213 | BRU-3107 | DO3A10CM-Dnal2-Arg-Amfe4-Ser-His-Darg-Trp-Bpa4-Pro-azaGly-NH₂ | D, 6.58 | 1906 | Pos. ion: [M + H]: | 0.38 |
| | | | | | 1905.8; [2M + 3H]/3: | |
| | | | | | 1271.4; [M + 2H]/2: | |
| | | | | | 953.8; [M + 3H]/3: 636.2 | |
| Seq182 | BRU-3055 | DO3A10CM-Atdc2-Arg-Trp-Ser-His-Darg-Trp-Bpa4-Pro-azaGly-NH₂ | D, 10.62 | 1944 | Pos. ion: [M + H]: | 0.39 |
| | | | | | 1944.8; [M + 2H]/2: | |
| | | | | | 972.8; [M + 3H]/3: 648.8 | |
| Seq092 | BRU-2821 | Sar-His-Trp-Ser-His-Darg-Trp-Bpa4-Pro-azaGly-NH₂ | D, 6.59 | 1385 | Pos. ion: [M + H]: | 0.40 |
| | | | | | 1384.4, [M + Na]/2: | |
| | | | | | 703.8, [M + 2H]/2: 692.8, | |
| | | | | | [M + 3H]/3: 462.4 | |
| Seq122 | BRU-2960 | DO3A10CM-Dnal2-Arg-Trp-Ser-Fur3ala-Darg-Trp-Bpa4-Pro-azaGly- | D, 7.33 | 1916 | Pos. ion: [M + 2H]/2: | 0.40 |
| | | NH₂ | | | 959.0 | |
| Seq139 | BRU-2984 | DO3A10CM-Dnal2-Arg-Trp-Ser-His-Darg-Trp-Bpa4-Pro-Gua | F, 3.90 | 1900 | Pos ion: [M + 2H]/2: | 0.40 |
| | | | | | 950.8; [M + 3H]/3: 634.2 | |
| Seq117 | BRU-2955 | DO3A10CM-Dnal2-Arg-Trp-Dap-His-Darg-Trp-Bpa4-Pro-azaGly-NH₂ | D, 7.52 | 1915 | Pos. ion: [M + H + Na]/2: | 0.41 |
| | | | | | 969.2; [M + 2H]/2: 958.4; | |
| | | | | | [M + 3H]/3: 639.2 | |
| Seq147 | BRU-2995 | DO3A10CM-Dnal2-Arg-Trp-Ser-His-Darg-Trp-Bpa4-Ampt4-azaGly-NH₂ | D, 7.70 | 1931 | Pos. ion: [M + Na + H]/2: | 0.41 |
| | | | | | 977.4; [M + 2H]/2: 966.4; | |
| | | | | | [M + Na + 2H]/3: 651.8; | |
| | | | | | [M + 3H]/3: 644.6; | |
| | | | | | [M + 4H]/4: 483.6 | |
| Seq186 | BRU-3059 | DO3A10CM-Dnal2-Ampa4-Trp-Ser-His-Darg-Trp-Bpa4-Pro-azaGly-NH₂ | D, 6.64 | 1959 | Pos. ion: [M + 2Na]/2: | 0.41 |
| | | | | | 997.8; [M + 2H]/2: 977.4; | |
| | | | | | [2M + 2Na + H]/3: 1319.3 | |
| Seq207 | BRU 3098 | Lys(DO3A10CM)-Dnal2-Arg-Trp-Ser-His-Darg-Trp-Bpa4-Pro-azaGly- | D, 6.96 | 2044 | Pos. ion: [2M + 3H]/3: | 0.41 |
| | | NH₂ | | | 1363.6; [M + 2H]/2: | |
| | | | | | 1022.8; [M + 3H]/3: | |
| | | | | | 682.2; [M + 4H]/4: 512.0 | |
| Seq093 | BRU-2822 | pGlu-His-Trp-Ser-His-Darg-Trp-Bpa4-Pro-azaGly-NH₂ | D, 6.95 | 1425 | Pos. ion: [M + Na]: | 0.42 |
| | | | | | 1446.4; [M + H]: 1424.7; | |
| | | | | | [M + Na + H]/2: 724.3; | |
| | | | | | [M + 2H]/2: 712.9; | |
| | | | | | [M + 3H]/3: 475.8 | |
| Seq157 | BRU-3006 | DO3A10CM-Dnal2-Arg-Trp-Ser-His-Darg-Trp-Bpa4-Pro-Ap | F, 5.36 | 1934 | Pos. ion: [M + H]: | 0.42 |
| | | | | | 1934.8; [M + 2H]/2: | |
| | | | | | 967.8; [M + 3H]/3: 645.6 | |
| Seq181 | BRU-3054 | DO3A10CM-Dtyr-Arg-Trp-Ser-His-Darg-Trp-Bpa4-Pro-azaGly-NH₂ | D, 6.97 | 1884 | Pos. ion: [M + H]: | 0.43 |
| | | | | | 1881.8; [2M + 3H]/3: | |
| | | | | | 1255.4; [M + 2H]/2: | |
| | | | | | 941.8; [M + 3H]/3: 628.2 | |
| Seq212 | BRU-3106 | DO3A10CM-Dnal2-Arg-Phe-Ser-His-Darg-Trp-Bpa4-Pro-azaGly-NH₂ | D, 6.83 | 1877 | Pos. ion: [M + H]: | 0.43 |
| | | | | | 1876.8; [M + Na + H]/2: | |
| | | | | | 950.2; [M + 2H]/2: 939.4; | |
| | | | | | [M + 3H]/3: 626.6 | |
| Seq025 | BRU-2696 | DO3A10CM-Gly-Abz4-Sar-His-Trp-Ser-His-Darg-Trp-Bpa4-Pro-azaGly- | D, 6.79 | 1947 | Pos. ion: [M + Na]/2: | 0.44 |
| | | NH₂ | | | 985.9, [M + 2H]/2: 974.3, | |
| | | | | | [M + Na]/3: 657.1, | |
| | | | | | [M + 3H]/3: 650.0, | |
| | | | | | [M + 4H]/4: 487.6 | |
| Seq128 | BRU-2967 | DO3A10CM-Dnal2-Arg-Trp-Ser-His-Darg-Trp-Bpa4-Pro-Az34m3buo- | F, 5.19 | 1944 | Neg. ion: [M − 2H + Na]: | 0.44 |
| | | NH₂ | | | 984.2; [M − 2H]/2: 973.2; | |
| | | | | | [M + Na − 4H]/3: 956.7; | |
| | | | | | [M − 3H]/3: 649.3 | |
| Seq183 | BRU-3056 | DO3A10CM-Apsp-Arg-Trp-Ser-His-Darg-Trp-Bpa4-Pro-azaGly-NH₂ | D, 7.39 | 1945 | Pos. ion: [M + H]: | 0.44 |
| | | | | | 1945.8; [2M + 3H]/3: | |
| | | | | | 1297.4; [M + 2H]/2: | |
| | | | | | 973.2; [M + 3H]/3: 649.2 | |
| Seq208 | BRU-3099 | DO3A10CM-Dlys-Dnal2-Arg-Trp-Ser-His-Darg-Trp-Bpa4-Pro-azaGly- | D, 7.24 | 2044 | Pos. ion: [2M + 3H]/3: | 0.44 |
| | | NH₂ | | | 1363.6; [M + Na + H]: | |
| | | | | | 1033.8; [M + 2H]/2: | |
| | | | | | 1022.8; [M + Na + 2H]/3: | |
| | | | | | 689.6; [M + 3H]/3: 682.2 | |
| Seq076 | BRU-2797 | DO3A10CM-Gly-Hpgly-His-Trp-Ser-His-Darg-Trp-Bpa4-Pro-azaGly- | D, 6.91 | 1934 | Pos. ion: [M + Na]/2: | 0.45 |
| | | NH₂ | | | 978.4; [M + 2H]/2: 967.9; | |
| | | | | | [M + 3H]/3: 645.5; | |
| | | | | | [M + 4H]/4: 484.5 | |
| Seq138 | BRU-2983 | DO3A10CM-Dnal2-Arg-Trp-Ser-His-Darg-Trp-Bpa4-Pro-Pheol | F, 5.01 | 1992 | Pos ion: [M + H]: 1992.8; | 0.45 |
| | | | | | [M + 2H]/2: 996.6; | |
| | | | | | [M + 3H]/3: 664.9 | |
| Seq160 | BRU-3020 | DO3A10CM Dnal2-Arg-Trp-Ser-His-Darg-Trp-Bpa4-Pro-Mo2abn | F, 5.31 | 1978 | Pos. ion: [M + 2H]/2: | 0.45 |
| | | | | | 989.8, [M + 3H]/3: 660.2 | |
| Seq206 | BRU-3097 | DO3A10CM-Lys-Dnal2-Arg-Trp-Ser-His-Darg-Trp-Bpa4-Pro-azaGly- | D, 7.02 | 2044 | Pos. ion: [2M + 3H]/3: | 0.45 |
| | | NH₂ | | | 1363.6; [M + Na + H]/2: | |
| | | | | | 1033.6; [M + 2H]/2: | |
| | | | | | 1022.8; [M + 2H + Na]/3: | |
| | | | | | 689.8; [M + 3H]/3: 682.2 | |
| Seq140 | BRU-2985 | DO3A10CM-Dnal2-Arg-Trp-Ser-His-Darg-Trp-Bpa4-Pro-Algua5o3pt | F, 3.83 | 1987 | Pos. ion: [M + 2H]/2: | 0.46 |
| | | | | | 994.4; [M + 3H]/3: 663.2; | |
| Seq013 | BRU-2666 | DO3A10CM-Dnal2-His-Trp-Ser-His-Darg-Trp-Bpa4-Pro-azaGly-NH₂ | D, 8.16 | 1897 | Pos. ion: [M + Na + H]/2: | 0.47 |
| | | | | | 960.4; [M + 2H]/2: 949.3; | |
| | | | | | [M + Na]/3: 640.6; | |
| | | | | | [M + 3H]/3: 633.3; | |
| | | | | | [M + 4H]/4: 475.1 | |
| Seq124 | BRU-2962 | DO3A10CM-Gly-Dnal2-Arg-Trp-Ser-His-Darg-Trp-Bpa4-Pro-azaGly- | D, 7.52 | 1973 | Pos. ion: [M + H + Na]/2: | 0.47 |
| | | NH₂ | | | 987.4; [M + 2H]/2: | |
| | | | | | 979.8; [M + 3H]/3: 658.6 | |
| Seq163 | BRU-3025 | DO3A10CM-His-Arg-Trp-Ser-His-Darg-Trp-Bpa4-Pro-azaGly-NH₂ | D, 6.43 | 1856 | Pos. ion: [M + H]: | 0.47 |
| | | | | | 1856.8; [M + 2H]/2: | |
| | | | | | 928.8; [M + 3H]/3: 619.5 | |
| Seq190 | BRU-3063 | DO3A10CM-Datdc2-Arg-Trp-Ser-His-Darg-Trp-Bpa4-Pro-azaGly-NH₂ | D, 10.66 | 1944 | Pos. ion: [M + H]: | 0.47 |
| | | | | | 1945.8; [M + 2H]/2: | |
| | | | | | 972.8; [M + 3H]/3: 648.8 | |
| Seq132 | BRU-2971 | DO3A10CM-Dnal2-Arg-Trp-Ser-His-Darg-Trp-Bpa4-Pro-Gly-Gln-NH₂ | F, 5.05 | 2043 | Pos. ion: | 0.49 |
| | | | | | [M + TFA + 2H]/2: | |
| | | | | | 1078.4; | |
| | | | | | [M + Na + H + TFA]/2: | |
| | | | | | 1089.6; [M + 3H]/3: | |
| | | | | | 682.0 | |
| Seq103 | BRU-2876 | DO3A10CM-Gly-Ahgly-His-Trp-Ser-His-Darg-Trp-Bpa4-Pro-azaGly- | D, 4.38 | 1913 | Pos. ion: [M + Na + H]/2: | 0.50 |
| | | NH₂ | | | 968.3; [M + 2H]/2: 957.4; | |
| | | | | | [M + 3H]/3: 638.5; | |
| | | | | | [M + 4H]/4: 479.2 | |
| Seq154 | BRU-3002 | DO3A10CM-Gly-Abz4-Dnal2-Arg-Trp-Ser-His-Darg-Trp-Bpa4-Pro- | D, 8.88 | 2092 | Pos. ion: [M + 2H]/2: | 0.50 |
| | | azaGly-NH₂ | | | 1046.8; [M + Na + 2H]/3: | |
| | | | | | 705.6; [M + 3H]/3: 698.2; | |
| | | | | | [M + 4H]/4: 524.0 | |
| Seq161 | BRU-3021 | DO3A10CM-Dnal2-Arg-Trp-Ser-His-Darg-Trp-Bpa4-Pro-Az23m2po- | F, 5.36 | 1930 | Pos. ion: | 0.50 |
| | | NH₂ | | | [M + TFA + Na + H]/2: | |
| | | | | | 1043.8; | |
| | | | | | [M + TFA + 2H]/2: | |
| | | | | | 1021.8; | |
| | | | | | [M + TFA + 3H]/3: 681.6 | |
| Seq146 | BRU-2994 | DO3A10CM-Dnal2-Arg-Trp-Ser-His-Darg-Trp-Bpa4-Flp4-azaGly-NH₂ | D 7.57 | 1934 | Pos. ion: [M + H]: | 0.60 |
| | | | | | 1934.8, [M + Na + H]/2: | |
| | | | | | 978.8; [M + 2H]/2: 967.8; | |
| | | | | | [M + Na + 2H]/3: 652.8; | |
| | | | | | [M + 3H]/3: 645.8 | |
| Seq115 | BRU-2953 | DO3A10CM-Dnal2-Arg-Trp-Ser-His-Darg-Trp-Bpa4-Ppt4-azaGly-NH₂ | D, 8.92 | 1992 | Pos. ion: [M + H]: | 0.83 |
| | | | | | 1993.0; [M + H + Na]/2: | |
| | | | | | 1007.8; [M + 2H]/2: | |
| | | | | | 996.8; [M + 3H]/3: 665.0 | |

### HPLC Systems Used for Analysis of Peptides and N-terminus Derivatized Compounds.

System A. Column: Waters XTerra MS-C18, 4.6×50 mm; Particle size: 5 microns; Eluents: A: Water (0.1% TFA), B: Acetonitrile (0.1% TFA); Elution:
Initial condition 10% B, Linear gradient 10-25% B in 15 min; Flow rate: 3 mL/min; Detection: UV @ 220 nm.
System B. Column: Waters, XTerra MS-C18, 4.6×50 mm; Particle size: 5 microns; Eluents: A: Water (0.1% TFA), B: Acetonitrile (0.1% TFA); Elution: Initial condition: 10% B, linear gradient 10-40% B over 10 min; Flow rate: 3 mL/min; Detection: UV @ 230 and 254 nm.
System D. Column: Waters XTerra MS-C18, 4.6×50 mm; Particle size: 5 microns; Eluents: A: Water (0.1% TFA), B: acetonitrile (0.1% TFA); Elution: Initial condition: 10% B, linear gradient 10-40% B over 10 min; Flow rate: 3 mL/min; Detection: UV @ 220 and 230 nm.
System F. Column: Waters XTerra MS-C18, 4.6×50 mm; Particle size: 5 microns; Eluents: A: Water (0.1% TFA), B: Acetonitrile (0.1% TFA); Elution: Initial condition: 20% B, linear gradient 20-60% B over 10 min; Flow rate: 3 mL/min; Detection: UV @ 220 and 230 nm.

LHRH Peptides with Chelator on the C-terminus

| | | | | | | |
|---|---|---|---|---|---|---|
| Seq. | | | | | Retention | |
| ID | | | Synthetic | | Time (min)/ | |
| (C | BRU | | Methods | | HPLC | EC₅₀ |
| term) | Number | Sequence | Used | Mass | Method | μM |

| | | | | | | |
|---|---|---|---|---|---|---|
| 18 | 3103 | Sar-Arg-Trp-Ser-His-Darg-Trp-Bpa4-Pro-Dae- | A, B, C, D, H | 1775 | 2.83, (i) | 0.17 ± 0.07 |
| | | DO3A10CM | | | | |
| 6 | 3042 | Dnal2-Arg-Trp-Ser-His-Darg-Trp-Bpa4-Pro-Dabt14- | A, B, C, D, H | 1929 | 3.76, (i) | 0.20 ± 0.0 |
| | | DO3A10CM; | | | | |
| 17 | 3102 | Sar-Arg-Trp-Ser-His-Darg-Trp-Bpa4-Pro-Ampip2- | A, B, C, G, H | 1829 | 3.12, (i) | 0.20 ± 0.07 |
| | | DO3A10CM | | | | |
| 2 | 2991 | Dnal2-Arg-Trp-Ser-His-Darg-Trp-Bpa4-Pro-Da15o3pt- | A, B, C, D, H | 1945 | 3.64, (i) | 0.21 ± 0.06 |
| | | DO3A10CM | | | | |
| 9 | 3045 | Sar-Arg-Trp-Ser-His-Darg-Trp-Bpa4-Pro-Maz4dahp17- | A, B, C, D, H | 1860 | 5.09, (ii) | 0.21 ± 0.08 |
| | | DO3A10CM | | | | |
| 14 | 3080 | Sar-Arg-Trp-Ser-His-Darg-Trp-Bpa4-Pro-Bampy26- | A, B, C, D, H | 1852 | 5.57, (ii) | 0.23 ± 0.1 |
| | | DO3A10CM | | | | |
| 8 | 3044 | Sar-Arg-Trp-Ser-His-Darg-Trp-Bpa4-Pro-Bap14p- | A, B, C, D, H | 1915 | 5.15, (ii) | 0.24 ± 0.08 |
| | | DO3A10CM; | | | | |
| 4 | 3039 | Sar-Arg-Trp-Ser-His-Darg-Trp-Bpa4-Pro-Da15o3pt- | A, B, C, D, H | 1819 | 2.76, (i) | 0.28 ± 0.03 |
| | | DO3A10CM | | | | |
| 7 | 3043 | Sar-Arg-Trp-Ser-His-Darg-Trp-Bpa4-Pro-Da18o36oc- | A, B, C, E, H | 1863 | 6.18, (ii) | 0.33 ± 0.18 |
| | | DO3A10CM | | | | |
| 19 | 3117 | Sar-Arg-Trp-Ser-His-Darg-Trp-Bpa4-Am2prd- | A, B, C, G, H | 1718 | 2.89, (i) | 0.34 ± 0.0 |
| | | DO3A10CM | | | | |
| 5 | 3041 | Sar-Arg-Trp-Ser-His-Darg-Trp-Bpa4-Pro-Dabt14- | A, B, C, D, H | 1803 | 2.89, (i) | 0.36 ± 0.02 |
| | | DO3A10CM | | | | |
| 15 | 3085 | Sar-Arg-Trp-Ser-His-Darg-Trp-Bpa4-Pro-Dapt15- | A, B, C, D, H | 1817 | 3.25, (iii) | 0.40 ± 0.25 |
| | | DO3A10CM | | | | |
| 3 | 2992 | Ac-Dnal2-Arg-Trp-Ser-His-Darg-Trp-Bpa4-Pro- | A, B, C, D, H | 1987 | 4.31, (i) | 0.47 ± 0.02 |
| | | Da15o3pt-DO3A10CM | | | | |

### HPLC Systems Used for Analysis of Peptides and C-terminus Derivatized Compounds.

Column: X-Terra MS C₁₈ (Waters Corp.), RP; Particle size: 5.0μ; Solvent A: Water with 0.1% TFA (v/v) and Solvent B: Acetonitrile with 0.1% TFA (v/v); Elution rate; 3.0 mL/min; Detection at 220 nm.
Method (i): Initial conditions: 20% B; Gradient 20-60% B over 10 min
Method (ii): Initial conditions: 15% B; Gradient: 15-45% B over 15 min
Method (iii): Initial conditions: 20% B; Gradient: 20-60% B over 15 min

### REFERENCES

1. Schally, A. V.; Arimura, A.; Kastin, A. J.; Matsuo, H.; Baba, Y.; Redding, T. W.; Nair, R. M. G.; Debeljuk, L.; White, W. F. Gonadotropin-Releasing Hormone: One Polypeptide Regulates Secretion of Luteinizing and Follicle-Stimulating Hormones. Science 1971, 173, 1036-1038.
2. Schally, A. V.; Baba, Y.; Matsuo, H. Structure of the porcine LH- and FSH-releasing hormone. II. Confirmation of the proposed structure by conventional sequential analyses. Biochem. Biophys. Res. Commun. 1971, 44, 459-463.
3. Okon, E.; Koch, Y. Localization of gonadotropin-releasing hormone in the circumventricular organ of human brain. Nature (Lond.), 1977, 268, 445-447.
4. Seeburg, P. H.; Adelman, J. P. Characterization of cDNA for precursor of human luteinizing hormone releasing hormone. Nature (Lond.), 1984, 311, 666-668.
5. Oikawa, M.; Dargan, C.; Ny, T.; Hsuch, A. J. Expression of gonadotropin-releasing hormone and prothymosin-α messenger ribonucleic acid in the ovary. Endocrinology, 1990, 127, 2350-2356.
6. Palmon, A.; Ben-Aroya, N.; Tel-Or, S.; Burstein, Y.; Fridkin, M.; Koch, Y. The gene for the neuropeptide gonadotropin-releasing hormone is expressed in the mammary gland of the lactating rats, Proc. Natl. Acad. Sci., USA, 1994, 91, 5748-5751
7. Azad, N.; Emanuele, N. V.; Halloroan, M. M.; Tentler, J.; Kelley, M. R. Presence of luteinizing hormone-releasing hormone (LHRH) mRNA in rat spleen lymphocytes, Endocrinology, 1991, 128, 1679-1681.
8. Huirne, J. A; Lambalk, C. B. Gonadotropin-releasing-hormone-receptor antagonists. Lancet, 2001, 358, 1793-1803.
9. Schally, A. V. Hypothalamic hormones: From neuroendocrinology to cancer therapy. Anti-Cancer Drugs 1994, 5(2), 115-130.
10. Schally, A. V.; Comaru-Schally, A. M.; Nagy, A.; Kovacs, M.; Szepeshazi, K.; Plonowski, A.; Varga, J. L.; Halmos, G. Hypothalamic hormones and cancer. Front. Neuroendocrinology 2001, 22(4), 248-291.
11. Belchetz, P. E.; Plant, T. M.; Nakai, Y.; Keogh, E. J.; Knobil, E. Hypophysial responses to continuous and intermittent delivery of hypothalamic gonadotropin-releasing hormone. Science 1978, 202, 631-633.
12. Karten, M. J.; Rivier, J. E. Gonadotropin-releasing hormone analog design. Structure-function studies toward the development of agonists and antagonists: rationale and perspective. Endocr. Rev. 1986, 7, 44-66.
13. Dutta, A. S. Luteinizing Hormone-Releasing Hormone (LHRH) agonists. Drugs Future. 1988, 13, 43-57.
14. Filicori, M.; Flamigni, C. GnRH agonists and antagonists current clinical status. Drugs 1988, 35, 63-82.
15. Limonta, P.; Moretti, R. M.; Marelli, M. M.; Motta, M. The biology of gonadotropin hormone-releasing hormone: Role in the control of tumor growth and progression in humans. Front. Neuroendocrinol. 2003, 24, 279-295.
16. King, J. A.; Millar, R. P. Heterogeneity of vertebrate luteinizing hormone-releasing hormone. Science 1979, 206, 67-69.
17. Matsuo, H.; Baba, Y.; Nair, R. M. G.; Arimura, A.; Schally, A. V. Structure of the porcine LH- and FSH-releasing hormone. I. The proposed amino acid sequence. Biochem. Biophys. Res. Commun. 1971, 43, 1334-1339.
18. Miyamoto, K.; Hasegawa, Y.; Nomura, M.; Igarashi, M.; Kangawa, K.; Matsuo, H. Identification of the second gonadotropin-releasing hormone in chicken hypothalamus: evidence that gonadotropin secretion is probably controlled by two distinct gonadotropin-releasing hormones in avian species. Proc. Natl. Acad. Sci. USA 1984, 81, 3874-387.
19. Miyamoto, K.; Hasegawa, Y.; Igarashi, M.; Chino, N.; Sakakibara, S.; Kangawa, K.; Matsuo, H. Evidence that chicken hypothalamic luteinizing hormone-releasing hormone is [Gln⁸]-LHRH. Life Sci. 1983, 32, 1341-1347.
20. Ngamvongchon, S.; Lovejoy, D. A.; Fischer, W. H. Craig, A. G.; Nahorniak, C. S.; Peter, R. E.; Rivier, J. E.; Sherwood, N. M. Primary structures of two forms of gonadotropin-releasing hormone, one distinct and one conserved, from catfish brain. Mol. Cell. Neurosci. 1992, 3, 17-22.
21. Sherwood, N.; Eiden, L.; Brownstein, M.; Spiess, J.; Rivier, J.; Vale. W. Characterization of a teleost gonadotropin-releasing hormone. Proc. Natl. Acad. Sci. USA 1983, 80, 2794-2798.
22. Lovejoy, D. A.; Fischer, W. H.; Ngamvongchon, S.; Craig, A. G.; Nahorniak, C. S.; Peter, R. E.; Rivier, J. E.; Sherwood, N. M. Distinct sequence of gonadotropin-releasing hormone (GnRH) in dogfish brain provides insight into GnRH evolution. Proc. Natl. Acad. Sci. USA 1992, 89, 6373-6377.
23. Sherwood, N. M.; Sower, S. A.; Marshak, D. R.; Fraser, B. A.; Brownstein, M. J. Primary structure of gonadotropin-releasing hormone from lamprey brain. J. Biol. Chem. 1986, 261, 4812-4819.
24. King, J. A.; Steneveld, A. A.; Curlewis, J. D.; Rissman, E. F.; Millar, R. P. Identification of chicken GnRH II in brains of metatherian and early-evolved eutherian species of mammals. Regul. Pept. 1994, 54, 467-477.
25. Delloovade, T. L.; King, J. A.; Millar, R. P.; Rissman, E. F. Presence and differential distribution of distinct forms of immunoreactive Gonadotropin-Releasing Hormone in the musk shrew brain. Neuroendocrinol. 1993, 58, 166-177.
26. Kasten, T. L.; White, S. A.; Norton, T. T.; Bond, C. T.; Adelman, J. P.; Fernald, R. D. Characterization of two new preproGnRH mRNAs in the tree shrew: First direct evidence for mesencephalic GnRH gene expression in a placental mammal. Gen. Comp. Endocrinol. 1996, 104, 7-19.
27. Miyamoto, K.; Hasegawa, Y.; Igarashi, M.; Kangawa, K.; Matsuo, H. Structural determination of the second gonadotropin-releasing hormone (GnRH-II) in chicken hypothalamus. Peptide Chemistry 1984, 21, 99-104.
28. White, R. B.; Eisen, J. A.; Kaste, T. L.; Fernald. R. D. Second gene for gonadotropin-releasing hormone in humans. Proc. Natl. Acad. Sci. USA 1998, 95, 305-309.
29. Fernald, R. D.; White, R. B. Gonadotropin-releasing hormone genes; Phylogeny, structure and functions. Front. Neuroendocrinol., 1999, 20, 224-240.
30. Islami, D.; Chardonnens, D.; Campana, A.; Bischof, P. Comparison of the effects of GnRH-I and GnRH-II on HCG synthesis and secretion by first trimester trophoblast. Mol. Hum. Reprod. 2001, 7, 3-9.
31. Chou, C. S.; MacCalman, C. D.; Leung, P. C. Differential effects of Gonadotropin-Releasing Hormone I and II on the Urokinase-Type Plasminogen Activator/Plasminogen Activator inhibitor system in human decidual stromal cells in vitro. J. Clin. Endocrinol. Metab. 2003, 88, 3806-3815.
32. Chou, C. S.; Zhu, H.; MacCalman, C. D.; Leung, P. C. Regulatory Effects of Gonadotropin-Releasing Hormone (GnRH) I and GnRH II on the levels of Matrix Metalloproteinase (MMP)-2, MMP-9, and Tissue Inhibitor of Metalloproteinases-1 in primary cultures of human extravillous cytotrophoblasts. J. Clin. Endocrinol. Metab. 2003, 88, 4781-4790.
33. Kang, S. K.; Tai, C.-J.; Nathwan, P. S.; Leung, P. C. Differential regulation of two forms of Gonadotropin-Releasing Hormone messenger ribonucleic acid in human granulosa-luteal cells. Endocrinology 2001, 142, 182-192.
34. Kang, W.-C.; Lee, H.-S.; Parhar, I. S.; Kang, I. S. Expression of the second isoform of gonadotropin-releasing hormone (GnRH-II) in human endometrium throughout the menstrual cycle. Mol. Hum. Reprod. 2001, 7, 447-452.
35. Miller, R.; Lowe, S.; Conklin, D.; Pawson, A.; Maudsley, S.; Troskie, B.; Ott, T.; Millar, M.; Lincoln, G.; Sellar, R.; Faurholm, B.; Scobie, G.; Kuestner, R.; Terasawa, E.; Katz, A. A novel mammalian receptor for the evolutionarily conserved type II GnRH, PNAS, 2001, 98, 9636-9641.
36. Neill, J. D.; Duck, L. W.; Sellers, J. C.; Musgrove, L. C. A gonadotropin-releasing hormone (GnRH) receptor specific for GnRH-II in primates. Biochemical and Biophysical Research Communication, 2001, 282, 1012-1018,
37. Gründker, C.; Günthert, A. R.; Miller, R. P.; Emons, G. Expression of gonadotropin-releasing hormone II (GnRH-II) receptor in human endometrial and ovarian cancer cells and effects of GnRH-II on tumor cell proliferation, Journal of Clinical Endocrinology and Metabolism. 2002, 87, 1427-1430.
38. Siler-Khodr, T. M.; Yu, F.-Q.; Wei, P.; Tao, S.-X.; Liu, Y.-X. Contraceptive action of a Gonadotropin-Releasing Hormone II analog in the Rhesus monkey. J. Clin. Endocrinol. Metab. 2004, 89, 4513-4520.
39. Eidne, K. A.; Flanagan, C. A.; Millar, R. P. Gonadotropin-releasing hormone binding sites in human breast carcinoma. Science 1985, 229, 989-991.
40. Letsch, M.; Schally, A. V.; Szepeshazi, K.; Halmos, G.; Nagy, A. Preclinical evaluation of targeted cytotoxic Luteinizing Hormone-Releasing Hormone analogue AN-152 in androgen-Sensitive and insensitive prostate cancers. Clin. Cancer. Res. 2003, 9, 4505-4513.
41. Janaky, T.; Juhasz, A.; Bajusz, S.; Csernus, V.; Srkalovic, G.; Bokser, L.; Milovanovic, S.; Redding, T. W.; Rekasi, Z.; Schally, A. V.; Nagy, A. Analogues of luteinizing hormone-releasing hormone containing cytotoxic groups. Proc. Natl. Acad. Sci. USA 1992, 89, 972-976.
42. Bajusz, S.; Janaky, T.; Csernus, V.; Bokser, L.; Fekete, M.; Srkalovic, G.; Redding, T. W.; Schally, A. V. Highly potent metallopeptide analogues of luteinizing hormone-releasing hormone. Proc. Natl. Acad. Sci. USA 1989, 86, 6313-6317.
43. Zuckermann, R. N.; Martin, E. J.; Spellmeyer, D. C.; Stauber, G. B.; Shoemaker, K. R.; Kerr, J. M.; Figliozzi, G. M.; Goff, D. A.; Siani, M. A.; Simon, R. J.; Banville, S. C.; Brown, E, G.; Wang, L.; Richter, L. S.; Moos, W. H. Discovery of nanomolar ligands for 7-Transmembrane G-Protein-Coupled receptors from a diverse N-(Substituted)glycine peptoid library. J. Med. Chem. 1994, 37, 2678-2685.
44. Dutta, A. S.; Furr, B. J. A.; Giles, M. B.; Valcaccia, B. Synthesis and biological activity of highly active alpha-aza analogs of luliberin. J. Med. Chem. 1978, 21, 1018-1024.
45. Siler-Khodr, T. M.; Khodr, G. S, Non-mammalian gonadotropin-releasing hormone (GnRH) analogs and uses thereof in tumor cell growth regulation and cancer therapy. PCT Int. Appl. 2001, 49, pp. WO 2001074377, A120011011.
46. Fujino, M.; Kobayashi, S.; Obayashi, M.; Shinagawa, S.; Fukuda, T.; Kitada, C.; Nakayaga, R.; Yamazaki, I White, W. F.; Rippel, R. H. Structure-activity relationships in the C-terminal part of luteinizing hormone releasing hormone (LH-RH). Biochem. Biophys. Res. Commun. 1972, 49, 863-869.
47. Bunin, B. A. The Combinatorial Index; Academic Press; San Diego, 1998, 213-236.
48. Anderson, M. O.; Yu. H.; Peneranda, C.; Maddux, B. A.; Goldfine, I. D.; Youngren J. F.; Guy, K. Parallel synthesis of diarylureas and their evaluation as inhibitors of Insulin-Like Growth Factor Receptor. J. Comb. Chem. 2006, 8, 784-790.
49. Ahn, A.-E.; Kim, S. W.; Ro, S. Solid phase synthesis of azapeptides using an automatic synthesizer. Mol. Diversity. 1999, 4, 23-24.
50. Folkers, K.; Bowers, C. Y.; Lutz, W. B.; Friebel, K. J.; Kubiak, T.; Schircks, B.; Rampold, G. Synthesis and bioassay of antagonists of the luteinizing hormone releasing hormone having the azaGly10 moiety. Z. Naturforsch. 1982, 37b, 1075-1081.
51. Dutta, A. S.; Morley, J. S. α-Aza-analogs of biologically-active peptides. Pept. Proc. Eur. Pept. Symp., 14^{th}. 1976, 517-522.
52. Folkers, K.; Bowers, C. Y.; Stepinski, J.; Plucinski, T.; Sakagami, M.; Kubiak, T. Analogs of the luteinizing hormone releasing hormone having the Azagly10 moiety with antiovulatory activity. Z. Naturforsch. 1984, 39b, 528-532.
53. Koch, Y.; Baram, T.; Chobsieng, P.; Fridkin, M. Enzymic degradation of luteinizing hormone-releasing hormone (LH-RH) by hypothalamic tissue. Biochem. Biophys. Res. Commun. 1974, 61, 95-103.
54. Sega-Abramson, T.; Kitroser, H.; Levy, J.; Schally, A. V.; Sharoni, Y. Direct effects of luteinizing hormone-releasing hormone agonists and antagonists on MCF-7 mammary cancer cells. Proc. Natl. Acad. Sci. USA 1992, 89, 2336-2339.
55. Janaky, T.; Juhasz, A.; Rekasi, Z.; Serfozo, P.; Pinski, J.; Bokser, L.; Srkalovic, G.; Milovanovic, S.; Milovanovic, S.; Redding, T. W.; Halmos, G.; Nagy, A.; Schally, A. V. Short-chain analogs of luteinizing hormone-releasing hormone containing cytotoxic moieties. Proc. Natl. Acad. Sci. USA 1992, 89, 10203-10207.
56. Monahan, M. W.; Amoss, M. S.; Anderson, H. A.; Vale, W. Synthetic analogs of the hypothalamic luteinizing hormone releasing factor with increased agonist or antagonist properties. Biochemistry 1973, 12, 4616-4620.
57. Coy, D. H.; Coy, E. J.; Schally, A. V.; Vilchez, -M. J.; Arimura, A. Synthesis and biological properties of [D-Ala-6, des-Gly-NH₂-10]-LH-RH ethylamide, a peptide with greatly enhanced LH- and FSH-releasing activity. Biochem. Biophys. Res. Commun. 1974, 57, 335-338.
58. Fujino, M.; Yamazaki, I.; Kobayashi, S.; Fukuda, T.; Shinagawa, S.; Nakayama, R.; White, W. F.; Rippel, R. H. Some analogs of luteinizing hormone releasing hormone (LH-RH) having intense ovulation-inducing activity. Biochem. Biophys. Res. Commun. 1974, 57, 1248-1254.
59. Haviv, F.; Fitzpatrick, T. D.; Nichols, C. J.; Swenson, R. E.; Bush, E. N.; Daiz, G.; Nguyen, A. T.; Nellans, H. N.; Hoffman, D, J.; Ghanbari, H.; Johnson, E. S.; Love, S.; Cybulski, V. A.; Greer, J. Stabilization of the N-terminal residues of luteinizing hormone-releasing hormone agonists and the effect on pharmacokinetics. J. Med. Chem. 1992, 35, 3890-3894.
60. Haviv, F.; Palabrica, C. A.; Bush, E. N.; Daiz, G.; Johnson, E. S.; Love, S.; Greer, J. Active reduced-size hexapeptide analogs of luteinizing hormone-releasing hormone. J. Med. Chem. 1989, 32, 2340-2344.
61. International Publication No. WO 2005/074645 A2.
62. Fu, H.; Xue, S.; Zhu, Q.; Tao, Z.; Zhang, J.; Day, A. I. Investigation of host-guest compounds of Cucurbit[n=5-8]uril with some ortho aminopyridines and bispyridine. Journal of Inclusion Phenomena and Macrocyclic Chemistry, 2005, 52, 101-107.
63. Krakowiak, K. E.; Bradshaw, J. S. Selective protection of the primary amine functions of linear tetramines using the trityl group. Synth. Commun., 1998, 28, 3451-3459.

We claim:

1. A peptide of the formula
X₁-X₂-X₃-X₄-X₅-X₆-X₂-X₈-X₉-X₁₀,
wherein:
X₁ is an optional component which, when present, is selected from the group consisting of Arg, His, pGlu, Sar, Dnal2, Ac-Amfe4, Ac-Dnal2, Dtpi, Damfe4, Bip, Dbpa4, Tpi, Mogly, Ampha4, Dnal1, Qua3, Thy, Atdc2, Dtyr, Apsp, Hpgly, Datdc2 and Ahgly;
X₂ is selected from the group consisting of Arg, His, Gufe4, Damfe4, Ampg4, Darg and Ampa4;
X₃ is selected from the group consisting of Trp, Arg, Phe, Nal2, Nal1 and Amfe4;
X₄ is selected from the group consisting of Ser, Met, Asn, Amfe4 and Dap;
X₅ is selected from the group consisting of His, Arg, Orn and Fur3ala;
X₆ is selected from the group consisting of Arg and Darg;
X₇ is Trp;
X₈ is selected from the group consisting of Bpa4, Tyr and Nal2;
X₉ is selected from the group consisting of Pro, Am2prd, Thz, Hypt4, Ampc4, Ampt4, Pip, Flp4 and Aze;
or X₈ and X₉ together can form a dipeptide isostere X₈-Ψ(CH₂N)—X₉; and
X₁₀ is an optional component which, when present, is selected from the group consisting of azaGly-NH₂, Gly-Arg-NH₂, Gly-Gln-NH₂, Da15o3t, Gua, Ap, Az34m3buo-NH₂, Pheo1, Mo2abn, A1gua5o3pt and Az23m2po-NH₂;
with the proviso that the peptide is not pGlu-His-Trp-Ser-His-Darg-Trp-Tyr-Pro-azaGly-NH₂ (SEQ ID NO: 199).

2. A peptide of the formula
X₁-X₂-X₃-X₄-X₅-X₆-X₇-X₈-X₉-linker-DL,
wherein X₁ through X₉ are as defined in claim 1 and DL is a component containing a label detectable via scintigraphic imaging, magnetic resonance imaging, positron emission tomography imaging, single photon emission computed tomography imaging, a hand-held probe, ultrasound contrast analysis or optical imaging, or an enzymatically cleavable label.

3. The peptide according to claim 2, wherein the linker is selected from the group consisting of Dae, Dabt14, Ampip2, Da15o3pt, Maz4dahp17, Bampy 26, Bap14p, Da18o36oc and Dapt15.

4. The peptide according to claim 3, wherein DL is a chelator selected from the group consisting of DO3A10CM, DTPA, NOTA, PnAO, oxa PnAO and N,N-dimethyl-Gly-Ser-Cys.

5. The peptide according to claim 4, wherein the chelator is DO3A10CM.

6. The peptide according to claim 4, wherein the chelator is complexed with a suitable metal radionuclide.

7. The peptide according to claim 6, wherein the radionuclide is selected from the group consisting of ¹⁷⁷Lu, ^{99m}Tc, ¹¹¹In, ⁶⁸Ga, ⁶⁴Cu, ⁹⁰Y, ¹⁸⁶Re, and ¹⁸⁸Re.

8. The peptide according to claim 4, wherein the chelator is not complexed with a metal.

9. A peptide of the formula
DL-optional linker-X₁-X₂-X₃-X₄-X₅-X₆-X₇-X₈-X₉-X₁₀,
wherein X₁ through X₁₀ are as defined in claim 1 and DL is a component containing a label detectable via scintigraphic imaging, magnetic resonance imaging, positron emission tomography imaging, single photon emission computed tomography imaging, a hand-held probe, ultrasound contrast analysis or optical imaging, or an enzymatically cleavable label.

10. The peptide according to claim 9, wherein the linker, when present, is selected from the group consisting of Da48oa, Amb4, Gly, Dap, Gly-Abz4, Lys and Dlys.

11. The peptide according to claim 10, wherein DL is a chelator selected from the group consisting of DO3A10CM, DTPA, NOTA, PnAO, oxa PnAO and N,N-dimethyl-Gly-Ser-Cys.

12. The peptide according to claim 11, wherein the chelator is DO3A10CM.

13. The peptide according to claim 11, wherein the chelator is complexed with a suitable metal radionuclide.

14. The peptide according to claim 13, wherein the radionuclide is selected from the group consisting of ¹⁷⁷Lu, ^{99m}Tc, ¹¹¹In, ⁶⁸Ga, ⁶⁴Cu, ⁹⁰Y, ¹⁸⁶Re, and ¹⁸⁸Re.

15. The peptide according to claim 11, wherein the chelator is not complexed with a metal.

16. A peptide selected from the group consisting of BRU-3103 (SEQ ID NO: 8), -3042 (SEQ ID NO: 9), -3102 (SEQ ID NO: 8), -2991 (SEQ ID NO: 8), -3045 (SEQ ID NO: 8), -3080 (SEQ ID NO: 8), -3044 (SEQ ID NO: 8), -3039 (SEQ ID NO: 8), -3043 (SEQ ID NO: 8), -3117 (SEQ ID NO: 10), -3041 (SEQ ID NO: 8), -3085 (SEQ ID NO: 8), -2992 (SEQ ID NO: 11), -2441 (SEQ ID NO: 12), -2734 (SEQ ID NO: 13), -3007 (SEQ ID NO: 8), -2439 (SEQ ID NO: 14), -2839 (SEQ ID NO: 15), -2803 (SEQ ID NO: 16), -2821 (SEQ ID NO: 17), -2822 (SEQ ID NO: 18), -3100 (SEQ ID NO: 19), -3115 (SEQ ID NO: 20), -3072 (SEQ ID NO: 21), -2964 (SEQ ID NO: 22), -3105 (SEQ ID NO:23), -2968 (SEQ ID NO: 24), -2969 (SEQ ID NO: 25), -3068 (SEQ ID NO: 26), -2959 (SEQ ID NO: 27), -3104 (SEQ ID NO: 28), -3111 (SEQ ID NO: 29), -2757 (SEQ ID NO: 30), -3058 (SEQ ID NO: 31), -2956 (SEQ ID NO: 32), -2952 (SEQ ID NO: 33), -2963 (SEQ ID NO: 34), -3070 (SEQ ID NO: 35), -3095 (SEQ ID NO: 36), -3081 (SEQ ID NO: 37), -3031 (SEQ ID NO: 38), -3050 (SEQ ID NO: 39), -3071 (SEQ ID NO: 40), -3053 (SEQ ID NO: 41), -3062 (SEQ ID NO: 42), -2813 (SEQ ID NO: 43), -2997 (SEQ ID NO: 44), -2796 (SEQ ID NO: 45), -3060 (SEQ ID NO: 46), -2961 (SEQ ID NO: 47), -2996 (SEQ ID NO: 48), -3094 (SEQ ID NO: 49), -2811 (SEQ ID NO: 50), -2869 (SEQ ID NO: 51), -3049 (SEQ ID NO: 52), -3027 (SEQ ID NO: 53), -3096 (SEQ ID NO: 54), -2993 (SEQ ID NO: 55), -3057 (SEQ ID NO: 56), -3069 (SEQ ID NO: 57), -3107 (SEQ ID NO: 58), -3055 (SEQ ID NO: 59), -2960 (SEQ ID NO: 60), -2984 (SEQ ID NO: 24), -2955 (SEQ ID NO: 61), -2995 (SEQ ID NO: 62), -3059 (SEQ ID NO: 63), -3098 (SEQ ID NO: 64), -3006 (SEQ ID NO: 24), -3054 (SEQ ID NO: 65), -3106 (SEQ ID NO: 66), -2696 (SEQ ID NO: 67), -2967 (SEQ ID NO: 68), -3056 (SEQ ID NO: 69), -3099 (SEQ ID NO: 70), -2797 (SEQ ID NO: 71), -2983 (SEQ ID NO: 24), -3020 (SEQ ID NO: 24), -3097 (SEQ ID NO: 72), -2985 (SEQ ID NO: 24), -2666 (SEQ ID NO: 73), -2962 (SEQ ID NO: 74), -3025 (SEQ ID NO: 75), -3063 (SEQ ID NO: 76), -2971 (SEQ ID NO: 77), -2876 (SEQ ID NO: 78), -3002 (SEQ ID NO: 79), -3021 (SEQ ID NO: 24), -2994 (SEQ ID NO: 80), and -2953 (SEQ ID NO: 81).

17. A peptide of the formula
DL₁-optional linker-X₁-X₂-X₃-X₄-X₅-X₆-X₂-X₈-X₉-X₁₀-linker-DL₂,
wherein X₁ through X₁₀ are as defined in claim 1; one of DL₁ and DL₂ is a chelator optionally complexed with a metal radionuclide; and the other is an optical imaging agent.

18. A peptide of the formula
X₁-X₂-X₃-X₄-X₅-X₆-X₇-X₈-X₉-X₁₀,
wherein X₁ through X₁₀ are as defined in claim 1; and wherein one of X₁ through X₁₀, or an additional residue X₁₁ bound either to X₁ or X₁₀, is labeled with a radioisotope selected from the group consisting of ¹²³I, ¹²⁴I, ¹²⁵I and ¹³¹I.

19. An LHRH-analog peptide of the formula
X₁-X₂-X₃-Ser-X₅-Darg-X₇-X₈-Pro-azaGlyNH₂,
wherein:
X₁ is selected from the group consisting of Arg, His, pGlu, Sar, Dnal2, Ac-Amfe4, Ac-Dnal2, Dtpi, Damfe4, Bip, Dbpa4, Tpi, Mogly, Ampha4, Dnal1, Qua3, Thy, Atdc2, Dtyr, Apsp, Hpgly, Datdc2 and Ahgly;
X₂ is selected from the group consisting of Arg, His, Gufe4, Damfe4, Ampg4, Darg and Ampa4;
X₃ is selected from the group consisting of Trp and Tyr;
X₅ is selected from the group consisting of His, Leu and Tyr;
X₂ is selected from the group consisting of Leu and Trp; and
X₈ is selected from the group consisting of Bpa4 and Nal2, or the Bpa4 or Nal2 at position 8 can form a dipeptide Ψ(CH₂N) isostere with the Pro at position 9.

20. The peptide according to claim 19,
wherein X₁ is pGlu and X₂ is His.

21. An analog peptide according to claim 19 which further is conjugated at the N- and/or C-terminus to a component containing a label detectable via scintigraphic imaging, magnetic resonance imaging, positron emission tomography imaging, single photon emission computed tomography imaging, a hand-held probe, ultrasound contrast analysis or optical imaging, or an enzymatically cleavable label.

22. A metabolically stabilized LHRH-II analog of the formula
X₁-X₂-Trp-Ser-His-X₆-Trp-X₈-X₉-GlyNH₂,
wherein X₁ is selected from the group consisting of pGlu, Dnal2 and Sar; X₂ is Arg; X₆ is Darg; X₈ is Bpa4; and X₉ is selected from the group consisting of Pro, Am2prd, Thz, Hypt4, Ampc4, Ampt4, Pip, Flp4 and Aze; and
wherein when X₉ is Pro, it and the Bpa4 at position 8 together form a dipeptide Ψ(CH₂N) isostere.

23. An analog according to claim 22 which further is conjugated at the N- and/or C-terminus to a component containing a label detectable via scintigraphic imaging, magnetic resonance imaging, positron emission tomography imaging, single photon emission computed tomography imaging, a hand-held probe, ultrasound contrast analysis or optical imaging, or an enzymatically cleavable label.

24. A pharmaceutical composition comprising a therapeutically effective amount of a peptide according to any one of claims 1-23 and a pharmaceutically acceptable carrier.

25. A method for targeted therapy of prostate, ovarian or breast cancer, which comprises administering to a patient in need of such therapy a therapeutically effective amount of a peptide according to claim 1.

26. A method for targeted radiotherapy of prostate, ovarian or breast cancer, which comprises administering to a patient in need of such therapy a therapeutically effective amount of a peptide-chelator conjugate according to any one of claims 2, 9 and 17.

27. The method according to claim 26, wherein the peptide is conjugated to a chelator complexed with a radionuclide selected from the group consisting of ¹⁷⁷Lu, ⁹⁰Y, ⁶⁴Cu, ¹⁰⁵Rh, ¹¹¹In, ₁₁₇ₘSn, ¹⁴⁹Pm, ¹⁵³Sm, ¹⁶¹Tb, ¹⁶⁶Dy, ¹⁶⁶Ho, ¹⁷⁵Yb, ^{186/188}Re and ¹⁹⁹Au.

28. A method for targeted radiotherapy of prostate, ovarian or breast cancer, which comprises administering to a patient in need of such therapy a therapeutically effective amount of a peptide according to claim 18 labeled with ¹²⁵I or ¹³¹I.

29. A method for targeted imaging in a patient, which comprises administering to the patient a suitable amount of a peptide-detectable-label conjugate according to any one of claims 2, 9 and 17 and using the appropriate imaging technology to locate and quantitate the bound label.

30. The method according to claim 29 for localizing tumors in, and/or evaluating the potential for treatment of, a patient with prostate, ovarian or breast cancer.

31. The method according to claim 29, wherein the peptide is conjugated to a chelator complexed with a radionuclide selected from the group consisting of ^{99m}Tc, ¹¹¹In, ⁶⁴Cu, ⁶⁷Ga and ⁶⁸Ga.

32. A method for targeted imaging in a patient, which comprises administering to the patient a suitable amount of a peptide according to claim 18 labeled with ¹²³I, ¹²⁴I or ¹³¹I and using the appropriate scintigraphy technology to locate and quantitate the bound label.

33. The method according to claim 32 for localizing tumors in, and/or evaluating the potential for treatment of, a patient with prostate, ovarian or breast cancer.


SEQUENCE LISTING (199 sequences)

SEQ ID NO: 1 - protein, 10 aa, Homo sapiens
  MISC_FEATURE (1)..(1): L-pyroglutamic acid
XHWSYGLRPX

SEQ ID NO: 2 - protein, 10 aa, Gallus gallus
  MISC_FEATURE (1)..(1): L-pyroglutamic acid
XHWSHGWYPX

SEQ ID NO: 3 - protein, 10 aa, Gallus gallus
  MISC_FEATURE (1)..(1): L-pyroglutamic acid
XHWSYGLQPX

SEQ ID NO: 4 - protein, 10 aa, Unknown
  Catfish--unknown genus and species
XHWSHGLNPX

SEQ ID NO: 5 - protein, 10 aa, Salmo sp.
  MISC_FEATURE (1)..(1): L-pyroglutamic acid
XHWSYGWLPX

SEQ ID NO: 6 - protein, 10 aa, Unknown
  Dogfish--unknown genus and species
XHWSHGWLPX

SEQ ID NO: 7 - protein, 10 aa, unknown
  lamprey--unknown genus and species
XHYSLEWKPX

SEQ ID NO: 8 - protein, 9 aa, Artificial Sequence
  synthetic peptide
XRWSHXWXP

SEQ ID NO: 9 - protein, 9 aa, Artificial Sequence
  synthetic peptide
XRWSHXWXP

SEQ ID NO: 10 - protein, 8 aa, Artificial Sequence
  synthetic peptide
XRWSHXWX

SEQ ID NO: 11 - protein, 9 aa, Artificial Sequence
  synthetic peptide
XRWSHXWXP

SEQ ID NO: 12 - protein, 10 aa, Artificial Sequence
  synthetic peptide
XHWSHXWXPX

SEQ ID NO: 13 - protein, 10 aa, Artificial Sequence
  synthetic peptide
XHWSHXWXPX

SEQ ID NO: 14 - protein, 10 aa, Artificial Sequence
  synthetic peptide
XHWSHXWYPX

SEQ ID NO: 15 - protein, 10 aa, Artificial Sequence
  synthetic peptide
XHWSHXWXPX

SEQ ID NO: 16 - protein, 10 aa, Artificial Sequence
  synthetic peptide
XHWSHXWXPX

SEQ ID NO: 17 - protein, 10 aa, Artificial Sequence
  synthetic peptide
XHWSHXWXPX

SEQ ID NO: 18 - protein, 10 aa, Artificial Sequence
  synthetic peptide
XHWSHXWXPX

SEQ ID NO: 19 - protein, 9 aa, Artificial Sequence
  synthetic peptide
XRWSHXWXP

SEQ ID NO: 20 - protein, 10 aa, Artificial Sequence
  synthetic peptide
XRWXHXWXPX

SEQ ID NO: 21 - protein, 10 aa, Artificial Sequence
  synthetic peptide
XRWSHXWXXX

SEQ ID NO: 22 - protein, 10 aa, Artificial Sequence
  synthetic peptide
XRWMHXWXPX

SEQ ID NO: 23 - protein, 10 aa, Artificial Sequence
  synthetic peptide
XRXSHXWXPX

SEQ ID NO: 24 - protein, 9 aa, Artificial Sequence
  synthetic peptide
XRWSHXWXP

SEQ ID NO: 25 - protein, 11 aa, Artificial Sequence
  synthetic peptide
XRWSHXWXPGX

SEQ ID NO: 26 - protein, 11 aa, Artificial Sequence
  synthetic peptide
GXRWSHXWXPX

SEQ ID NO: 27 - protein, 10 aa, Artificial Sequence
  synthetic peptide
XRWSRXWXPX

SEQ ID NO: 28 - protein, 10 aa, Artificial Sequence
  synthetic peptide
XRXSHXWXPX

SEQ ID NO: 29 - protein, 10 aa, Artificial Sequence
  synthetic peptide
XRRSHXWXPX

SEQ ID NO: 30 - protein, 10 aa, Artificial Sequence
  synthetic peptide
XHWSHXWXPX

SEQ ID NO: 31 - protein, 10 aa, Artificial Sequence
  synthetic peptide
XXWSHXWXPX

SEQ ID NO: 32 - protein, 10 aa, Artificial Sequence
  synthetic peptide
XRWNHXWXPX

SEQ ID NO: 33 - protein, 10 aa, Artificial Sequence
  synthetic peptide
XRWSHXWXXX

SEQ ID NO: 34 - protein, 11 aa, Artificial Sequence
  synthetic peptide
XXRWSHXWXPX

SEQ ID NO: 35 - protein, 10 aa, Artificial Sequence
  synthetic peptide
XRWSHXWXPX

SEQ ID NO: 36 - protein, 10 aa, Artificial Sequence
  synthetic peptide
XXWSHXWXPX

SEQ ID NO: 37 - protein, 10 aa, Artificial Sequence
  synthetic peptide
XRWSHXWXPX

SEQ ID NO: 38 - protein, 10 aa, Artificial Sequence
  synthetic peptide
XRWSHRWXPX

SEQ ID NO: 39 - protein, 10 aa, Artificial Sequence
  synthetic peptide
RRWSHXWXPX

SEQ ID NO: 40 - protein, 10 aa, Artificial Sequence
  synthetic peptide
XRWSHXWXPX

SEQ ID NO: 41 - protein, 11 aa, Artificial Sequence
  synthetic peptide
GXRWSHXWXPX

SEQ ID NO: 42 - protein, 10 aa, Artificial Sequence
  synthetic peptide
XRWSHXWXXX

SEQ ID NO: 43 - protein, 10 aa, Artificial Sequence
  synthetic peptide
XRWSHXWXPX

SEQ ID NO: 44 - protein, 10 aa, Artificial Sequence
  synthetic peptide
XRWSHXWXPX

SEQ ID NO: 45 - protein, 11 aa, Artificial Sequence
  synthetic peptide
GXHWSHXWXPX

SEQ ID NO: 46 - protein, 10 aa, Artificial Sequence
  synthetic peptide
XXWSHXWXPX

SEQ ID NO: 47 - protein, 10 aa, Artificial Sequence
  synthetic peptide
XRWSXXWXPX

SEQ ID NO: 48 - protein, 10 aa, Artificial Sequence
  synthetic peptide
XRWSHXWXXX

SEQ ID NO: 49 - protein, 10 aa, Artificial Sequence
  synthetic peptide
XRWSHXWXPX

SEQ ID NO: 50 - protein, 9 aa, Artificial Sequence
  synthetic peptide
HWSHXWXPX

SEQ ID NO: 51 - protein, 10 aa, Artificial Sequence
  synthetic peptide
XHWSHXWXPX

SEQ ID NO: 52 - protein, 10 aa, Artificial Sequence
  synthetic peptide
XXWSHXWXPX

SEQ ID NO: 53 - protein, 10 aa, Artificial Sequence
  synthetic peptide
XRWSHXWXPX

SEQ ID NO: 54 - protein, 11 aa, Artificial Sequence
  synthetic peptide
XXRWSHXWXPX

SEQ ID NO: 55 - protein, 10 aa, Artificial Sequence
  synthetic peptide
XRWSHXWXXX

SEQ ID NO: 56 - protein, 10 aa, Artificial Sequence
  synthetic peptide
XRWSHXWXPX

SEQ ID NO: 57 - protein, 10 aa, Artificial Sequence
  synthetic peptide
XRWSHXWXPX

SEQ ID NO: 58 - protein, 10 aa, Artificial Sequence
  synthetic peptide
XRXSHXWXPX

SEQ ID NO: 59 - protein, 10 aa, Artificial Sequence
  synthetic peptide
XRWSHXWXPX

SEQ ID NO: 60 - protein, 10 aa, Artificial Sequence
  synthetic peptide
XRWSXXWXPX

SEQ ID NO: 61 - protein, 10 aa, Artificial Sequence
  synthetic peptide
XRWXHXWXPX

SEQ ID NO: 62 - protein, 10 aa, Artificial Sequence
  synthetic peptide
XRWSHXWXXX

SEQ ID NO: 63 - protein, 10 aa, Artificial Sequence
  synthetic peptide
XXWSHXWXPX

SEQ ID NO: 64 - protein, 11 aa, Artificial Sequence
  synthetic peptide
KXRWSHXWXPX

SEQ ID NO: 65 - protein, 10 aa, Artificial Sequence
  synthetic peptide
XRWSHXWXPX

SEQ ID NO: 66 - protein, 10 aa, Artificial Sequence
  synthetic peptide
XRFSHXWXPX

SEQ ID NO: 67 - protein, 11 aa, Artificial Sequence
  synthetic peptide
XXHWSHXWXPX

SEQ ID NO: 68 - protein, 9 aa, Artificial Sequence
  synthetic peptide
XRWSHXWXW

SEQ ID NO: 69 - protein, 10 aa, Artificial Sequence
  synthetic peptide
XRWSHXWXPX

SEQ ID NO: 70 - protein, 11 aa, Artificial Sequence
  synthetic peptide
XXRWSHXWXPX

SEQ ID NO: 71 - protein, 11 aa, Artificial Sequence
  synthetic peptide
GXHWSHXWXPX

SEQ ID NO: 72 - protein, 11 aa, Artificial Sequence
  synthetic peptide
KXRWSHXWXPX

SEQ ID NO: 73 - protein, 10 aa, Artificial Sequence
  synthetic peptide
XHWSHXWXPX

SEQ ID NO: 74 - protein, 11 aa, Artificial Sequence
  synthetic peptide
GXRWSHXWXPX

SEQ ID NO: 75 - protein, 10 aa, Artificial Sequence
  synthetic peptide
HRWSHXWXPX

SEQ ID NO: 76 - protein, 10 aa, Artificial Sequence
  synthetic peptide
XRWSHXWXPX

SEQ ID NO: 77 - protein, 11 aa, Artificial Sequence
  synthetic peptide
XRWSHXWXPGX

SEQ ID NO: 78 - protein, 11 aa, Artificial Sequence
  synthetic peptide
GXHWSHXWXPX

SEQ ID NO: 79 - protein, 11 aa, Artificial Sequence
  synthetic peptide
XXRWSHXWXPX

SEQ ID NO: 80 - protein, 10 aa, Artificial Sequence
  synthetic peptide
XRWSHXWXXX

SEQ ID NO: 81 - protein, 10 aa, Artificial Sequence
  synthetic peptide
XRWSHXWXXX

SEQ ID NO: 82 - protein, 10 aa, Artificial Sequence
  synthetic peptide
XHWSHXWYPX

SEQ ID NO: 83 - protein, 10 aa, Artificial Sequence
  synthetic peptide
XHWSHXWYPX

SEQ ID NO: 84 - protein, 10 aa, Artificial Sequence
  synthetic peptide
XRWSHXWXPG

SEQ ID NO: 85 - protein, 11 aa, Artificial Sequence
  synthetic peptide
XRWSHXWXPGX

SEQ ID NO: 86 - protein, 7 aa, Artificial Sequence
  synthetic peptide
XRWSHXW

SEQ ID NO: 87 - protein, 8 aa, Artificial Sequence
  synthetic peptide
XRWSHXWX

SEQ ID NO: 88 - protein, 10 aa, Artificial Sequence
  synthetic peptide
XHWSHXWYPX

SEQ ID NO: 89 - protein, 10 aa, Artificial Sequence
  synthetic peptide
XHWSHXWYPX

SEQ ID NO: 90 - protein, 10 aa, Artificial Sequence
  synthetic peptide
XHWSHXWXPX

SEQ ID NO: 91 - protein, 10 aa, Artificial Sequence
  synthetic peptide
XHWSHXWXPX

SEQ ID NO: 92 - protein, 10 aa, Artificial Sequence
  synthetic peptide
XHWSHXWXPX

SEQ ID NO: 93 - protein, 10 aa, Artificial Sequence
  synthetic peptide
XHWSHXWXPX

SEQ ID NO: 94 - protein, 10 aa, Artificial Sequence
  synthetic peptide
XHWSHXWXPX

SEQ ID NO: 95 - protein, 10 aa, Artificial Sequence
  synthetic peptide
XHWSHXWXPX

SEQ ID NO: 96 - protein, 10 aa, Artificial Sequence
  synthetic peptide
XHWSHXWXPX

SEQ ID NO: 97 - protein, 10 aa, Artificial Sequence
  synthetic peptide
XHWSHXWXPX

SEQ ID NO: 98 - protein, 10 aa, Artificial Sequence
  synthetic peptide
XHWSHXWRPX

SEQ ID NO: 99 - protein, 10 aa, Artificial Sequence
  synthetic peptide
XHWSHXWWPX

SEQ ID NO: 100 - protein, 10 aa, Artificial Sequence
  synthetic peptide
XHWSHXWXPX

SEQ ID NO: 101 - protein, 10 aa, Artificial Sequence
  synthetic peptide
XHWSHXWXPX

SEQ ID NO: 102 - protein, 10 aa, Artificial Sequence
  synthetic peptide
XHWSHXWFPX

SEQ ID NO: 103 - protein, 10 aa, Artificial Sequence
  synthetic peptide
XHWSHXWXPX

SEQ ID NO: 104 - protein, 10 aa, Artificial Sequence
  synthetic peptide
XHWSHXWYPX

SEQ ID NO: 105 - protein, 10 aa, Artificial Sequence
  synthetic peptide
XHWSHXWXPX

SEQ ID NO: 106 - protein, 10 aa, Artificial Sequence
  synthetic peptide
XHWSHXWHPX

SEQ ID NO: 107 - protein, 10 aa, Artificial Sequence
  synthetic peptide
XHWSHXWXPX

SEQ ID NO: 108 - protein, 10 aa, Artificial Sequence
  synthetic peptide
XHWSHXWXPX

SEQ ID NO: 109 - protein, 9 aa, Artificial Sequence
  synthetic peptide
XHWSHXWPX

SEQ ID NO: 110 - protein, 10 aa, Artificial Sequence
  synthetic peptide
XHWSHXWXPX

SEQ ID NO: 111 - protein, 10 aa, Artificial Sequence
  synthetic peptide
XHWSHXWXPX

SEQ ID NO: 112 - protein, 10 aa, Artificial Sequence
  synthetic peptide
XHWSHXWXPX

SEQ ID NO: 113 - protein, 10 aa, Artificial Sequence
  synthetic peptide
XHWSHXWXPX

SEQ ID NO: 114 - protein, 10 aa, Artificial Sequence
  synthetic peptide
GHWSHXWXPX

SEQ ID NO: 115 - protein, 10 aa, Artificial Sequence
  synthetic peptide
FHWSHXWXPX

SEQ ID NO: 116 - protein, 10 aa, Artificial Sequence
  synthetic peptide
XHWSHXWXPX

SEQ ID NO: 117 - protein, 10 aa, Artificial Sequence
  synthetic peptide
XHWSHXWXPX

SEQ ID NO: 118 - protein, 10 aa, Artificial Sequence
  synthetic peptide
XHWSHXWXPX

SEQ ID NO: 119 - protein, 10 aa, Artificial Sequence
  synthetic peptide
XHWSHXWXPX

SEQ ID NO: 120 - protein, 10 aa, Artificial Sequence
  synthetic peptide
XHWSHXWXPX

SEQ ID NO: 121 - protein, 10 aa, Artificial Sequence
  synthetic peptide
XHWSHXWXPX

SEQ ID NO: 122 - protein, 10 aa, Artificial Sequence
  synthetic peptide
XHWSHXWXPX

SEQ ID NO: 123 - protein, 10 aa, Artificial Sequence
  synthetic peptide
XHWSHXWXPX

SEQ ID NO: 124 - protein, 10 aa, Artificial Sequence
  synthetic peptide
XHWSHXWXPX

SEQ ID NO: 125 - protein, 10 aa, Artificial Sequence
  synthetic peptide
XRWSHXWXPX

SEQ ID NO: 126 - protein, 10 aa, Artificial Sequence
  synthetic peptide
XXWSHXWXPX

SEQ ID NO: 127 - protein, 10 aa, Artificial Sequence
  synthetic peptide
XFWSHXWXPX

SEQ ID NO: 128 - protein, 10 aa, Artificial Sequence
  synthetic peptide
XXWSHXWXPX

SEQ ID NO: 129 - protein, 10 aa, Artificial Sequence
  synthetic peptide
XXWSHXWXPX

SEQ ID NO: 130 - protein, 10 aa, Artificial Sequence
  synthetic peptide
XXWSHXWXPX

SEQ ID NO: 131 - protein, 10 aa, Artificial Sequence
  synthetic peptide
XXWSHXWXPX

SEQ ID NO: 132 - protein, 10 aa, Artificial Sequence
  synthetic peptide
XYWSHXWXPX

SEQ ID NO: 133 - protein, 10 aa, Artificial Sequence
  synthetic peptide
XXWSHXWXPX

SEQ ID NO: 134 - protein, 10 aa, Artificial Sequence
  synthetic peptide
XRWSHXWXPX

SEQ ID NO: 135 - protein, 10 aa, Artificial Sequence
  synthetic peptide
XXWSHXWXPX

SEQ ID NO: 136 - protein, 10 aa, Artificial Sequence
  synthetic peptide
XKWSHXWXPX

SEQ ID NO: 137 - protein, 11 aa, Artificial Sequence
  synthetic peptide
GPHWSHXWXPX

SEQ ID NO: 138 - protein, 11 aa, Artificial Sequence
  synthetic peptide
GXHWSHXWXPX

SEQ ID NO: 139 - protein, 11 aa, Artificial Sequence
  synthetic peptide
GXHWSHXWXPX

SEQ ID NO: 140 - protein, 11 aa, Artificial Sequence
  synthetic peptide
GXHWSHXWXPX

SEQ ID NO: 141 - protein, 11 aa, Artificial Sequence
  synthetic peptide
GXHWSHXWXPX

SEQ ID NO: 142 - protein, 11 aa, Artificial Sequence
  synthetic peptide
GGHWSHXWXPX

SEQ ID NO: 143 - protein, 10 aa, Artificial Sequence
  synthetic peptide
XRWSHXWXPX

SEQ ID NO: 144 - protein, 11 aa, Artificial Sequence
  synthetic peptide
GXRWSHXWXPX

SEQ ID NO: 145 - protein, 11 aa, Artificial Sequence
  synthetic peptide
GXRWSHXWXPX

SEQ ID NO: 146 - protein, 10 aa, Artificial Sequence
  synthetic peptide
XRWSHXWXPX

SEQ ID NO: 147 - protein, 10 aa, Artificial Sequence
  synthetic peptide
XRWSHXWXPX

SEQ ID NO: 148 - protein, 10 aa, Artificial Sequence
  synthetic peptide
XHWSHXWXPX

SEQ ID NO: 149 - protein, 10 aa, Artificial Sequence
  synthetic peptide
XHWSHXWXPX

SEQ ID NO: 150 - protein, 10 aa, Artificial Sequence
  synthetic peptide
XHWSHXWXPX

SEQ ID NO: 151 - protein, 10 aa, Artificial Sequence
  synthetic peptide
XHWSHXWYPX

SEQ ID NO: 152 - protein, 10 aa, Artificial Sequence
  synthetic peptide
XHWSHXWXPX

SEQ ID NO: 153 - protein, 10 aa, Artificial Sequence
  synthetic peptide
XHWSHXWXPX

SEQ ID NO: 154 - protein, 10 aa, Artificial Sequence
  synthetic peptide
XHWSHXWXPX

SEQ ID NO: 155 - protein, 10 aa, Artificial Sequence
  synthetic peptide
XHWSHXWXPX

SEQ ID NO: 156 - protein, 10 aa, Artificial Sequence
  synthetic peptide
XHWSHXWXPX

SEQ ID NO: 157 - protein, 10 aa, Artificial Sequence
  synthetic peptide
XHWSHXWXPX

SEQ ID NO: 158 - protein, 11 aa, Artificial Sequence
  synthetic peptide
GXHWSHRWXPX

SEQ ID NO: 159 - protein, 11 aa, Artificial Sequence
  synthetic peptide
GXHWSHRWXPX

SEQ ID NO: 160 - protein, 11 aa, Artificial Sequence
  synthetic peptide
GXHWSHRWXPX

SEQ ID NO: 161 - protein, 11 aa, Artificial Sequence
  synthetic peptide
GXHWSHRWXPX

SEQ ID NO: 162 - protein, 11 aa, Artificial Sequence
  synthetic peptide
GXHWSHRWXPX

SEQ ID NO: 163 - protein, 11 aa, Artificial Sequence
  synthetic peptide
GXHWSHRWXPX

SEQ ID NO: 164 - protein, 11 aa, Artificial Sequence
  synthetic peptide
GXHWSHRWXPX

SEQ ID NO: 165 - protein, 11 aa, Artificial Sequence
  synthetic peptide
GXHWSHRWXPX

SEQ ID NO: 166 - protein, 11 aa, Artificial Sequence
  synthetic peptide
GXHWSHRWXPX

SEQ ID NO: 167 - protein, 11 aa, Artificial Sequence
  synthetic peptide
GXHWSHRWXPX

SEQ ID NO: 168 - protein, 11 aa, Artificial Sequence
  synthetic peptide
GXHWSHRWXPX

SEQ ID NO: 169 - protein, 11 aa, Artificial Sequence
  synthetic peptide
GXHWSHRWXPX

SEQ ID NO: 170 - protein, 11 aa, Artificial Sequence
  synthetic peptide
GXHWSHRWXPX

SEQ ID NO: 171 - protein, 11 aa, Artificial Sequence
  synthetic peptide
GXHWSHRWXPX

SEQ ID NO: 172 - protein, 10 aa, Artificial Sequence
  synthetic peptide
XRWDHXWXPX

SEQ ID NO: 173 - protein, 10 aa, Artificial Sequence
  synthetic peptide
XRWXHXWXPX

SEQ ID NO: 174 - protein, 10 aa, Artificial Sequence
  synthetic peptide
XRWLHXWXPX

SEQ ID NO: 175 - protein, 10 aa, Artificial Sequence
  synthetic peptide
XRWWHXWXPX

SEQ ID NO: 176 - protein, 10 aa, Artificial Sequence
  synthetic peptide
XHWSYXWXPX

SEQ ID NO: 177 - protein, 10 aa, Artificial Sequence
  synthetic peptide
XHWSYXLXPX

SEQ ID NO: 178 - protein, 10 aa, Artificial Sequence
  synthetic peptide
XXWSXXWXPX

SEQ ID NO: 179 - protein, 9 aa, Artificial Sequence
  synthetic peptide
XWSXXWXPX

SEQ ID NO: 180 - protein, 10 aa, Artificial Sequence
  synthetic peptide
XXWSXXWXPX

SEQ ID NO: 181 - protein, 10 aa, Artificial Sequence
  synthetic peptide
XHWSXXWXPX

SEQ ID NO: 182 - protein, 10 aa, Artificial Sequence
  synthetic peptide
XRWSXXWXPX

SEQ ID NO: 183 - protein, 10 aa, Artificial Sequence
  synthetic peptide
XRWSRXWXPX

SEQ ID NO: 184 - protein, 10 aa, Artificial Sequence
  synthetic peptide
XRWSAXWXPX

SEQ ID NO: 185 - protein, 10 aa, Artificial Sequence
  synthetic peptide
XRWSLXWXPX

SEQ ID NO: 186 - protein, 10 aa, Artificial Sequence
  synthetic peptide
XRWSXXWXPX

SEQ ID NO: 187 - protein, 10 aa, Artificial Sequence
  synthetic peptide
XRLSHXWXPX

SEQ ID NO: 188 - protein, 10 aa, Artificial Sequence
  synthetic peptide
XRXSHXWXPX

SEQ ID NO: 189 - protein, 10 aa, Artificial Sequence
  synthetic peptide
XRESHXWXPX

SEQ ID NO: 190 - protein, 10 aa, Artificial Sequence
  synthetic peptide
XRXSHXWXPX

SEQ ID NO: 191 - protein, 11 aa, Artificial Sequence
  synthetic peptide
XXHWSHXWXPX

SEQ ID NO: 192 - protein, 10 aa, Artificial Sequence
  synthetic peptide
XXWSHXWXPX

SEQ ID NO: 193 - protein, 10 aa, Artificial Sequence
  synthetic peptide
XRWSHXWXPG

SEQ ID NO: 194 - protein, 10 aa, Artificial Sequence
  synthetic peptide
XRWSHXWXPP

SEQ ID NO: 195 - protein, 9 aa, Artificial Sequence
  synthetic peptide
XHWSHXWXP

SEQ ID NO: 196 - protein, 11 aa, Artificial Sequence
  synthetic peptide
GXXXSXXLXPX

SEQ ID NO: 197 - protein, 11 aa, Artificial Sequence
  synthetic peptide
GXXXSHXWYPX

SEQ ID NO: 198 - protein, 10 aa, Artificial Sequence
  synthetic peptide
XXWSHXWXPX

SEQ ID NO: 199 - protein, 10 aa, Artificial Sequence
  synthetic peptide
XHWSHXWYPX